(19) United States
(12) Patent Application Publication
(10) Pub. No.: US 2024/0392317 A1
(43) Pub. Date: Nov. 28, 2024

(54) CHIMERIC VECTORS

(71) Applicant: The United States of America, as represented by the Secretary, Department of Health and Human Servic, Bethesda, MD (US)

(72) Inventors: Shirin Munir, Potomac, MD (US); Linda G. Brock, Wyckoff, NJ (US); Ursula J. Buchholz, Silver Spring, MD (US); Peter L. Collins, Silver Spring, MD (US)

(73) Assignee: The United States of America, as represented by the Secretary, Department of Health and Human Servic, Bethesda, MD (US)

(21) Appl. No.: 18/799,179

(22) Filed: Aug. 9, 2024

Related U.S. Application Data
(63) Continuation of application No. 17/049,916, filed on Oct. 22, 2020, now Pat. No. 12,071,632.
(60) Provisional application No. 62/661,320, filed on Apr. 23, 2018.

(51) Int. Cl. C12N 15/86; A61K 39/00; A61K 39/155; A61P 31/14

(52) U.S. Cl. CPC C12N 15/86; A61K 39/155; A61P 31/14; A61K 2039/5256; A61K 2039/543; C12N 2760/18534; C12N 2760/18544; C12N 2760/18551

(57) ABSTRACT

Disclosed are live, chimeric non-human Mononegavirales vectors that allow a cell to express at least one protein from at least one human pathogen. In addition, compositions comprising the vectors, methods and kits for eliciting an immune response in a host, and methods of making the vectors are disclosed, in accordance with embodiments of the invention.

### CROSS-REFERENCE TO RELATED APPLICATIONS

This patent application is a continuation of co-pending U.S. patent application Ser. No. 17/049,916, filed Oct. 22, 2020, which is the U.S. national phase of International Patent Application No. PCT/US2019/028771, filed Apr. 23, 2019, which claims the benefit of U.S. Provisional Patent Application No. 62/661,320, filed Apr. 23, 2018, each of which are hereby incorporated by reference in their entireties.

### STATEMENT REGARDING FEDERALLY SPONSORED RESEARCH AND DEVELOPMENT

This invention was made with Government support under project number 1ZIA000372-33 by the National Institutes of Health, National Institute Allergy and Infectious Diseases. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 133,034 Byte XML file named “771449.xml,” created Jul. 15, 2024.

### BACKGROUND OF THE INVENTION

Human pathogens are a significant health concern. Despite continuous research, improved ways of preventing and treating pathogen infections are needed, especially from viruses such as human respiratory syncytial virus (“RSV”).

### BRIEF SUMMARY OF THE INVENTION

An embodiment of the invention provides live, chimeric non-human Mononegavirales vector which allows a cell to express at least one protein from at least one human pathogen.

Another embodiment of the invention provides compositions comprising the vectors of the invention and pharmaceutically acceptable carriers.

Another embodiment of the invention provides methods of eliciting an immune response to at least one human pathogen comprising administering a non-human Mononegavirales vector of the invention, or a composition of the invention, to a human.

Another embodiment of the invention provides methods of making live, chimeric non-human Mononegavirales vectors which allow a cell to express at least one protein from at least one human pathogen, comprising (a) inserting a non-native gene that encodes at least one protein from at least one human pathogen in a non-human Mononegavirales vector.

Another embodiment of the invention provides kits for eliciting an immune response, the kit comprising (a) the composition of the invention, and (b) at least one container for holding the composition.

Unexpectedly, the murine pneumonia virus (MPV) genome, as discussed herein, is an ideal vector for providing host protection against non-MPV viruses having several advantages over other types of vectors.

One advantage of the MPV genome is that it is relatively small (<15 kb) and can be easily manipulated by reverse genetics. Specifically, changes can be introduced into a cloned cDNA of the viral genome by standard recombinant DNA methods, and the resulting modified virus can be recovered in transfected tissue culture cells. In particular, one or more supernumerary genes expressing one or more heterologous antigens, such as a protective antigen from a heterologous pathogen, can be introduced into the MPV genome.

Another unexpected advantage of the MPV genome is that non-native inserts in the MPV genome are relatively stable during replication in vitro and in vivo. For example, a MPV vector with the RSV F protein inserted is very stable and despite continued investigation, deletion of the F protein sequence from the MPV genome has not yet been observed by the inventors. This is unexpected because typically the inserted sequence in some types of vector can be deleted after a few (or several) replications. Further, inserted sequences in vectors usually accumulate point mutations during replication that silence its expression; however, this occurs only sporadically in the MPV genome.

Further, several features of MPV biology make it safe to use as a vector (especially in humans). For example, MPV is a pneumotropic virus that replicates in the superficial epithelial cells of the respiratory mucosa, and thus is not a highly invasive or a systemic virus. In addition, MPV is a cytoplasmic RNA virus, and does not integrate into or otherwise perturb the host genome. Infection is acute with no known long term infection. Further, this type of virus (nonsegmented negative strand RNA virus) has very low incidence of recombination between viral genomes (Spann et al., J. Virol., 77:11201-11211 (2003) (incorporated herein in its entirety by reference)).

In addition, MPV is likely naturally attenuated in humans by host range restriction, as it is in non-human primates. Rodents are the natural hosts of MPV, and the virus is highly attenuated in non-human primates, and therefore presumably in humans, due to host-range restriction. Attenuation of a paramyxovirus or pneumovirus by host range restriction is thought to be polygenic and stable, as illustrated by the host-range restriction of bovine parainfluenza virus type 3 in non-human primates and humans (Skiadopoulos, et al., J. Virol., 77:1141-1148 (2003) (incorporated herein in its entirety by reference)).

Further, there is no evidence of human infection by MPV, and humans lack acquired immunity against this virus (Brock, et al., J. Virol., 86:5829-5843 (2012) (incorporated herein in its entirety by reference)). Despite 10-60% amino acid sequence identity between the proteins of MPV and RSV (the latter being the human pathogen that is the most closely-related to MPV), RSV-specific immunity does not cross-neutralize or cross-protect against MPV (tested in a mouse model). Thus, it is expected that use of the MPV-based vector of an embodiment of the invention in humans should not be subject to restriction by existing immunity, in particular immunity against RSV.

A further advantage is that MPV replicates in the respiratory mucosa and induces local immunity in the respiratory tract as well as systemic immunity. Accordingly, an MPV vector portends to be particularly effective against respiratory pathogens.

### BRIEF DESCRIPTION OF THE SEVERAL VIEWS OF THE DRAWING(S)

FIG. 1 depicts a diagram showing rMPV antigenomes containing the RSV F gene added as a supernumerary gene in the first (F1), third (F3), or fourth (F4) gene position. The MPV genes are shown as unfilled rectangles and the RSV F gene is shown as a shaded rectangle. The MPV gene-start (“GS”) and gene-end (“GE”) transcription signals are indicated by unfilled and filled bars, respectively, flanking each gene including the supernumerary RSV F gene. The nucleotide sequence flanking the RSV F ORF is shown under each gene map, with the following features identified: RSV F ORF (represented by a shaded box), GS and GE transcription signals (shown in bold), intergenic regions (“IG”), and the “Kozak” sequence (shown with double underlining, SEQ ID NO: 63) placed upstream of the RSV F ORF to promote efficient translation.

FIG. 2 depicts the results of a dual staining plaque assay which illustrates the stability of expression of RSV F by the rMPV vectors. Briefly, Vero cells were inoculated with serial dilutions of P1 virus stocks and incubated for four days under a 0.8% methylcellulose overlay. Monolayers were fixed and probed for RSV F and MPV antigens using specific antibodies followed by the corresponding infra-red dye-conjugated secondary antibodies. RSV F and MPV antigens appear green and red, respectively, and appear yellow when merged. One image was selected which represents the results from four independent experiments. In the upper row of plates labeled “MPV antigens,” the grey scale is indicative of the color red. In the middle row of plates labeled “RSV F,” the grey scale is indicative of the color green. In the bottom row of plates labeled “Merged,” in the plates under columns “rMPV-F1,” “rMPV-F4,” and “rMPV-F3,” the grey scale is indicative of the color yellow. In the bottom row of plates labeled “Merged,” in the plates under column “rMPV-empty,” the grey scale is indicative of the color red.

FIG. 3 depicts the multi-cycle growth kinetics of the rMPV-RSV-F vectors in human A549 lung epithelial cells. Replicate cultures of A549 cells were infected with a multiplicity of infection (“MOI”) of 0.1 plaque forming units (“PFU,” a measure of the number of infectious virus particles)/cell with rMPV-F1 (dashed line with triangles), rMPV-F3 (dotted line with open circles), rMPV-F4 (solid line with Xs), or rMPV-empty (solid line with solid black circles). At 24 hour intervals, two cultures per virus per cell line were harvested by scraping and vortexing, and clarified supernatants were prepared and flash frozen. The viral titers were subsequently determined in duplicate by plaque assay. The number of days post infection is on the x-axis and the log₁₀ PFU per ml is on the y-axis. Data are shown as mean values with the standard error of the means, although in many cases the error bars are obscured by the symbols given the small margin of error. The limit of detection was 0.7 log₁₀ PFU per mL (dotted line).

FIG. 4 depicts the multi-cycle growth kinetics of the rMPV-RSV-F vectors in Vero cells. Replicate cultures of Vero cells were infected with a MOI of 0.1 PFU/cell with rMPV-F1 (dashed line with triangles), rMPV-F3 (dotted line with open circles), rMPV-F4 (solid line with Xs), or rMPV-empty (solid line with solid black circles). At 24 hour intervals, two cultures per virus per cell line were harvested by scraping and vortexing, and clarified supernatants were prepared and flash frozen. Viral titers were subsequently determined in duplicate by plaque assay. The number of days post infection is on the x-axis and the log₁₀ PFU per ml is on the y-axis. Data are shown as mean values with the standard error of the means, although in many cases the error bars are obscured by the symbols given the small margin of error. The limit of detection was 0.7 logo PFU per mL (dotted line).

FIG. 5 depicts the cytopathic effects upon infection of Vero cells with rMPV-RSV-F vectors. Vero cell monolayers were infected with the indicated rMPV-RSV-F vectors, empty vector, or wt rRSV at an MOI of 10 PFU per cell, or mock-infected. The cultures were incubated for 96 hours at 32° C., and subjected to light photomicroscopy at a 200× magnification. The images shown are representative of two independent experiments. The mock infected and rMPV-empty infected cells do not show any signs of infection while the wt RSV, rMPV-F1, rMPV-F3, and rMPV-F4 infected cells show clear signs of viral infection.

FIG. 6 depicts the results of a Western blot used to evaluate the expression of RSV F protein and rMPV proteins in infected cells. Cell lysates were prepared at 96 hours after inoculation (“hpi”) using infected cells from the experiment shown in FIG. 5. The denatured and reduced lysates were subjected to Western blot analysis. As seen in FIG. 6, rMPV-F1, rMPV-F3, and rMPV-F4 (lanes 1, 2, and 3) all contained RSV F protein (while the rMPV-empty [lane 4] and wt rMPV [lane 5] did not).

FIG. 7 depicts the level of RSV F protein expression in infected cells. RSV F protein was detected with a mouse monoclonal antibody. The quantification plots of protein bands are from the Western blot analysis shown in FIG. 6 and are representative of three independent experiments. The relative RSV F protein expression is on the y-axis and the vectors are listed on the x-axis. The standard error is shown. As seen in FIG. 7, rMPV-F1, rMPV-F3, rMPV-F4, and wt rRSV all expressed high levels of RSV F protein.

FIG. 8 depicts the level of rMPV G, N, P, F, NS1, and NS2 protein expression in infected cells. The MPV G, N, and P proteins were detected with a hyperimmune serum raised against sucrose-gradient-purified rMPV virions. The MPV F protein was detected with a rabbit polyclonal antiserum raised against a recombinant vaccinia virus expressing only the MPV F protein. The MPV NS1 and NS2 proteins were detected with individual rabbit hyperimmune sera each raised against a synthetic peptide derived from the respective protein. Tubulin was probed as a loading control and used to normalize each sample. The quantification plots of protein bands are from the same experiment shown in FIG. 5 and are representative of three independent experiments. The relative expression of each protein is on the y-axis and the vectors are listed on the x-axis. The standard error is shown. The asterisks indicate statistical significance (p values less than 0.05). As seen in FIG. 8, the infected cells expressed each tested protein.

### DETAILED DESCRIPTION OF THE INVENTION

An embodiment of the invention provides live, chimeric non-human Mononegavirales vectors which allow a cell to express at least one protein from at least one human pathogen.

### Vectors

As used herein, a “chimeric” vector is a vector comprising non-native genetic information. For example, a non-human Mononegavirales vector which comprises genetic information or material from a human pathogen (e.g., a rodent vector which comprises genetic information or material from a human pathogen or a MPV vector which comprises genetic information from RSV).

As used herein, a “non-human Mononegavirales vector” is a virus of the order Mononegavirales whose natural host is not a human.

The vectors described herein are attenuated when provided to a human. The attenuated vectors are capable of replication in vivo but have low or no virulence in the human host. The vector attenuation may be due to any of a variety of factors that may reduce the replication and/or virulence of the vector, including, but not limited to, infection of a non-natural host (host range restriction), the presence of one or more amino acid and/or nucleotide substitutions, the addition of a heterologous gene, or the removal of part or all of a vector gene. For example, a MPV vector is attenuated in human due in part to host range restriction.

### Non-Human Mononegavirales Vectors

The non-human Mononegavirales vectors of the invention can be from any member of the order Mononegavirales as long as the natural host of the virus is a non-human animal. In an embodiment, the natural host of the virus is non-human animal. Preferably, the natural host of the virus is a non-human mammal. Preferably, the natural host of the virus is in the order Rodentia. Preferably, the natural host of the virus is in the family Muridae. Preferably, the natural host of the virus is in the subfamily Murinae. Preferably, the natural host of the virus is a mouse.

A virus is a member of the order Mononegavirales if: its genome is a linear, typically nonsegmented, single-stranded, non-infectious RNA of negative polarity that is tightly encapsidated in a ribonucleocapsid both in the infected cell and the virion; possesses inverse-complementary 3′ and 5′ termini; is not covalently linked to a protein; its genome has the characteristic gene order 3′-UTR-core protein genes-envelope protein genes-RNA-dependent RNA polymerase gene-5′-UTR (3′-N-P-M-G-L-5′); it produces 5-10 distinct mRNAs from its genome via polar sequential transcription from a single promoter located at the 3′ end of the genome; mRNAs are 5′ capped and polyadenylated; it replicates by synthesizing complete positive-sense copies of the genome, called antigenomes; it forms infectious helical ribonucleocapsids as the templates for the synthesis of mRNAs, antigenomes, and genomes; it encodes an RNA-dependent RNA polymerase (RdRp, L); and/or it typically produces enveloped virions.

The non-human Mononegavirales vectors can be from the families Pneumoviridae, Bornaviridae, Filoviridae, Paramyxovirdae, Rhabdoviridae, and those which are currently unassigned to a family.

In an embodiment, the non-human Mononegavirales vectors are from the family Pneumoviridae. The non-human Mononegavirales vectors from the family Pneumoviridae can include those from the genus Metapneumovirus, for example the species Avian metapneumovirus. The species Avian metapneumovirus includes avian metapneumovirus (“AMPV”).

The non-human Mononegavirales vectors from the family Pneumoviridae can also include those from the genus Orthopneumovirus, for example the species Bovine orthopneumovirus and Murine orthopneumovirus. The species Bovine orthopneumovirus includes bovine respiratory syncytial virus (“BRSV”). The species Murine orthopneumovirus includes murine pneumonia virus (“MPV,” previously called pneumonia virus of mice, PVM). Preferably, the non-human vector of an embodiment of the invention is MPV.

The non-human Mononegavirales vectors from the family Bornaviridae can include those from the genus Bornavirus, for example, the following species: Elapid 1 bonavirus, Mammalian 1 bonavirus, Mammalian 2 bonavirus, Passeriform 1 bonavirus, Passeriform 2 bonavirus, Psittaciform 1 bornavirus, Psittaciform 2 bornavirus, and Waterbird 1 bornavirus.

The non-human Mononegavirales vectors from the family Filoviridae can include the species Lloviu cuevavirus, from the genus Cuevairus.

The non-human Mononegavirales vectors from the family Paramyxoviridae can include those from the genus Avulavirus, for example Avian Avulavirus 1-Avian Avulavirus 13; those from the genus Henipavirus, for example the species Cedar henipavirus, Ghanaian bat henipavirus, Hendra henipavirus, Mojiang henipavirus, and Nipah heniparus (e.g., Nipah virus); those from the genus Morbillivirus; those from the genus Rubulavirus, for example the species Achimoto rubulavirus 1, Achimoto rubulavirus 2, Bat mumps rubulavirus, Canine rubulavirus, Mapuera rubulavirus, Menangly rubulavirus, Porcine rubulavirus, Simian rubulavirus, Sosuga rubulavirus, Teviot rubulavirus, Tioman rubulavirus, Tuhoko rubulavirus 1, Tuhoko rubulavirus 2, Tuhoko rubulavirus 3, and parainfluenza 5.

The non-human Mononegavirales vectors from the family Rhabdoviridae can include those from the genus Ephemerovirus, for example Bovine fever ephemerovirus; those from the genus Hapavirus; those from the genus Ledantevirus; and those from the genus Lyssavirus, for example, European bat 1 lyssavirus, European bat 2 lyssavirus, Lagos bat lyssavirus, Rabies lyssavirus, Shimoni bat lyssavirus, and West Caucasian bat lyssavirus.

In addition, the vectors of the invention can be from viruses of the order Mononegavirales that are currently unknown to one of skill in the art (i.e., those that are naturally occurring but have yet to be discovered, or those that are yet to be created either through natural or artificial processes).

In an embodiment, the non-human Mononegavirales vector is the paramyxovirus Sendai virus, and the human pathogen is not Ebola virus or respiratory syncytial virus. In an embodiment, the human pathogen is Ebola virus or respiratory syncytial virus and the non-human Mononegavirales vector is not paramyxovirus Sendai virus.

In an embodiment, the non-human Mononegavirales vector is paramyxovirus parainfluenza virus 5, and the human pathogen is not RSV, influenza, or rabies. In an embodiment, the human pathogen is RSV, influenza, or rabies, and the non-human Mononegavirales vector is not paramyxovirus parainfluenza virus 5.

In an embodiment, the non-human Mononegavirales vector is rhabdovirus vesicular stomatitis virus (“VSV”), and the human pathogen is not Ebola virus or Marburg virus. In an embodiment, the human pathogen is Ebola virus or Marburg virus, and the non-human Mononegavirales vector is not paramyxovirus rhabdovirus vesicular stomatitis virus.

In an embodiment, the non-human Mononegavirales vector is Newcastle disease virus, and the human pathogen is not influenza virus. In an embodiment, the human pathogen is influenza virus, and the non-human Mononegavirales vector is not Newcastle disease virus.

In an embodiment, the non-human Mononegavirales vector is from the family Pneumoviridae. Preferably, the non-human Mononegavirales vector is from the genus Murine orthopneumovirus. Preferably, the non-human Mononegavirales vector is MPV.

Preferably, the non-human Mononegavirales vector is a vector that infects the respiratory tract. Preferably, the non-human Mononegavirales vector is from the family Pneumoviridae and infects the respiratory tract.

In an embodiment, the non-human Mononegavirales vector is derived from wild type MPV (GenBank accession #AY729016). In an embodiment, mutations can be introduced in the MPV encoding sequence of the reverse genetic system to generate unique restriction enzyme sites for cloning (see Krempl, et al., J. Virol., 81 (17): 9490-501 (2007) (incorporated herein in its entirety by reference)). In an embodiment, the restriction enzyme sites can include AgeI and BstBI at the genome nucleotide positions 4509 and 8461, respectively. In an embodiment, the L polymerase ORF can be partly modified by codon pair optimization with a goal to increase the expression of L protein.

In an embodiment, the MPV vector has a genome promoter with at least 85% (i.e., 86%, 87%, 88%, 89%, 90%, 91%, 92%, 93%, 94%, 95%, 96%, 97%, 98%, 99%, or 100%) identity to ACGCGAAAAAATGCATAACAAAACTATCAACCTGAAAAAAGTT (SEQ ID NO: 1).

In an embodiment, the MPV vector has an antigenome promoter with at least 85% (i.e., 86%, 87%, 88%, 89%, 90%, 91%, 92%, 93%, 94%, 95%, 96%, 97%, 98%, 99%, or 100%) identity to

(SEQ ID NO: 2)
TTGATATCTCACAGGTTGTAAACATAGTTCTTTTATAATTATTGTTAGTTA
AACTATTGTGTTTGACTTCCTTTGGGTATTTTTTTCCCGT.

In an embodiment, the intergenic region between the SH and G genes and that between the M2 and L genes can be modified by introducing unique restriction enzyme sites (e.g., AgeI and BstBI), as indicated above, to create the reverse genetic system and the modified sequences are shown for these regions as shown in Table 1 below.

TABLE 1
MPV transcription signals

| Gene | Gene start | Gene end | Intergenic |
|---|---|---|---|
| NS1 | AGGACAAGTG | TAGTTAATTAAAA | CAAAGGGT |
| | (SEQ ID | (SEQ ID NO: 4) | (SEQ ID NO: 5) |
| | NO: 3) | | |
| NS2 | AGGACAAGTC | TAGTTATAGAAAAA | CATT |
| | (SEQ ID | (SEQ ID NO: 7) | (SEQ ID NO: 8) |
| | NO: 6) | | |
| N | AGGATAAATA | TATTTAATTAAAA | CTGGAAAATGT |
| | (SEQ ID | (SEQ ID NO: 10) | (SEQ ID NO: 11) |
| | NO: 9) | | |
| P | AGGATAAATA | TAGTTAATTAAAA | TAACAAC |
| | (SEQ ID | (SEQ ID NO: 13) | (SEQ ID NO: 14) |
| | NO: 12) | | |
| M | AGGACAAATA | TAGTTAAATAAAA | TC |
| | (SEQ ID | (SEQ ID NO: 16) | (SEQ ID NO: 17) |
| | NO: 15) | | |
| SH | AGGATAAATA | TAGTTAACAAAAAA | CCGGT |
| | (SEQ ID | (SEQ ID NO: 19) | (SEQ ID NO: 20) |
| | NO: 18) | | |
| G | AGGATAAGTA | TAGTTAATGAAAA | CTAAGCTTTGATATA |
| | CTATC | (SEQ ID NO: 22) | AT |
| | (SEQ ID | | (SEQ ID NO: 23) |
| | NO: 21) | | |
| F | AGGACAAATA | TAGTTAATTAAAAA | CTT |
| | (SEQ ID | (SEQ ID NO: 25) | (SEQ ID NO: 26) |
| | NO: 24) | | |
| M2 | AGGATAAGTGA | TAGTTATATAAAAA | TATTCGAATT |
| | (SEQ ID | AA | (SEQ ID NO: 29) |
| | NO: 27) | (SEQ ID NO: 28) | |
| L | AGGATCAATA | TAGTTAACAAAAAA | N/A |
| | (SEQ ID | (SEQ ID NO: 31) | |
| | NO: 30) | | |

In an embodiment, the MPV vector has transcription signal with at least 95% (i.e., 96%, 97%, 98%, 99%, or 100%) identity to SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, and/or SEQ ID NO: 31.

In an embodiment, the MPV vector comprises nucleotide sequences which encode for (listed in 3′ to 5′ gene order): non-structural protein 1 (“NS1”), non-structural protein 2 (“NS2”), nucleoprotein (“N protein”), phosphoprotein (“P protein”), matrix protein (“M protein”), small hydrophobic protein (“SH protein”), attachment protein (G protein), fusion protein (F protein), M2-1 and M2-2 proteins, and polymerase protein (“L protein”).

In an embodiment, the MPV vector has sequence for NS1 (e.g., GenBank accession #AY729016) with at least 85% (i.e., 86%, 87%, 88%, 89%, 90%, 91%, 92%, 93%, 94%, 95%, 96%, 97%, 98%, 99%, or 100%) identity to SEQ ID NO: 32.

In an embodiment, the MPV vector allows a cell to produce protein NS1 with at least 85% (i.e., 86%, 87%, 88%, 89%, 90%, 91%, 92%, 93%, 94%, 95%, 96%, 97%, 98%, 99%, or 100%) identity to SEQ ID NO: 33.

In an embodiment, the MPV vector has sequence for NS2 (e.g., GenBank accession #AY729016) with at least 85% (i.e., 86%, 87%, 88%, 89%, 90%, 91%, 92%, 93%, 94%, 95%, 96%, 97%, 98%, 99%, or 100%) identity to SEQ ID NO: 34.

In an embodiment, the MPV vector allows a cell to produce protein NS2 with at least 85% (i.e., 86%, 87%, 88%, 89%, 90%, 91%, 92%, 93%, 94%, 95%, 96%, 97%, 98%, 99%, or 100%) identity to SEQ ID NO: 35.

In an embodiment, the MPV vector has sequence for N with at least 85% (i.e., 86%, 87%, 88%, 89%, 90%, 91%, 92%, 93%, 94%, 95%, 96%, 97%, 98%, 99%, or 100%) identity to SEQ ID NO: 36.

In an embodiment, the MPV vector allows a cell to produce protein N with at least 85% (i.e., 86%, 87%, 88%, 89%, 90%, 91%, 92%, 93%, 94%, 95%, 96%, 97%, 98%, 99%, or 100%) identity to SEQ ID NO: 37.

In an embodiment, the MPV vector has sequence for P with at least 85% (i.e., 86%, 87%, 88%, 89%, 90%, 91%, 92%, 93%, 94%, 95%, 96%, 97%, 98%, 99%, or 100%) identity to SEQ ID NO: 38.

In an embodiment, the MPV vector allows a cell to produce protein P with at least 85% (i.e., 86%, 87%, 88%, 89%, 90%, 91%, 92%, 93%, 94%, 95%, 96%, 97%, 98%, 99%, or 100%) identity to SEQ ID NO: 39.

In an embodiment, the MPV vector has sequence for M with at least 85% (i.e., 86%, 87%, 88%, 89%, 90%, 91%, 92%, 93%, 94%, 95%, 96%, 97%, 98%, 99%, or 100%) identity to SEQ ID NO: 40.

In an embodiment, the MPV vector allows a cell to produce protein M with at least 85% (i.e., 86%, 87%, 88%, 89%, 90%, 91%, 92%, 93%, 94%, 95%, 96%, 97%, 98%, 99%, or 100%) identity to SEQ ID NO: 41.

In an embodiment, the MPV vector has sequence for SH with at least 85% (i.e., 86%, 87%, 88%, 89%, 90%, 91%, 92%, 93%, 94%, 95%, 96%, 97%, 98%, 99%, or 100%) identity to SEQ ID NO: 42.

In an embodiment, the MPV vector allows a cell to produce protein SH with at least 85% (i.e., 86%, 87%, 88%, 89%, 90%, 91%, 92%, 93%, 94%, 95%, 96%, 97%, 98%, 99%, or 100%) identity to SEQ ID NO: 43.

In an embodiment, the MPV vector has sequence for G with at least 85% (i.e., 86%, 87%, 88%, 89%, 90%, 91%, 92%, 93%, 94%, 95%, 96%, 97%, 98%, 99%, or 100%) identity to SEQ ID NO: 44.

In an embodiment, the MPV vector allows a cell to produce protein G with at least 85% (i.e., 86%, 87%, 88%, 89%, 90%, 91%, 92%, 93%, 94%, 95%, 96%, 97%, 98%, 99%, or 100%) identity to SEQ ID NO: 45.

In an embodiment, the MPV vector has sequence for F with at least 85% (i.e., 86%, 87%, 88%, 89%, 90%, 91%, 92%, 93%, 94%, 95%, 96%, 97%, 98%, 99%, or 100%) identity to SEQ ID NO: 46.

In an embodiment, the MPV vector allows a cell to produce F protein with at least 85% (i.e., 86%, 87%, 88%, 89%, 90%, 91%, 92%, 93%, 94%, 95%, 96%, 97%, 98%, 99%, or 100%) identity to SEQ ID NO: 47.

In an embodiment, the MPV vector has sequence for M2-1 with at least 85% (i.e., 86%, 87%, 88%, 89%, 90%, 91%, 92%, 93%, 94%, 95%, 96%, 97%, 98%, 99%, or 100%) identity to SEQ ID NO: 48.

In an embodiment, the MPV vector allows a cell to produce protein M2-1 with at least 85% (i.e., 86%, 87%, 88%, 89%, 90%, 91%, 92%, 93%, 94%, 95%, 96%, 97%, 98%, 99%, or 100%) identity to SEQ ID NO: 49.

In an embodiment, the MPV vector has sequence for M2-2 with at least 85% (i.e., 86%, 87%, 88%, 89%, 90%, 91%, 92%, 93%, 94%, 95%, 96%, 97%, 98%, 99%, or 100%) identity to SEQ ID NO: 50.

In an embodiment, the MPV vector allows a cell to produce protein M2-2 with at least 85% (i.e., 86%, 87%, 88%, 89%, 90%, 91%, 92%, 93%, 94%, 95%, 96%, 97%, 98%, 99%, or 100%) identity to SEQ ID NO: 51.

In an embodiment, the MPV vector has codon pair optimized sequence for L with at least 85% (i.e., 86%, 87%, 88%, 89%, 90%, 91%, 92%, 93%, 94%, 95%, 96%, 97%, 98%, 99%, or 100%) identity to SEQ ID NO: 52.

In an embodiment, the MPV vector allows a cell to produce protein L with at least 85% (i.e., 86%, 87%, 88%, 89%, 90%, 91%, 92%, 93%, 94%, 95%, 96%, 97%, 98%, 99%, or 100%) identity to SEQ ID NO: 53.

In some embodiment, the “Kozak” sequence (GCCGCCACC (SEQ ID NO: 63)) is upstream of the AUG start codon. The “Kozak” sequence can provide efficient context for translation initiation. Other sequences are known by one skilled in the art and can be used to increase translation efficiency.

### Human Pathogens

The vectors of the present invention can express proteins from any human pathogen. As used herein, a “human pathogen” is a pathogen which can cause an infection in a human. Human pathogens can include viruses, bacteria, protozoa, prions, and fungi.

Pathogen bacteria include those from the following species: Actinomyces israelii, Bacillus anthracis, Bacteroides fragilis, Bordetella pertussis, Borrelia, Brucella, Campylobacter jejuni, Chlamydia, Chlamydophila psittaci, Clostridium, Corynebacterium diphtheriae, Ehrlichia, Enterococcus, Escherichia, Francisella tularensis, Haemophilus influenzae, Helicobacter pylori, Klebsiella pneumoniae, Legionella pneumophila, Leptospira, Listeria monocytogenes, Mycobacterium, Mycoplasma pneumoniae, Neisseria, Pseudomonas aeruginosa, Nocardia asteroides, Rickettsia rickettsii, Salmonella, Shigella, Staphylococcus, Streptococcus, Treponema pallidum, Vibrio cholerae, and Yersinia pestis

Pathogenic fungi include those from the following species: Candida, Aspergillus, Cryptococcus, Histoplasma, Pneumocystis, and Stachybotrys.

Pathogenic protozoa include those which cause the following illnesses: malaria (by Plasmodium), amoebiasis, giardiasis, toxoplasmosis, cryptosporidiosis, trichomoniasis, Chagas disease, leishmaniasis, African trypanosomiasis, amoebic dysentery, Acanthamoeba keratitis, and primary amoebic meningoencephalitis (naegleriasis).

Pathogenic prions include those which cause the following illnesses: Creutzfeldt-Jakob disease, Iatrogenic Creutzfeldt-Jakob disease, Variant Creutzfeldt-Jakob disease, Familial Creutzfeldt-Jakob disease, Sporadic Creutzfeldt-Jakob disease, Gerstmann-Sträussler-Scheinker syndrome, Fatal familial insomnia, Kuru, Familial spongiform encephalopathy, and Multiple System Atrophy.

Pathogenic viruses include those from the following families (or orders): family Paramyxoviridae which include Human Parainfluenza Virus serotypes 1, 2, and 3 (HPIV1, 2, 3) and Measles virus; family Henipaviridae, which include Hendra virus and Nipah virus; family Orthomyxoviridae which include the avian influenza A viruses; family Coronaviridae, which include Severe Acute Respiratory Syndrome (SARS), Coronavirus and Middle East Respiratory Syndrome (MERS) Coronavirus; family Filoviridae which include Ebola virus and Marburg virus; family Arenaviridae which include Lassa Virus; order Bunyavirales (previously known as family Bunyaviridae), which include Crimean-Congo Hemorrhagic Fever Virus, Rift Valley Fever Virus, and Hantavirus; Flaviviridae and Togaviridae families, which include West Nile virus, Dengue virus, Zika virus, and Chikungunya virus; and Pneumoviridae, which include human respiratory syncytial virus (RSV) and human metapneumovirus.

Preferably, the human pathogen is a virus. Preferably, the human pathogen is a Pneumoviridae virus.

Preferably, the human pathogen is RSV. RSV is an enveloped, single stranded negative sense RNA virus. RSV possesses 10 genes that encode 11 proteins. The fusion F (“F protein”) and the attachment G (“G protein”) surface glycoproteins are the two viral neutralization antigens and are the major protective antigens. RSV F is a type I transmembrane envelope glycoprotein that mediates fusion of the virion envelope with the host cell membrane during entry.

Proteins from Human Pathogens

The at least one protein from at least one human pathogen, expressed by the cells infected by the vectors of the present invention, can include any protein which would allow the host to develop an immune response to the human pathogen.

In an embodiment, the protein is the Fusion (F) and Hemagglutinin (H) or Hemagglutinin-Neuraminidase (HN) proteins of family Paramyxoviridae, as exemplified by Human Parainfluenza Virus serotypes 1, 2, and 3 (HPIV1, 2, 3) and Measles virus.

In an embodiment, the protein is the Fusion (F) and attachment (G) proteins of family Henipaviridae, as exemplified by Hendra virus and Nipah virus.

In an embodiment, the protein is the Hemagglutinin (HA) and Neuraminidase (NA) proteins of family Orthomyxoviridae, as exemplified by highly pathogenic avian influenza A viruses.

In an embodiment, the protein is the Spike(S) protein of members of family Coronaviridae, as exemplified by Severe Acute Respiratory Syndrome (SARS), Coronavirus and Middle East Respiratory Syndrome (MERS) Coronavirus.

In an embodiment, the protein is the GP protein of members of family Filoviridae, as exemplified by Ebola virus and Marburg virus.

In an embodiment, the protein is the GP1 and GP2 proteins (expressed as a precursor GPC) of family Arenaviridae, as exemplified by Lassa Virus.

In an embodiment, the protein is the Gn (GP1) and Gc (GP2) proteins of members of Order Bunyavirales (previously known as family Bunyaviridae), as exemplified by Crimean-Congo Hemorrhagic Fever Virus, Rift Valley Fever Virus, and Hantavirus.

In an embodiment, the protein is the E glycoprotein species of members of the Flaviviridae and Togaviridae Families, as exemplified by West Nile virus, Dengue virus, Zika virus, and Chikungunya virus.

Preferably, the protein is the Fusion protein (F) or Glycoprotein (G) of family Pneumoviridae, as exemplified by respiratory syncytial virus (RSV) and metapneumovirus (MPV).

### Fusion Protein (F) of Family Pneumoviridae

There are two wild types of this protein (A and B, or A1 and B2) and many natural variants thereof. Further, the open reading frame of the nucleotide sequence can be modified by codon optimization to enhance expression of the protein. In addition, the nucleotide sequence can be modified to contain two HEK amino acid assignments (66E and 101P, see SEQ ID NO: 54) identical to those present in the wild type. Also the sequences can have mutations, deletions, and insertions as long as they do not significantly impact the ability of the expressed protein to elicit the desired immune response.

In an embodiment, the non-human Mononegavirales vector has sequence for F protein (from RSV A2) with at least 85% (i.e., 86%, 87%, 88%, 89%, 90%, 91%, 92%, 93%, 94%, 95%, 96%, 97%, 98%, 99%, or 100%) identity to SEQ ID NO: 54.

In an embodiment, the non-human Mononegavirales vector allows a cell to produce F protein (from RSV A2) with at least 85% (i.e., 86%, 87%, 88%, 89%, 90%, 91%, 92%, 93%, 94%, 95%, 96%, 97%, 98%, 99%, or 100%) identity to SEQ ID NO: 55.

In an embodiment, the non-human Mononegavirales vector has sequence for F protein (from RSV A2) with at least 85% (i.e., 86%, 87%, 88%, 89%, 90%, 91%, 92%, 93%, 94%, 95%, 96%, 97%, 98%, 99%, or 100%) identity to SEQ ID NO: 56.

In an embodiment, the non-human Mononegavirales vector allows a cell to produce F protein (from RSV A2) with at least 85% (i.e., 86%, 87%, 88%, 89%, 90%, 91%, 92%, 93%, 94%, 95%, 96%, 97%, 98%, 99%, or 100%) identity to SEQ ID NO: 57.

In an embodiment, the non-human Mononegavirales vector has sequence for F protein (from RSV B1) with at least 85% (i.e., 86%, 87%, 88%, 89%, 90%, 91%, 92%, 93%, 94%, 95%, 96%, 97%, 98%, 99%, or 100%) identity to SEQ ID NO: 58.

In an embodiment, the non-human Mononegavirales vector allows a cell to produce F protein (from RSV B1) with at least 85% (i.e., 86%, 87%, 88%, 89%, 90%, 91%, 92%, 93%, 94%, 95%, 96%, 97%, 98%, 99%, or 100%) identity to SEQ ID NO: 59.

Preferably, RSV F protein is maintained in its prefusion form which allows for an increased immune response as compared to its fusion form. RSV F protein can be stabilized in the prefusion form by introducing mutations which result in disulfide bond formation or cavity filling mutations in its globular head. Stabilized RSV F protein is less likely to unfold (which is especially desirable when it comes in contact with a host cell) (see Mclellan, et al., Science, 340:1113-1117 (2013); Mclellan, et al., Science, 342:592-598 (2013); and Joyce, et al., Nature Structural and Molecular Biology, doi: 10.1038/nsmb.3267 (2016); each of which is incorporated herein in its entirety by reference).

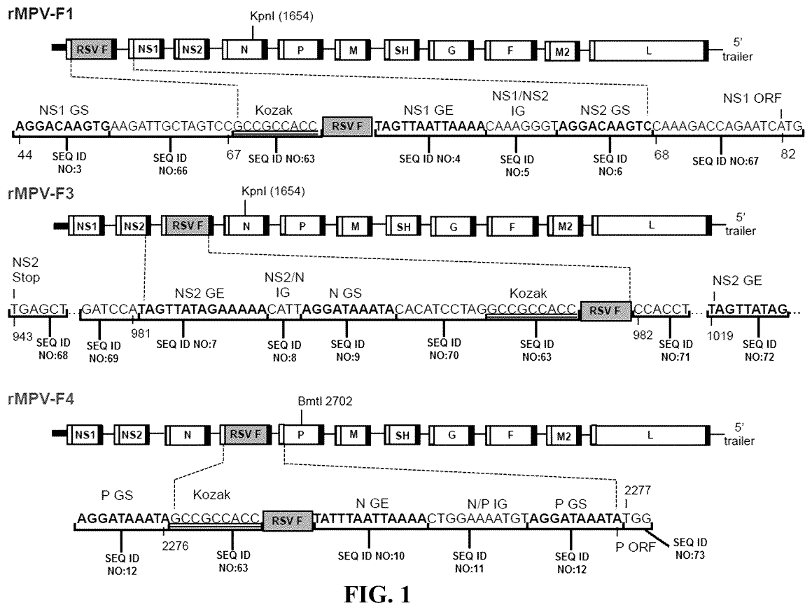

In an embodiment, the non-human Mononegavirales vector is a MPV vector and allows the cell to express F protein (of family Pneumoviridae). The sequence encoding F protein can be in any suitable place in the MPV vector genome such that F protein is expressed and provided it does not disrupt a vector gene. For example, the sequence encoding F protein can be inserted as illustrated in FIG. 1. The sequence encoding F protein should be flanked by vector gene start and gene end sequences. There can be a few or up to 200 nucleotides on either side of the open reading frame. Preferably, there about 10-20 nucleotides before the sequence encoding F protein begins to allow for ribosomes to load up thereby have efficient expression of the protein.

In an embodiment, the non-human Mononegavirales vector is a MPV vector and allows a cell to express F protein (of family Pneumoviridae) and comprises a sequence with at least 85% (i.e., 86%, 87%, 88%, 89%, 90%, 91%, 92%, 93%, 94%, 95%, 96%, 97%, 98%, 99%, or 100%) identity to SEQ ID NO: 60 (rMPV-F1).

In an embodiment, the non-human Mononegavirales vector is a MPV vector and allows a cell to express F protein (of family Pneumoviridae) and comprises a sequence with at least 85% (i.e., 86%, 87%, 88%, 89%, 90%, 91%, 92%, 93%, 94%, 95%, 96%, 97%, 98%, 99%, or 100%) identity to SEQ ID NO: 61 (rMPV-F3).

In an embodiment, the non-human Mononegavirales vector is a MPV vector and allows a cell to express F protein (of family Pneumoviridae) and comprises a sequence with at least 85% (i.e., 86%, 87%, 88%, 89%, 90%, 91%, 92%, 93%, 94%, 95%, 96%, 97%, 98%, 99%, or 100%) identity to SEQ ID NO: 62 (rMPV-F4).

In an embodiment, the cell that expresses at least one protein from at least one human pathogen is a human cell.

### Vector Compositions

An embodiment of the invention provides compositions comprising the vector of an embodiment of the invention and a pharmaceutically acceptable carrier.

The composition may be suitable for administration to a subject in need thereof. In an embodiment, the vector composition is suitable for administration to a mammal. In an embodiment, the vector is substantially free of either endotoxins or exotoxins. Endotoxins may include pyrogens, such as lipopolysaccharide (“LPS”) molecules. The vector may also be substantially free of inactive protein fragments which may cause a fever or other side effects. In embodiments, the composition contains less than about 1%, less than about 0.1%, less than about 0.01%, less than about 0.001%, or less than about 0.0001% of endotoxins, exotoxins, and/or inactive protein fragments. In some embodiments, the vector composition has lower levels of pyrogens than industrial water, tap water, or distilled water. Other vector composition components may be purified using methods known in the art, such as ion-exchange chromatography, ultrafiltration, or distillation. In other embodiments, the pyrogens may be inactivated or destroyed prior to administration to a patient. Raw materials for vector composition, such as water, buffers, salts and other chemicals may also be screened and depyrogenated. All materials in the vector composition may be sterile, and each lot of the vector composition may be tested for sterility.

In embodiments, the vector composition comprises less than about 50%, less than about 20%, less than about 10%, or less than about 5% (by dry weight) contaminating protein. In embodiments, polysaccharide capsule protein is present in the substantial absence of other biological macromolecules, such as other proteins (particularly other proteins which may substantially mask, diminish, confuse or alter the characteristics of the component proteins either as purified preparations or in their function in the subject reconstituted mixture). In some embodiments, the vector composition comprising purified subunit proteins contains less than about 5%, less than about 2%, less than about 1%, less than about 0.5%, less than about 0.2%, less than about 0.1% of protein from host cells in which the subunit proteins were expressed, relative to the amount of purified subunit.

In some embodiments, the vector has low or no toxicity to the host. In certain embodiments, the vector composition comprises ingredients at concentrations that are less than LD₅₀ measurements for the animal being vaccinated. LD₅₀ measurements may be obtained in mice or other experimental model systems, and extrapolated to humans and other animals. Methods for estimating the LD₅₀ of compounds in humans and other animals are well-known in the art. A vector composition may contain any component within it, might have an LD₅₀ value in rats of greater than about 100 g/kg, greater than about 50 g/kg, greater than about 20 g/kg, greater than about 10 g/kg, greater than about 5 g/kg, greater than about 2 g/kg, greater than about 1 g/kg, greater than about 500 mg/kg, greater than about 200 mg/kg, greater than about 100 mg/kg, greater than about 50 mg/kg, greater than about 20 mg/kg, or greater than about 10 mg/kg.

The compositions suitable for introduction of the vector vary according to route of administration. Compositions suitable for intranasal administration, such as, for example, aerosol solutions (e.g., they can be “nebulized”) and drop solutions, both of which can include sugar(s), suspending agent(s), solubilizer(s), thickening agent(s), stabilizer(s), salt buffer(s), and preservative(s).

In an embodiment, a composition comprising the vector of an embodiment of the invention comprises a monosaccharide and/or disaccharide sugar. In another embodiment, a composition comprising the vector of an embodiment of the invention comprises glucose, sucrose, and/or fructose as the sugar.

In an embodiment, a composition comprising the vector of an embodiment of the invention comprises a pharmaceutically acceptable buffer sufficient to adjust and maintain the pH of the compositions of an embodiment in the range of about 4.0 to about 8.0, preferably about 5.5 to about 7.0. Suitable buffers may include citrate, phosphate and glycine.

The vectors, alone or in combination with other suitable components, can be made into aerosol formulations to be administered via inhalation. Aerosol formulations can be placed into pressurized acceptable propellants, such as dichlorodifluoromethane, propane, nitrogen, and the like. Aerosol formulations can be delivered into the nasal passage or into the mouth.

Compositions suitable for parenteral administration, such as, for example, by intraarticular (in the joints), intravenous, intramuscular, intradermal, intraperitoneal, intranasal, and subcutaneous routes, include aqueous and non-aqueous, isotonic sterile injection solutions, which can contain antioxidants, buffers, bacteriostats, and solutes that render the formulation isotonic with the blood of the intended recipient, and general aqueous and non-aqueous sterile suspensions that can include suspending agents, solubilizers, thickening agents, stabilizers, and preservatives. The compositions can be presented in unit-dose or multi-dose sealed containers, such as ampoules and vials.

Solutions and suspensions for injection may be prepared from sterile powders, granules, and tablets. Compositions suitable for oral administration can consist of (a) liquid solutions, such as an effective amount of the polysaccharide capsule suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the vector in a flavor, usually sucrose and acacia or tragacanth, as well as pastilles comprising the vector in an inert base, such as gelatin and glycerin or sucrose and acacia emulsions, gels, and the like containing, in addition to the vectors, carriers known in the art. The vector compositions can be encapsulated, e.g., in liposomes, or in a composition that provides for slow release of the vectors.

### Vector Administration

The vectors in accordance with embodiments of the invention may be delivered by administration to an individual, typically by systemic administration (e.g., intranasal, intravenous, intraperitoneal, intramuscular, intradermal, subcutaneous, subdermal, transdermal, intracranial, mucosal, anal, vaginal, oral, buccal route or they can be inhaled) or they can be administered by topical application. In an embodiment, the route of administration is intranasal (e.g., nasal sprays or drops). In an embodiment, the route of administration is intramuscular. In other embodiments, the route of administration is subcutaneous. In yet other embodiments, the route of administration is mucosal. In certain embodiments, the route of administration is transdermal or intradermal.

### Methods for Eliciting an Immune Response

An embodiment of the invention provides methods for eliciting an immune response to at least one human pathogen comprising administering a non-human Mononegavirales vector of an embodiment of the invention, or a composition comprising the non-human Mononegavirales vector of an embodiment of the invention, to a human.

An embodiment of the invention provides methods for eliciting an immune response to at least one human pathogen comprising administering a non-human Mononegavirales vector of an embodiment of the invention, or a composition comprising the non-human Mononegavirales vector of an embodiment of the invention, to a human, wherein at the time of administration, the human does not suffer from a Pneumoviridae virus infection. In such a case, the vector of an embodiment of the invention, or a composition comprising the vector of an embodiment of the invention, is administered to a human to induce an immune response that can help protect against the establishment of a human Pneumoviridae virus, for example by protecting against infection, the first and necessary step in disease. Thus, in some aspects, the method inhibits infection by a human Pneumoviridae in an uninfected human. In another aspect, the method may reduce the duration of infection in a human who is already infected.

In certain embodiments, the vectors or compositions containing vectors of the invention confer protective immunity, allowing a vaccinated individual to exhibit delayed onset of symptoms or sequelae, or reduced severity of symptoms or sequelae, as the result of his or her exposure to the vector. In particular embodiments, individuals who have been vaccinated may display no symptoms or sequelae upon contact with a human Pneumoviridae, do not become infected by a human Pneumoviridae, or both. Protective immunity is typically achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves dendritic cells, macrophages and T lymphocytes, as well as other mechanisms involving T cells without a requirement for antibodies. In particular, cellular immunity may be mediated by T_{H1} or T_{H17} cells.

### Doses

The dose of a vector of an embodiment of the invention, or composition comprising a vector of an embodiment of the invention, is an effective amount, which induces a prophylactic response, as described above, in either a single dose or over multiple doses. Preferably, the dose is selected to minimize adverse side effects. Such an amount will vary depending upon which specific vector employed. In some embodiments, a dose comprises about 10^{8.0} infectious units (PFUs) of vector particles. In some embodiments, a dose of the composition comprises about 10^{7.0} infectious units (PFUs) of vector particles. In some embodiments, a single dose of the composition comprises about 10^{6.0} infectious units of vector particles. In some embodiments, a single dose of the composition comprises about 10^{5.0} (PFUs) infectious units of vector particles. In some embodiments, a single dose of the composition comprises about 10^{4.0} (PFUs) infectious units of vector particles. In some embodiments, a single dose of the composition comprises about 10^{3.0} (PFUs) infectious units of vector particles. In some embodiments, a single dose of the composition comprises about 10^{2.0} (PFUs) infectious units of vector particles. In some embodiments, a single dose of the composition comprises about 10, or even fewer, of infectious units (PFUs) of vector particles.

In some embodiments, a dose comprises about 10^{8.0} infectious units (50%-tissue-culture-infectious units, TCID₅₀) of vector particles. In some embodiments, a dose of the composition comprises about 10^{7.0} infectious units (TCID₅₀) of vector particles. In some embodiments, a single dose of the composition comprises about 10^{6.0} infectious units of vector particles. In some embodiments, a single dose of the composition comprises about 10^{5.0} (TCID₅₀) infectious units of vector particles. In some embodiments, a single dose of the composition comprises about 10^{4.0} (TCID₅₀) infectious units of vector particles. In some embodiments, a single dose of the composition comprises about 10^{3.0} (TCID₅₀) infectious units of vector particles. In some embodiments, a single dose of the composition comprises about 10^{2.0} (TCID₅₀) infectious units of vector particles. In some embodiments, a single dose of the composition comprises about 10, or even fewer, of infectious units (TCID₅₀) of vector particles.

The appropriate amount of vector to be delivered will depend on the age, weight, and health (e.g. immunocompromised status) of a subject. When present, typically an adjuvant will be present in amounts from 1-250 μg per dose, for example 50-150 μg, 75-125 μg or 100 μg.

In embodiments, one dose of a vector of an embodiment of the invention, or a composition comprising a vector of an embodiment of the invention, is administered to achieve the results described above. In other embodiments, following an initial vaccination, subjects receive one or more booster vaccinations, for a total of two, three, four or five vaccinations. A booster vaccination may be administered, for example, about 1 month, 2 months, 4 months, 6 months, or 12 months after the initial vaccination, such that one vaccination regimen involves administration at 0, 0.5-2, 4-8, and 12 months. It may be advantageous to administer split doses of vaccines which may be administered by the same or different routes.

In some embodiments, a vector of an embodiment of the invention, or a composition comprising a vector of any embodiment of the invention, will be administered in a dose escalation manner, such that successive administrations of the vector contain a higher concentration of vector than previous administrations. In embodiments, the vector will be administered in a manner such that successive administrations of the vector contain a lower concentration of vector than previous administrations.

In one embodiment, an effective amount is that which provides infection (as defined by the shedding of vaccine virus) in at least 20%, at least 30%, at least 40%, at least 50%, at least 60%, at least 70%, at least 80%, at least 90%, or even 100% of recipients.

In one embodiment, an effective amount is that which provides infection (as defined by a ≥4-fold increase in serum antibody titer to the vector) in at least 20%, at least 30%, at least 40%, at least 50%, at least 60%, at least 70%, at least 80%, at least 90%, or even 100% of recipients.

In one embodiment, an effective amount is that which provides infection (as defined by a ≥4-fold increase in serum antibody titer to the expressed foreign antigen) in at least 20%, at least 30%, at least 40%, at least 50%, at least 60%, at least 70%, at least 80%, at least 90%, or even 100% of recipients.

### Preparation and Storage of Vector Compositions

The vectors and compositions described herein may be produced using a variety of techniques. An embodiment of the invention provides methods for making live, chimeric non-human Mononegavirales vectors which allow a cell to express at least one protein from at least one human pathogen, comprising inserting a non-native gene that encodes at least one protein from at least one human pathogen in a non-human Mononegavirales vector. In an embodiment, the gene encoding a non-native protein from at least one human pathogen is downstream of a non-human Mononegavirales vector gene start. In an embodiment, the gene encoding a non-native protein from at least one human pathogen is upstream of a vector gene end. As used herein “downstream of a vector gene start” means that the gene encoding a protein from at least one human pathogen is inserted in a position which is downstream of a non-human Mononegavirales vector gene start such that the gene start allows the viral polymerase L protein to transcribe the gene. As used herein “upstream of a non-human Mononegavirales vector gene end” means that the gene encoding a protein from at least one human pathogen is inserted in a position which is upstream of a non-human Mononegavirales vector gene end such that the gene end allows the non-human Mononegavirales vector polymerase protein to stop transcription of the gene at the appropriate position.

In embodiments, methods for manufacturing the vector may comprise mixing one or more proteins or an immunogenic fragment or variant thereof with a carrier and/or an adjuvant.

### Kits and Components

An embodiment of the invention provides a kit for eliciting an immune response. The kit can comprise a composition of an embodiment of the invention and at least one container for holding the composition. The kit optionally, but preferably, contains instructions for using the kit to administer the composition of an embodiment of the invention to a human subject. The kit optionally contains components needed to administer the composition (e.g., syringe, needle, nebulizer, atomizer, or dropper).

Embodiments of the present subject matter described herein may be beneficial alone or in combination, with one or more other embodiments. Without limiting the foregoing description, certain non-limiting embodiments of the disclosure numbered (1)-(25) are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below:
(1) A live, chimeric non-human Mononegavirales vector which allows a cell to express at least one protein from at least one human pathogen.
(2) The non-human Mononegavirales vector of embodiment (1), wherein the natural host of the non-human Mononegavirales vector is a non-human animal.
(3) The non-human Mononegavirales vector of embodiment (1) or (2), wherein the natural host of the non-human Mononegavirales vector is from the order Rodentia.
(4) The non-human Mononegavirales vector of any one of embodiments (1)-(3), wherein the non-human Mononegavirales vector is a murine pneumonia virus (MPV).
(5) The non-human Mononegavirales vector of any one of embodiments (1)-(4), wherein the at least one human pathogen is bacteria or a virus.
(6) The non-human Mononegavirales vector of any one of embodiments (1)-(5), wherein the at least one human pathogen is a virus.
(7) The non-human Mononegavirales vector of any one of embodiments (1)-(6), wherein the at least one human pathogen is a human Pneumoviridae virus.
(8) The non-human Mononegavirales vector of any one of embodiments (1)-(7), wherein the at least one human pathogen is an Orthopneumovirus.
(9) The non-human Mononegavirales vector of any one of embodiments (1)-(8), wherein the at least one pathogen is a human respiratory syncytial virus (RSV).
(10) The non-human Mononegavirales vector of any one of embodiments (1)-(9), wherein the at least one protein is RSV F protein.
(11) The non-human Mononegavirales vector of any one of embodiments (1)-(10), wherein the non-human Mononegavirales vector comprises a sequence with at least 90% sequence identity to SEQ ID NO. 56.
(12) The non-human Mononegavirales vector of any one of embodiments (1)-(10), wherein the non-human Mononegavirales vector comprises a sequence with at least 90% sequence identity to SEQ ID NO. 58.
(13) The non-human Mononegavirales vector of any one of embodiments (1)-(9), wherein the at least one protein is RSV G protein.
(14) The non-human Mononegavirales vector of any one of embodiments (1)-(13), comprising a sequence with at least 90% sequence identity to SEQ ID NO: 60.
(15) The non-human Mononegavirales vector of any one of embodiments (1)-(13), comprising a sequence with at least 90% sequence identity to SEQ ID NO: 61.
(16) The non-human Mononegavirales vector of any one of embodiments (1)-(13), comprising a sequence with at least 90% sequence identity to SEQ ID NO: 62.
(17) A composition comprising the non-human Mononegavirales vector of any one of embodiments (1)-(16), and a pharmaceutically acceptable carrier.
(18) The composition of embodiment (17), wherein the composition is formulated for intranasal administration.
(19) A method of eliciting an immune response to at least one human pathogen comprising administering the non-human Mononegavirales vector of any one of embodiments (1)-(16), or the composition of embodiment (17) or (18), to a human.
(20) The method of embodiment (19), wherein at the time of administration, the human does not suffer from a human Pneumoviridae virus infection.
(21) The method of embodiment (19) or (20), wherein at the time of administration, the human does not suffer from a human respiratory syncytial virus (RSV) infection.
(22) The method of any one of embodiments (19)-(21), wherein the non-human Mononegavirales vector is a murine pneumonia virus (MPV).
(23) A method of making a live, chimeric non-human Mononegavirales vector which allows a cell to express at least one protein from at least one human pathogen, comprising:
(a) inserting a non-native gene that encodes at least one protein from at least one human pathogen in a non-human Mononegavirales vector.
(24) A kit for eliciting an immune response, the kit comprising:
(a) the composition of embodiment (17) or (18); and
(b) at least one container for holding the composition.
(25) A live, chimeric non-human Mononegavirales vector which allows a cell to express at least one protein from at least one human pathogen.
(26) The non-human Mononegavirales vector of embodiment (25), wherein the natural host of the non-human Mononegavirales vector is a non-human animal.
(27) The non-human Mononegavirales vector of embodiment (25), wherein the natural host of the non-human Mononegavirales vector is from the order Rodentia.
(28) The non-human Mononegavirales vector of embodiment (25), wherein the non-human Mononegavirales vector is a murine pneumonia virus (MPV).
(29) The non-human Mononegavirales vector of embodiment (25), wherein the at least one human pathogen is bacteria or a virus.
(30) The non-human Mononegavirales vector of embodiment (25), wherein the at least one human pathogen is a virus.
(31) The non-human Mononegavirales vector of embodiment (25), wherein the at least one human pathogen is a human Pneumoviridae virus.
(32) The non-human Mononegavirales vector of embodiment (25), wherein the at least one human pathogen is an Orthopneumovirus.
(33) The non-human Mononegavirales vector of embodiment (25), wherein the at least one pathogen is a human respiratory syncytial virus (RSV).
(34) The non-human Mononegavirales vector of embodiment (25), wherein the at least one protein is RSV F protein.
(35) The non-human Mononegavirales vector of embodiment (25), wherein the non-human Mononegavirales vector comprises a sequence with at least 90% sequence identity to SEQ ID NO. 56.
(36) The non-human Mononegavirales vector of embodiment (25), wherein the non-human Mononegavirales vector comprises a sequence with at least 90% sequence identity to SEQ ID NO. 58.
(37) The non-human Mononegavirales vector of embodiment (25), wherein the at least one protein is RSV G protein.
(38) The non-human Mononegavirales vector of embodiment (25), comprising a sequence with at least 90% sequence identity to SEQ ID NO: 60.
(39) The non-human Mononegavirales vector of embodiment (25), comprising a sequence with at least 90% sequence identity to SEQ ID NO: 61.
(40) The non-human Mononegavirales vector of embodiment (25), comprising a sequence with at least 90% sequence identity to SEQ ID NO: 62.
(41) A composition comprising the non-human Mononegavirales vector of embodiment (25), and a pharmaceutically acceptable carrier.
(42) The composition of embodiment (25), wherein the composition is formulated for intranasal administration.
(43) A method of eliciting an immune response to at least one human pathogen comprising administering the non-human Mononegavirales vector of embodiment (25) to a human.
(44) The method of embodiment (43), wherein at the time of administration, the human does not suffer from a human Pneumoviridae virus infection.
(45) The method of embodiment (43), wherein at the time of administration, the human does not suffer from a human respiratory syncytial virus (RSV) infection.
(46) The method of embodiment (43), wherein the non-human Mononegavirales vector is a murine pneumonia virus (MPV).
(47) A method of making a live, chimeric non-human Mononegavirales vector which allows a cell to express at least one protein from at least one human pathogen, comprising:
(a) inserting a non-native gene that encodes at least one protein from at least one human pathogen in a non-human Mononegavirales vector.
(48) A kit for eliciting an immune response, the kit comprising:
(a) the composition of embodiment (42); and
(b) at least one container for holding the composition.

The following examples further illustrate the invention but, of course, should not be construed as in any way limiting its scope.

### EXAMPLES

### Example 1

This example demonstrates how vectors of an embodiment of the invention can be prepared.

The rMPV-RSV-F vectors were constructed using a reverse genetics system as described by Kremp, et al., (“Identification of a novel virulence factor in recombinant pneumonia virus of mice,” J. Virol., (81): 9490-9501 (2007) (incorporated herein in its entirety by reference). The rMPV vector backbone used for expressing RSV F was derived from MPV (previously called PVM) strain 15 cloned in a pBluescript plasmid vector (“pBS”) that contained the two introduced restriction sites (AgeI and BstBI) that were absent in the parent strain 15 and also contained a partially modified L ORF. The downstream 67% of the L ORF was codon-pair optimized (“CPO”) to contain synonymous changes that increased the content of codon pairs associated with efficient expression in humans while preserving both the overall codon usage and the encoded amino acid sequence. The RSV F ORF from strain A2 was codon optimized for human codon usage (Genscript, Piscataway, NJ) to obtain greater protein expression. RSV F also carried the two previously described HEK amino acid assignments of 66E and 101P that make the encoded F protein amino acid sequence identical to that of an early passage of wt strain A2 and the clinical isolates.

Three MPV vector constructs were designed in which the RSV F insert was placed in the first gene position, upstream of the NS1 gene (rMPV-F1), in the third gene position, between the NS2 and N genes (rMPV-F3), or in the fourth gene position, between the N and P genes (rMPV-F4) (see FIG. 1). The RSV F inserts are positioned so that the RSV F ORF was flanked by MPV gene start (GS) and gene end (GE) transcription signals to enable transcription of RSV F as a separate mRNA. The Kozak consensus sequence GCCGCCACC (SEQ ID NO: 63) was placed upstream of the RSV F AUG start codon to provide efficient context for translation initiation as described. The RSV F inserts were synthesized commercially (Genscript) as long genome segments designed for insertion into the rMPV antigenomic plasmid using the XmaI restriction site in the plasmid (pBS) sequence upstream of the leader region and the downstream KpnI site (rMPV-F1 and rMPV-F3) or the KpnI and BmtI sites (rMPV-F4) as indicated (see FIG. 1). The final construct rMPV-F1 contains 1768 additional nucleotides, placed immediately after nucleotide position 67 (upstream of NS1), rMPV-F3 contains 1775 additional nucleotides, placed immediately after nucleotide position 981 (between NS2 and N), and rMPV-F4 contains 1771 additional nucleotides, placed immediately after nucleotide position 2276 (between N and P) of the MPV genome.

The rMPV vectors were recovered from cDNA (for an exemplary method, see Krempl, et al. supra) in BHK BSR-T7/5 cells that constitutively express the T7 RNA polymerase. The cells were transfected with the MPV antigenome plasmid and support plasmids expressing MPV N, P, M2-1, and L proteins. Twenty-four hours later, the cells were scraped, vortexed, and the cell suspension was co-cultured with a Vero cell monolayer for approximately two weeks to create P1 viral stocks. It was confirmed that the P1 virus stocks did not contain any adventitious mutations introduced during recovery. Specifically, viral RNA was isolated and Sanger sequence analysis of the complete viral genomes was performed using uncloned overlapping RT-PCR fragments. Control RT-PCR reactions lacking reverse transcriptase did not yield an amplified product, confirming that the PCR products were amplified from the viral RNA and not from cDNA used for virus rescue. Titers of virus stocks were determined by plaque assay and immunostaining.

### Example 2

This example demonstrates the in vitro stability of vectors of an embodiment of the invention.

Vero, human lung epithelial, and baby hamster kidney cells were used in his study. Vero cells (African green monkey kidney cells, CCL-81, ATCC, Manassas, VA) were maintained in OPTIPRO™ medium supplemented with 4 mM L-glutamine (ThermoFisher Scientific, Waltham, MA) and 10% fetal bovine serum (“FBS”) (Hyclone, Logan, UT). Human lung epithelial A549 (CCL-185; ATCC, Manassas, VA) cells were maintained in F-12K Medium (ATCC) supplemented with 4 mM L-glutamine. BHK BSR T7/5 cells are BHK-21 (baby hamster kidney 21) cells that were maintained in Glasgow's MEM medium (ThermoFisher Scientific) supplemented with 3% FBS. Geneticin (ThermoFisher Scientific) was included in the media for every other passage to ensure selection of T7 RNA polymerase expressing cells.

Recombinant (r) wt RSV strain A2 was used as a control. All in vitro tissue culture experiments were done at 37° C. unless otherwise noted. rMPV and the rMPV-RSV-F vectors were propagated on Vero cells by infecting at a multiplicity of infection (“MOI”) of 0.1. Virus stocks were harvested about two weeks post infection, when cytopathic effects disrupted the monolayer. rMPV titers were determined by plaque assay on Vero cells under a 0.8% methylcellulose overlay as described. Plaques were visualized by immunostaining with rabbit hyperimmune serum raised against sucrose gradient purified MPV followed by a horseradish peroxidase labeled goat anti-rabbit IgG secondary antibody (KPL, Gaithersburg, MD). Bound secondary antibodies were detected by incubation with a peroxidase substrate (KPL). Each sample was tested in duplicate and the average was reported.

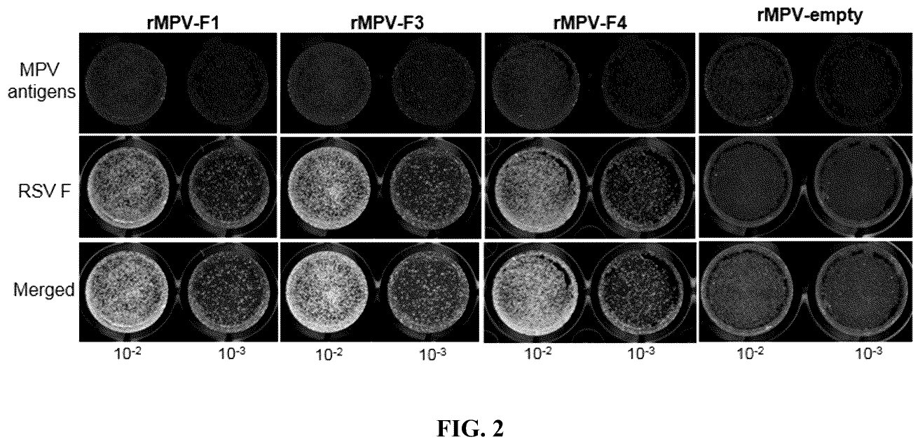

In order to evaluate the stability of expression of RSV F protein following in vitro replication, four independent viral recoveries of each rMPV-RSV-F construct were analyzed by a dual staining plaque assay to determine the stability of RSV F expression. The plaque assay was set up as previously described by Brock et al. (“Evaluation of pneumonia virus of mice as a possible human pathogen,” J. Virol., 86:5829-5843 (2012) (incorporated herein in its entirety by reference)) and cells were fixed using 80% methanol at day four post infection. To identify MPV proteins, a rabbit anti-rMPV polyclonal primary antibody described above was used at 1:5,000 and detected by a goat anti-rabbit 680 LT antibody (Li-Cor; Lincoln, NE). RSV F was probed with a mixture of three RSV F specific mouse monoclonal antibodies (1129, 1243, and 1269) each at 1:200 followed by a goat anti-mouse 800 CW secondary antibody (Li-Cor). Both secondary antibodies were used at a 1:800 dilution. The plates were scanned on an Odyssey infrared imager (Li-Cor) and the images were analyzed to determine the percentage of PFUs co-expressing RSV F and rMPV antigens. Plaque images were pseudocolored to appear red and green for MPV and RSV F antigens, respectively. On merging the two channels, rMPV plaques co-expressing RSV F would appear yellow while those with loss of RSV F expression would appear red. As seen in FIG. 2, rMPV-empty vectors did not have RSV F expression as the plaques were red. In contrast, the rMPV-F1, rMPV-F3, and rMPV-F4, were yellow when the channels were merged.

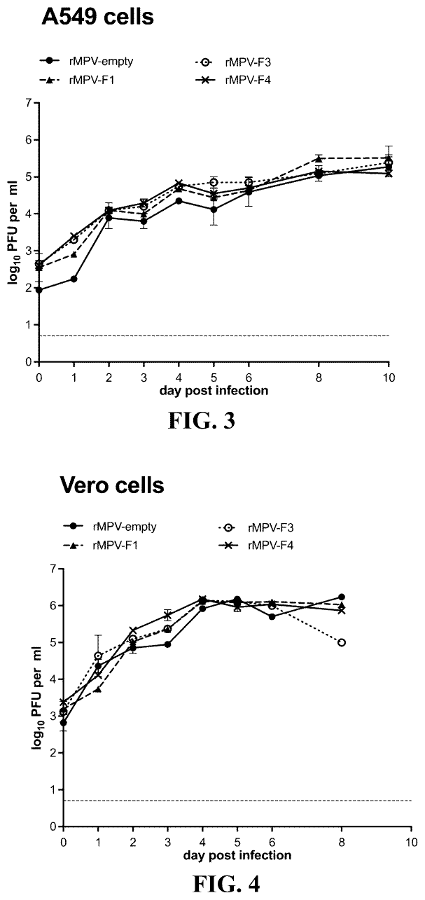

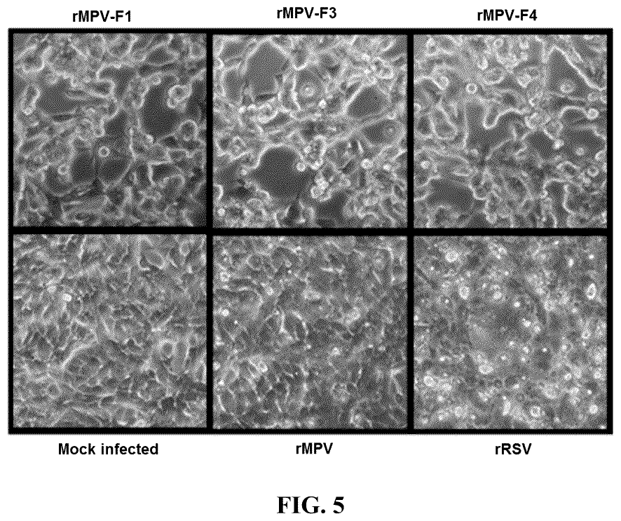

FIG. 3 shows the multi-cycle growth kinetics of the rMPV-RSV-F vectors in human A549 lung epithelial cells. FIG. 4 shows the multi-cycle growth kinetics of the rMPV-RSV-F vectors in Vero cells.

### Example 3

This example demonstrates the in vivo stability of vectors of an embodiment of the invention.

To evaluate the stability of expression of RSV F protein following in vivo replication, nasopharyngeal swab and tracheal lavage samples from the rhesus study described below were analyzed by plaque assay followed by fixation and dual stain plaque assay as described above.

In order to evaluate multi-cycle replication kinetics, replicate monolayer cultures of Vero cells and A549 cells in T25 flasks were inoculated with each virus at an MOI of 0.1 PFU/cell and incubated at 32° C. The virus inoculum was adsorbed for three hours after which monolayers were washed twice and replenished with fresh medium. On days one through six and days eight and ten for A549, and days one through 6 and day 8 for Vero cells, two cell monolayers per virus and per cell type were scraped into the supernatant, vortexed, clarified by centrifugation, and frozen. Viral titers were determined by plaque assay on Vero cells.

Next, the expression of the RSV F and MPV vector proteins was evaluated. Vero cell monolayers in 12-well plates were infected at an MOI of 10 PFU per cell with the rMPV-RSV-F vectors, empty vector, wt rMPV, wt RSV, or were mock-infected. At 96 hours post infection, images of infected cells were acquired and cell lysates were prepared to examine viral protein expression by Western blotting. Cells were washed twice with 1×PBS and lysed with 200 μL of cell lysis buffer containing 1× NUPAGE™ LDS sample buffer (ThermoFisher Scientific), 1× COMPLETE™ ULTRA protease inhibitor (Roche, Basel, Switzerland), and protease free water. Each lysate was spun through a QIAshredder spin column (Qiagen, Valencia, CA) and flash frozen on dry ice. Ninety μL of each lysate was combined with 10 μL of 10× Reducing Agent (ThermoFisher Scientific), and denatured and reduced at 70° C. for 10 min. Twenty-five μL was loaded per lane followed by SDS-PAGE and Western blot. rMPV G, N, and P proteins were detected with hyperimmune sera raised against sucrose-gradient-purified MPV virions. The MPV F protein was detected with a rabbit polyclonal antiserum raised against a recombinant vaccinia virus expressing the MPV F protein. NS1 and NS2 were detected with rabbit hyperimmune serum raised individually against the synthetic peptides TNFDRSDLET (SEQ ID NO: 64) and SDSEESGDEA (SEQ ID NO: 65) derived from the corresponding proteins, respectively. RSV F was probed with monoclonal antibodies as described by Liang et al. (“Chimeric bovine/human parainfluenza virus type 3 expressing respiratory syncytial virus (RSV) F protein: effect of insert position on expression, replication, immunogenicity, stability, and protection against RSV infection,” J. Virol. (88): 4237-4250 (2014) (incorporated herein in its entirety by reference)). Tubulin was detected as a loading control using mouse monoclonal anti-tubulin antibody (ThermoFisher Scientific, catalog number A-11126). Primary antibodies were detected with the corresponding species specific secondary antibodies conjugated with an infrared dye (Li-Cor). Membranes were scanned on an Odyssey infrared imaging system. Band signal values were obtained and background corrected using IMAGESTUDIO™ Lite, version 5.2.5 (Li-Cor).

As seen in FIG. 5, the wt RSV, rMPV-F1, rMPV-F3, and rMPV-F4 infected cells show clear signs of viral infection. The mock infected and rMPV-empty infected cells do not show any signs of infection.

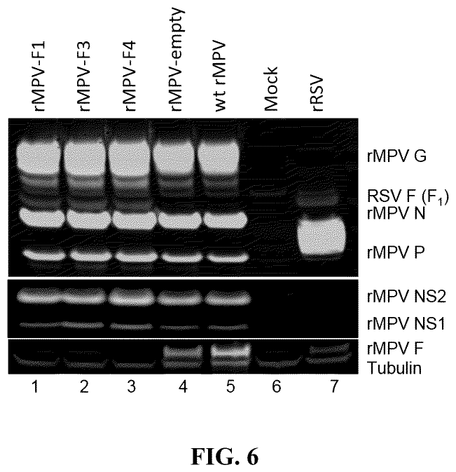

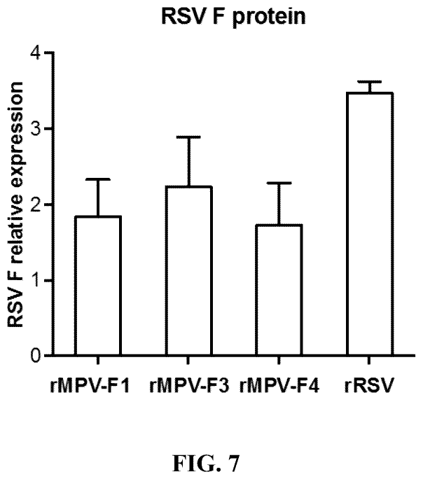

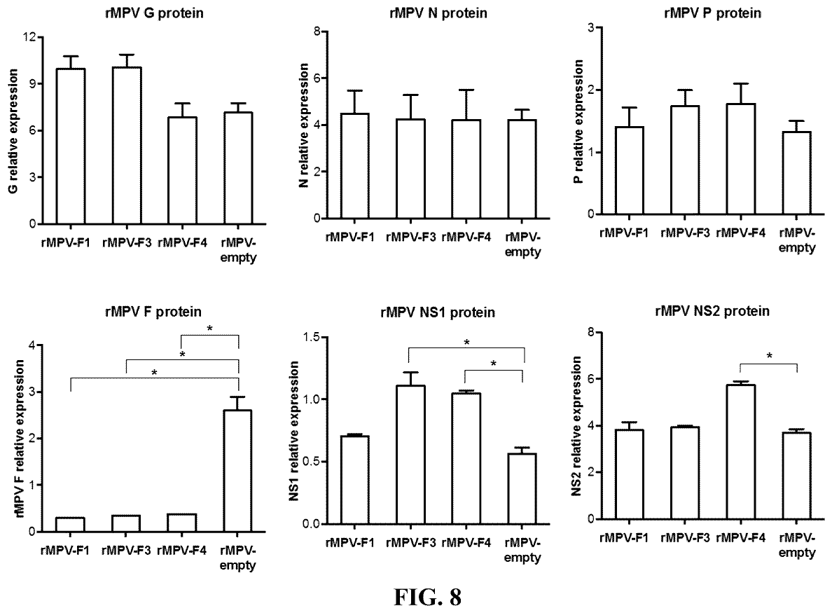

FIG. 6 shows the Western blot, specifically, it can be seen that rMPV-F1, rMPV-F3, and rMPV-F4 (lanes 1, 2, and 3) all contained RSV F protein (while the rMPV-empty [lane 4] and wt MPV [lane 5] did not). FIG. 7 depicts the level of RSV F protein expression in infected cells. As seen in FIG. 7, rMPV-F1, rMPV-F3, rMPV-F4, and wt rRSV all expressed high levels of RSV F protein. FIG. 8 depicts the level of rMPV G, N, P, F, NS1, and NS2 expression in infected cells. As seen in FIG. 8, the infected cells expressed each tested protein, some of which, were downstream of the F protein insertion. These results indicate a stable insertion.

### Example 4

This example demonstrates that vectors of an embodiment of the invention are effective at attenuating MPV in rhesus macaques.

The NIH National Institute of Allergy and Infectious Diseases Animal Care and Use Committee approved the nonhuman primate experiment described herein, which was performed in accordance with recommendations from the Guide for the Care and Use of Laboratory Animals, The National Academies Press, Washington, D.C. (2011). Eight young adult rhesus macaques (Macaca mulatta) were tested to confirm that they were seronegative for both RSV and MPV by separate PRNT₆₀ assays. Two groups of four macaques each were inoculated through the combined intranasal and intratracheal routes with 1.0 mL of inoculum per site containing 10⁶ PFUs of rMPV-F1 or rMPV-F3 diluted in L-15 medium (ThermoFisher Scientific). In a separate study, four rhesus macaques of the same cohort as those used for rMPV vectors were inoculated with 7.0 log₁₀ PFU per site of recombinant wt RSV strain A2. All four animals were pre-screened to be seronegative for RSV. Clinical observations were made daily. Nasopharyngeal (“NP”) swabs were collected on days 0-10, 12, 14, 21, and 28. Tracheal lavage (“TL”) samples were collected post-infection on days 2, 4, 6, 8, 10, 12, 14, 21, and 28. The NP and TL samples were analyzed by plaque assay as described above to quantify viral shedding. To assess the immunogenicity, sera were collected on days 0, 14, 21, and 28 days post-immunization and were analyzed for RSV- and MPV-neutralizing antibodies by PRNT₆₀.

The PRNT₆₀ (60% plaque reduction neutralization test) were performed as follows. Serum samples were analyzed for RSV- and MPV-neutralizing antibody titers by performing PRNT₆₀ assays on Vero cells using RSV-GFP and MPV-GFP, respectively. Sera from all twelve animals immunized with rMPV-F1, -F3, or wt RSV were analyzed side-by-side for RSV neutralizing antibody levels in the same experiment. Prior to use, serum samples were incubated for 30 minutes at 56° C. to inactivate the serum complement. Serial dilutions of serum were then mixed with an equal volume of diluted RSV-GFP or MPV-GFP and incubated at 37° C. for 30 min. The RSV neutralization assay was performed in the presence of 10% guinea pig complement (Lonza, Walkersville, MD). Complement was excluded from the MPV neutralization assay as it inactivates the virus. At day 6 post-infection, images of the RSV-GFP and MPV-GFP plaques were obtained by scanning on a Typhoon imager (GE Healthcare, Piscataway, NJ) and PRNT₆₀ was calculated. Each sample was tested in duplicate and the average values were reported as Log₂ PRNT₆₀.

TABLE 2
Viral titers of nasopharyngeal swab samples from the upper respiratory tract of rhesus
macaques inoculated with the indicated rMPV-RSV F vectors or with wt RSV

| | | | | | | | | | | | | Peak | |
|---|---|---|---|---|---|---|---|---|---|---|---|---|---|
| | | Virus titer (log₁₀ PFU/mL) on indicated day | | | | | | | | | | virus | Days of |
| Group | Animal ID | 1 | 2 | 3 | 4 | 5 | 6 | 7 | 8 | 9 | 10 | titer | shedding |
| rMPV-F1 | A | 2.0 | — | 2.2 | 1.6 | 2.6 | — | — | — | — | — | 2.6 | 5 |
| | B | — | — | 1.8 | 1.5 | 1.5 | 1.0 | 1.7 | — | 1.9 | — | 1.9 | 7 |
| | C | — | — | 1.7 | 1.7 | 1.5 | 1.0 | 1.6 | — | 1.2 | — | 1.7 | 7 |
| | D | — | — | — | — | — | — | — | — | — | — | — | 0 |
| | Mean ± SD: | | | | | | | | | | | 1.6 ± 1.1 | 4.8 ± 3.3 |
| rMPV-F3 | E | — | — | — | — | 1.4 | — | — | — | — | — | 1.4 | 1 |
| | F | 1.7 | 1.6 | 2.0 | 1.6 | 1.7 | 1.7 | — | 1.6 | 1.3 | — | 2.0 | 9 |
| | G | — | — | — | — | — | — | — | — | — | — | — | 0 |
| | H | 2.3 | 2.0 | 1.7 | 2.0 | 1.6 | 1.4 | — | 1.3 | 1.7 | — | 2.3 | 9 |
| | Mean ± SD: | | | | | | | | | | | 1.4 ± 1.0 | 4.8 ± 4.9 |
| wt RSV | I | 0.7 | — | 0.7 | 1.8 | 0.7 | 1.0 | 1.0 | 0.7 | — | — | 1.8 | 8 |
| | J | — | 2.4 | 3.6 | 3.3 | 1.9 | 2.2 | 2.2 | — | — | — | 3.6 | 6 |
| | K | — | 2.6 | 2.7 | 3.4 | 1.6 | 1.2 | 1.2 | — | — | — | 3.4 | 6 |
| | L | — | — | 0.7 | 1.9 | 2.0 | 1.0 | 2.2 | 1.3 | — | — | 2.2 | 6 |
| | Mean ± SD: | | | | | | | | | | | 2.8 ± 0.9 | 6.5 ± 1.0 |

Time points after day ten had no detectable virus and are not shown. The lower limit of detection was 0.7 logo PFU/mL. Samples without any detectable virus are represented as “---”. “Days of shedding” indicates the time period spanning the first day to the last day on which virus was detected, including negative days (if any) in between.

TABLE 3
Viral titers of tracheal lavage samples from the lower
respiratory tract of rhesus macaques inoculated with
the indicated rMPV-RSV F vectors or with wt RSV

| | | Virus titer | | | | | |
|---|---|---|---|---|---|---|---|
| | | (log₁₀ PFU/mL) | | | | Peak | |
| | | on indicated day | | | | virus | # of days |
| Group | Animal ID | 2 | 4 | 6 | 8 | titer | shedding |
| rMPV-F1 | A | 1.7 | — | 1.7 | — | 1.7 | 5 |
| | B | 1.0 | 1.5 | — | — | 1.5 | 3 |
| | C | 1.0 | — | 2.0 | — | 2.0 | 5 |
| | D | 2.1 | — | — | — | 2.1 | 1 |
| | Mean ± SD: | | | | | 1.8 ± 0.3 | 3.5 ± 1.9 |
| rMPV-F3 | E | 1.5 | — | 2.0 | — | 2.0 | 5 |
| | F | 2.3 | — | — | — | 2.3 | 1 |
| | G | — | — | — | — | — | 0 |
| | H | 2.6 | — | — | — | 2.6 | 1 |
| | Mean ± SD: | | | | | 1.7 ± 1.2 | 1.8 ± 2.2 |
| wt RSV | I | 3.2 | 2.5 | — | — | 3.2 | 3 |
| | J | 2.2 | 2.1 | 2.7 | — | 2.7 | 5 |
| | K | — | — | — | — | — | 0 |
| | L | 4.1 | 3.0 | 2.6 | 1.5 | 4.1 | 7 |
| | Mean ± SD: | | | | | 2.5 ± 1.8 | 4.0 ± 3.6 |

Time points after day 8 had no detectable virus and are not shown. Samples without any detectable virus are represented as “---”. The lower limit of detection was 0.7 log₁₀ PFU/mL. “#of days shedding” indicates the time period spanning the first day to the last day on which virus was detected, including negative days (if any) in between.

TABLE 4
Percentage of plaque forming units (PFU) expressing RSV F in samples inoculated collected
from the upper and lower respiratory tract of rhesus macaques with rMPV vectors
Percent PFU expressing RSV F

| | Monkey | Day | Day | Day | Day | Day | Day | Day | Day | Day |
|---|---|---|---|---|---|---|---|---|---|---|
| Virus | ID | 1 | 2 | 3 | 4 | 5 | 6 | 7 | 8 | 9 |

Nasopharyngeal swabs

| | | | | | | | | | | |
|---|---|---|---|---|---|---|---|---|---|---|
| rMPV-F1 | A | — | — | 100 | 50 | 80 | — | — | — | — |
| | B | — | — | 100 | 67 | 100 | 100 | 100 | — | 80 |
| | C | — | — | 100 | 100 | 100 | 100 | 100 | — | 50 |
| | D | — | — | — | — | — | — | — | — | — |
| rMPV-F3 | E | — | — | — | — | 100 | — | — | — | — |
| | F | 100 | 83 | 100 | 100 | 100 | 100 | — | 67 | 80 |
| | G | — | — | — | — | — | — | — | — | — |
| | H | 75 | 100 | 100 | 100 | 100 | — | — | 100 | — |

Tracheal lavage

| | | | | | | | | | | |
|---|---|---|---|---|---|---|---|---|---|---|
| rMPV-F1 | A | nc | — | nc | — | nc | 75 | nc | — | nc |
| | B | nc | — | nc | 100 | nc | — | nc | — | nc |
| | C | nc | 100 | nc | — | nc | — | nc | — | nc |
| | D | nc | 100 | nc | — | nc | — | nc | — | nc |
| rMPV-F3 | E | nc | 100 | nc | — | nc | 100 | nc | — | nc |
| | F | nc | 100 | nc | — | nc | — | nc | — | nc |
| | G | nc | — | nc | — | nc | — | nc | — | nc |
| | H | nc | 94 | nc | — | nc | — | nc | — | nc |

The nasopharyngeal swabs and tracheal lavage samples collected on the indicated days after intranasal immunization were analyzed by fluorescent dual staining plaque assay on Vero cells to determine the percentage of viral PFU co-expressing RSV F and MPV antigens during in vivo virus replication. These percent values were determined from approximately 100 plaques per sample. Samples without any detectable virus are represented as “---”. Days in which samples were not collected are designated by “nc.”

TABLE 5
RSV 60% plaque reduction neutralization titers (PRNT₆₀)
of serum samples from rhesus macaques immunized with
the rMPV-RSV-F vectors or with wt RSV
RSV PRNT (log₂ 60% titer)

| Group | ID | day 0 | day 14 | day 21 | day 28 |
|---|---|---|---|---|---|
| rMPV-F1 | A | <3.3 | 8.2 | 9.8 | 9.6 |
| | B | <3.3 | 5.4 | 6.3 | 7.3 |
| | C | <3.3 | 7.1 | 8.4 | 8.6 |
| | D | <3.3 | 6.9 | 7.9 | 7.4 |
| Mean + SD: | | <3.3 ± 0.0 | 6.9 ± 1.2 | 8.1 ± 1.4 | 8.2 ± 1.1 |
| rMPV-F3 | E | <3.3 | 5.5 | 6.2 | 7.0 |
| | F | <3.3 | 5.0 | 8.3 | 8.2 |
| | G | <3.3 | 7.6 | 8.0 | 7.8 |
| | H | <3.3 | 7.4 | 8.7 | 8.3 |
| Mean ± SD: | | <3.3 ± 0.0 | 6.4 ± 1.3 | 7.8 ± 1.1 | 7.8 ± 0.6 |
| wt RSV | I | <3.3 | 7.8 | 7.9 | 7.4 |
| | J | <3.3 | 7.8 | 8.7 | 8.3 |
| | K | <3.3 | 7.7 | 8.9 | 9.1 |
| | L | <3.3 | 8.1 | 8.7 | 8.8 |
| Mean ± SD: | | <3.3 ± 0.0 | 7.9 ± 0.2 | 8.6 ± 0.4 | 8.4 ± 0.7 |

The lower limit of detection of the RSV PRNT₆ assay was 3.3 (log₂ 60% titer). An RSV serum sample with a log₂ PRNT₆ value≥5.3 was considered positive.

TABLE 6
MPV 60% plaque reduction neutralization titers (PRNT₆₀)
of serum samples from rhesus macaques immunized with
the rMPV-RSV F vectors
MPV PRNT (log₂ 60% titer)

| Group | ID | day 0 | day 14 | day 21 | day 28 |
|---|---|---|---|---|---|
| rMPV-F1 | A | <3.3 | 13.7 | 13.8 | 13.7 |
| | B | <3.3 | 10.9 | 11.0 | 10.4 |
| | C | <3.3 | 10.7 | 11.7 | 12.5 |
| | D | <3.3 | 8.8 | 8.8 | 8.6 |
| Mean ± SD: | | <3.3 ± 0.0 | 11.0 ± 2.0 | 11.3 ± 2.1 | 11.3 ± 2.3 |
| rMPV-F3 | E | <3.3 | 11.6 | 11.2 | 11.5 |
| | F | <3.3 | 10.4 | 10.2 | 11.8 |
| | G | <3.3 | 9.5 | 9.6 | 10.5 |
| | H | <3.3 | 9.5 | 11.5 | 11.7 |
| Mean ± SD: | | <3.3 ± 0.0 | 10.3 ± 1.0 | 10.6 ± 0.9 | 11.4 ± 0.6 |

The lower limit of detection of the (PRNT₆₀) assay was 3.3 (log₂ 60% titer). An MPV serum sample with a log₂ PRNT₆₀ value≥5.3 was considered positive.

As seen in Tables 2-6, both viruses (rMPV-F1 and rMPV-F3) replicated at low levels in the upper and lower respiratory tract, maintained stable RSV F expression, and induced RSV-neutralizing serum antibodies at high levels similar to those induced by wt RSV replicating to a 5- to 25-fold higher titer. This study demonstrates that rMPV provides a highly attenuated yet immunogenic vector for the expression of RSV F protein, with potential application in RSV-naïve and RSV-experienced populations.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A live, chimeric non-human Mononegavirales vector which allows a cell to express at least one protein from at least one human pathogen.

2. The non-human Mononegavirales vector of claim 1, wherein the natural host of the non-human Mononegavirales vector is a non-human animal.

3. The non-human Mononegavirales vector of claim 1, wherein the natural host of the non-human Mononegavirales vector is from the order Rodentia.

4. The non-human Mononegavirales vector of claim 1, wherein the non-human Mononegavirales vector is a murine pneumonia virus (MPV).

5. The non-human Mononegavirales vector of claim 1, wherein the at least one human pathogen is bacteria or a virus.

6. The non-human Mononegavirales vector of claim 1, wherein the at least one human pathogen is a virus.

7. The non-human Mononegavirales vector of claim 1, wherein the at least one human pathogen is a human Pneumoviridae virus.

8. The non-human Mononegavirales vector of claim 1, wherein the at least one human pathogen is an Orthopneumovirus.

9. The non-human Mononegavirales vector of claim 1, wherein the at least one pathogen is a human respiratory syncytial virus (RSV).

10. The non-human Mononegavirales vector of claim 1, wherein the at least one protein is RSV F protein.

11. The non-human Mononegavirales vector of claim 1, wherein the at least one protein is RSV G protein.

12. The non-human Mononegavirales vector of claim 1, comprising a sequence with at least 90% sequence identity to SEQ ID NO: 60.

13. The non-human Mononegavirales vector of claim 1, comprising a sequence with at least 90% sequence identity to SEQ ID NO: 61.

14. The non-human Mononegavirales vector of claim 1, comprising a sequence with at least 90% sequence identity to SEQ ID NO: 62.

15. A composition comprising the non-human Mononegavirales vector of claim 1, and a pharmaceutically acceptable carrier.

16. The composition of claim 15, wherein the composition is formulated for intranasal administration.

17. A method of making a live, chimeric non-human Mononegavirales vector which allows a cell to express at least one protein from at least one human pathogen, comprising-inserting a non-native gene that encodes at least one protein from at least one human pathogen in a non-human Mononegavirales vector.

18. A kit for eliciting an immune response, the kit comprising:
(a) the composition of claim 15; and
(b) at least one container for holding the composition.

19. A method of eliciting an immune response to at least one human pathogen comprising administering the non-human Mononegavirales vector of claim 1 to a human.


SEQUENCE LISTING (73 sequences)

SEQ ID NO: 1 - DNA, 43 bp, Murine orthopneumovirus
  source 1..43: mol_type = genomic DNA
acgcgaaaaaatgcataacaaaactatcaacctgaaaaaagtt

SEQ ID NO: 2 - DNA, 91 bp, Murine orthopneumovirus
  source 1..91: mol_type = genomic DNA
ttgatatctcacaggttgtaaacatagttcttttataattattgttagttaaactattgtgtttgacttcctttgggtatttttttcccgt

SEQ ID NO: 3 - DNA, 10 bp, Murine orthopneumovirus
  source 1..10: mol_type = genomic DNA
aggacaagtg

SEQ ID NO: 4 - DNA, 13 bp, Murine orthopneumovirus
  source 1..13: mol_type = genomic DNA
tagttaattaaaa

SEQ ID NO: 5 - skipped

SEQ ID NO: 6 - DNA, 10 bp, Murine orthopneumovirus
  source 1..10: mol_type = genomic DNA
aggacaagtc

SEQ ID NO: 7 - DNA, 14 bp, Murine orthopneumovirus
  source 1..14: mol_type = genomic DNA
tagttatagaaaaa

SEQ ID NO: 8 - skipped

SEQ ID NO: 9 - DNA, 10 bp, Murine orthopneumovirus
  source 1..10: mol_type = genomic DNA
aggataaata

SEQ ID NO: 10 - DNA, 13 bp, Murine orthopneumovirus
  source 1..13: mol_type = genomic DNA
tatttaattaaaa

SEQ ID NO: 11 - DNA, 11 bp, Murine orthopneumovirus
  source 1..11: mol_type = genomic DNA
ctggaaaatgt

SEQ ID NO: 12 - DNA, 10 bp, Murine orthopneumovirus
  source 1..10: mol_type = genomic DNA
aggataaata

SEQ ID NO: 13 - DNA, 13 bp, Murine orthopneumovirus
  source 1..13: mol_type = genomic DNA
tagttaattaaaa

SEQ ID NO: 14 - skipped

SEQ ID NO: 15 - DNA, 10 bp, Murine orthopneumovirus
  source 1..10: mol_type = genomic DNA
aggacaaata

SEQ ID NO: 16 - DNA, 13 bp, Murine orthopneumovirus
  source 1..13: mol_type = genomic DNA
tagttaaataaaa

SEQ ID NO: 17 - skipped

SEQ ID NO: 18 - DNA, 10 bp, Murine orthopneumovirus
  source 1..10: mol_type = genomic DNA
aggataaata

SEQ ID NO: 19 - DNA, 14 bp, Murine orthopneumovirus
  source 1..14: mol_type = genomic DNA
tagttaacaaaaaa

SEQ ID NO: 20 - skipped

SEQ ID NO: 21 - DNA, 15 bp, Murine orthopneumovirus
  source 1..15: mol_type = genomic DNA
aggataagtactatc

SEQ ID NO: 22 - DNA, 13 bp, Murine orthopneumovirus
  source 1..13: mol_type = genomic DNA
tagttaatgaaaa

SEQ ID NO: 23 - DNA, 17 bp, Murine orthopneumovirus
  source 1..17: mol_type = genomic DNA
ctaagctttgatataat

SEQ ID NO: 24 - DNA, 10 bp, Murine orthopneumovirus
  source 1..10: mol_type = genomic DNA
aggacaaata

SEQ ID NO: 25 - DNA, 14 bp, Murine orthopneumovirus
  source 1..14: mol_type = genomic DNA
tagttaattaaaaa

SEQ ID NO: 26 - skipped

SEQ ID NO: 27 - DNA, 11 bp, Murine orthopneumovirus
  source 1..11: mol_type = genomic DNA
aggataagtga

SEQ ID NO: 28 - DNA, 16 bp, Murine orthopneumovirus
  source 1..16: mol_type = genomic DNA
tagttatataaaaaaa

SEQ ID NO: 29 - DNA, 10 bp, Murine orthopneumovirus
  source 1..10: mol_type = genomic DNA
tattcgaatt

SEQ ID NO: 30 - DNA, 10 bp, Murine orthopneumovirus
  source 1..10: mol_type = genomic DNA
aggatcaata

SEQ ID NO: 31 - DNA, 14 bp, Murine orthopneumovirus
  source 1..14: mol_type = genomic DNA
tagttaacaaaaaa

SEQ ID NO: 32 - DNA, 342 bp, Murine orthopneumovirus
  source 1..342: mol_type = genomic DNA
atgggctgtaatgtgatgatggagcttgattatggtggacgagctgcatggctggcattccacataaccaactttgataggtcagatttggaaactatcctaagaggtgctagggtgtgcaatacatggcaagatcagagactctccgtgtaccttgtgggaagggattgcaatttgttaagaccatttgtgcaagctgccaaatttatccataatactaggagaggccaaacattaacacattggttcacaaaaaatatcgtgtttagttctacagggcaagagacagagcctcccattgaccccacatgtgagctgttggtagagctgatcagtggttaa

SEQ ID NO: 33 - protein, 113 aa, Murine orthopneumovirus
MGCNVMMELDYGGRAAWLAFHITNFDRSDLETILRGARVCNTWQDQRLSVYLVGRDCNLLRPFVQAAKFIHNTRRGQTLTHWFTKNIVFSSTGQETEPPIDPTCELLVELISG

SEQ ID NO: 34 - DNA, 471 bp, Murine orthopneumovirus
  source 1..471: mol_type = genomic DNA
atgtccacagctatgaacaagttcactcagaccatctctaaacctgccactatcttgaatatttcagacagtgaagagtcgggcgatgaggcaggggtgggcaaggtgtcccgtaccacacagagctcagagaggtggcttgatttgctcattgaaaagttccagccgagcctacaaaacatcactagatacatcaactggaacttcatcaggatctgcaatgataggcttaaaaaagaaaaaatggggtacattgaggccaagcaatatgtggaagacatggcttggatggtgatagcatctgaggcagacagcattgagtggaagtgcataaggaggcaggagaaagtgactggggtgaaatacccaaagttcttctttgtacaacacaaagaggactggattgagtgcacaggatgcattccgtacccaggccatgacttgatctatgatgaggatgatgatgactga

SEQ ID NO: 35 - protein, 156 aa, Murine orthopneumovirus
MSTAMNKFTQTISKPATILNISDSEESGDEAGVGKVSRTTQSSERWLDLLIEKFQPSLQNITRYINWNFIRICNDRLKKEKMGYIEAKQYVEDMAWMVIASEADSIEWKCIRRQEKVTGVKYPKFFFVQHKEDWIECTGCIPYPGHDLIYDEDDDD

SEQ ID NO: 36 - DNA, 1182 bp, Murine orthopneumovirus
  source 1..1182: mol_type = genomic DNA
atgtctctagacagattgaagctcaatgatgtctcaaacaaggatagcctgctgtccaactgcaaatacagtgttaccagatccacaggcgatgtaaccagtgtgtctggtcatgctatgcagaaagcccttgcaaggacactcggcatgttcttacttactgccttcaaccgttgcgaagaagtggcagaaatagggctccaatatgccatgtccttgctaggcagagatgatagcatcaagatattaagagaagccggctacaatgtaaaatgtgtggacacacagctcaaggactttacaatcaaattacaaggaaaggaatacaaaatacaagtcctagatatagtgggaatagatgcagccaatttagctgatctagagatacaagccagaggagtggtagcaaaagaactcaaaacaggagccaggctacctgacaatcggaggcatgatgcaccagattgtggtgtgatagttctctgtattgcagcattagttgtttccaaattagctgcaggggacaggggaggacttgatgctgtggaaagaagggctttaaatgtgctgaaagccgagaaagccaggtaccccaacatggaggtcaagcagatagctgaaagtttttatgatctgtttgaaaggaagccttattacattgatgtcttcatcacttttggcctggcccagtctagtgtcaagggaggcagcaaagttgaggggctgttttcaggtctcttcatgaatgcatacggggcaggacaagttatgctgaggtggggtttactggcaaaatctgtcaagaacatcatgctaggccatgctagtgtacaagctgagatggaacaggtggttgaggtttacgaatatgctcagaagcaaggaggggaggcaggattctatcacatcagaaataatccaaaagcttcacttctctctttgaccaattgtcctaatttcaccagtgttgtgcttggcaatgctgcaggtttaggcatcatagggtcatataagggtgctcctaggaatagagaactctttgatgctgccaaagattatgcagaaagattaaaggacaacaatgtaattaactacagtgcattaaacttgactgcagaagaaagagagctgatcagccagcagctgaacattgttgatgacactcctgatgatgatatttaa

SEQ ID NO: 37 - protein, 393 aa, Murine orthopneumovirus
MSLDRLKLNDVSNKDSLLSNCKYSVTRSTGDVTSVSGHAMQKALARTLGMFLLTAFNRCEEVAEIGLQYAMSLLGRDDSIKILREAGYNVKCVDTQLKDFTIKLQGKEYKIQVLDIVGIDAANLADLEIQARGVVAKELKTGARLPDNRRHDAPDCGVIVLCIAALVVSKLAAGDRGGLDAVERRALNVLKAEKARYPNMEVKQIAESFYDLFERKPYYIDVFITFGLAQSSVKGGSKVEGLFSGLFMNAYGAGQVMLRWGLLAKSVKNIMLGHASVQAEMEQVVEVYEYAQKQGGEAGFYHIRNNPKASLLSLTNCPNFTSVVLGNAAGLGIIGSYKGAPRNRELFDAAKDYAERLKDNNVINYSALNLTAEERELISQQLNIVDDTPDDDI

SEQ ID NO: 38 - DNA, 888 bp, Murine orthopneumovirus
  source 1..888: mol_type = genomic DNA
atggagaaattcgcccccgaatttgttggcgaggatgctaacaagaaggcagaggagtttctcaaacatagatccttcccttcggaaaaaccactagctggtataccgaacactgccactcatgtcaccaaatataacatgccccctatattgcgtagctcattcaaactcccttccccgagagttgctgcaaatcttactgaaccctctgctccccctaccactccaccacccacacctccccagaacaaggaagagcagcccaaagagtctgatgttgacattgagactatgcatgtctgtaaggttcctgacaatccggaacacagcaagaagccatgctgctcagatgataccgatactaagaaaactaggaagccgatggtcacctttgtggaacccgaggagaaatttgtcggattgggagctagcttgtacagggagaccatgcagacctttgctgctgatggttatgatgaagaaagcaacctatcgtttgaggagactaaccaagagccgggttcttcatctgtagaacaaagactagatagaatagaggagaaattgtcctacataataggccttttaaacaccataatggtagcgactgctggacctaccactgctagagatgagattagagatgcccttataggcactagagaagaacttattgagatgatcaagtctgacatcttgactgtcaatgacagaatagtggccatggagaagctcagagatgaggaatgctccagagctgacactgatgatggatcagcctgttatttaacagacagagcaaggatactagataagatagtgtccagcaatgctgaagaggctaaggaagatttggatgttgatgacatcatgggcattaatttttag

SEQ ID NO: 39 - protein, 295 aa, Murine orthopneumovirus
MEKFAPEFVGEDANKKAEEFLKHRSFPSEKPLAGIPNTATHVTKYNMPPILRSSFKLPSPRVAANLTEPSAPPTTPPPTPPQNKEEQPKESDVDIETMHVCKVPDNPEHSKKPCCSDDTDTKKTRKPMVTFVEPEEKFVGLGASLYRETMQTFAADGYDEESNLSFEETNQEPGSSSVEQRLDRIEEKLSYIIGLLNTIMVATAGPTTARDEIRDALIGTREELIEMIKSDILTVNDRIVAMEKLRDEECSRADTDDGSACYLTDRARILDKIVSSNAEEAKEDLDVDDIMGINF

SEQ ID NO: 40 - DNA, 774 bp, Murine orthopneumovirus
  source 1..774: mol_type = genomic DNA
atggaggcctacttggtagagatgtaccatggtgtcccatatacagctgcagtacagctaaacttggttgaaaaacattcagccaacatatcactaactgtgtggataccgatgtttcaaacatctctaccaaagaactccgttatggacctgctacatgatgttacagtcatttgtacacagatatcaacagtgcatggtcccatgatcaaggtagatctgagctcttccaatgcaggtttagctaccatgccaaggcaattcttgataaatgctatcatagctttggatgactggggcaacatggattacgaagtgcctgttgcttttgataaaaagagcttctgtgtgacaattcttaagcctaaaaacatgctttacactgtacccagcattactcccactaatcgacctactcatgagctgatagctgtctgctctttccataacagggtaacattaaagtcattcaatatacctgtcttcatcagagcactgtctatcagacagcaggaccttgatagtgtggagcaggctataagctccgatgtggaccatgctataacaacagctagggtggctccctatgcagggcttacacttgtgatcaacatcacatccaccaaaggagcattcaaactgctaaaggcaggtagtcagattcttgcagaactgggtccctatctgacgcaggtgagcctacatgatgtgattatgaactggaaacatacaggcacttcctacatactcaagagctcctcaacaagtggatga

SEQ ID NO: 41 - protein, 257 aa, Murine orthopneumovirus
MEAYLVEMYHGVPYTAAVQLNLVEKHSANISLTVWIPMFQTSLPKNSVMDLLHDVTVICTQISTVHGPMIKVDLSSSNAGLATMPRQFLINAIIALDDWGNMDYEVPVAFDKKSFCVTILKPKNMLYTVPSITPTNRPTHELIAVCSFHNRVTLKSFNIPVFIRALSIRQQDLDSVEQAISSDVDHAITTARVAPYAGLTLVINITSTKGAFKLLKAGSQILAELGPYLTQVSLHDVIMNWKHTGTSYILKSSSTSG

SEQ ID NO: 42 - DNA, 279 bp, Murine orthopneumovirus
  source 1..279: mol_type = genomic DNA
atggatcctaacatgacctcacaccagatcaccctcgagatcaacatgaccagcagccgtattggcacatacactacaccagccccaacagctcttctccttgcatgtgccgtcatcaacacagtgtgtgcgctgataatggcctgcagcagtagaagcactgccacatcaggcattgtcagcagccaatgcacagttcatcccaatcaccctccaccaagttatggcgtcaatgtaactggtctgccgggtaacctatactcaaggaacactacataa

SEQ ID NO: 43 - protein, 92 aa, Murine orthopneumovirus
MDPNMTSHQITLEINMTSSRIGTYTTPAPTALLLACAVINTVCALIMACSSRSTATSGIVSSQCTVHPNHPPPSYGVNVTGLPGNLYSRNTT

SEQ ID NO: 44 - DNA, 1191 bp, Murine orthopneumovirus
  source 1..1191: mol_type = genomic DNA
atgggaaggaacttagaagtgagtggcagcattaccaatttgaactttgagagaactcagcatcctgacacatttaggactgttgtaaaagtgaaccaaatgtgtaagcttattgcaggtgtgctcacaagtgctgctgtggcagtttgtgtgggggtcataatgtattctgttttcacatcaaaccacaaggccaactccacgcagaatgccacgacccggaacagcacatccacccctccccaaccaaccgccggtctgcccaccacagagcaagggaccatccccagattcaccaaaccccccaccaaaaccgccacccaccatgagatcacagagcccgtcaaaatggcaacaccttcagaggatccctaccaatgctccagcaatggttatttggaccgacctgatttacctgaaaatttcaaactcgtattggatgttatatgcaagcctccaggtcctgaacatcacaacaccagctgttatgagaaacgtgaaatcaacccaggaagtgtttgccctgatcttgtaacaatgaaggcaaacatgggcttaaacaatggtggtggggaggatgctgcaccttatatagaggttaccaccctttctacgtactccaacaaaagggcaatgtgtgtccacaatgggtgtgatcaaggcttctgtttcttcctttctggtttaagcactgatcaggagagagctgtgctagagcttggaggtcaacaggctatcatggagttgcattatgattcctactggaaacactattggagtaactctaattgtgttgttcccagaacaaactgcaacctgacagaccaaactgagattttgtttcctaggtttaacaacaagaatcagtctcagtgtaccacctgtgcagattcagctggcctagataacaaattttatctcacatgtgatgggcttttaagaaccctccctctagttggactacccagcctaagtcctcaggcttacaaagctgtacccacacaaactacaggcaccaccacggcaccaacatcagagacgaggcacccaacccctgcacccaggaggtccaaacctctcagtcggaagaagagagctttatgtggtgtagactcaagcagagaacccaaaccaacaatgccttactggtgtcctatgctccaattatttccaaggaggtctaattcttaa

SEQ ID NO: 45 - protein, 396 aa, Murine orthopneumovirus
MGRNLEVSGSITNLNFERTQHPDTFRTVVKVNQMCKLIAGVLTSAAVAVCVGVIMYSVFTSNHKANSTQNATTRNSTSTPPQPTAGLPTTEQGTIPRFTKPPTKTATHHEITEPVKMATPSEDPYQCSSNGYLDRPDLPENFKLVLDVICKPPGPEHHNTSCYEKREINPGSVCPDLVTMKANMGLNNGGGEDAAPYIEVTTLSTYSNKRAMCVHNGCDQGFCFFLSGLSTDQERAVLELGGQQAIMELHYDSYWKHYWSNSNCVVPRTNCNLTDQTEILFPRFNNKNQSQCTTCADSAGLDNKFYLTCDGLLRTLPLVGLPSLSPQAYKAVPTQTTGTTTAPTSETRHPTPAPRRSKPLSRKKRALCGVDSSREPKPTMPYWCPMLQLFPRRSNS

SEQ ID NO: 46 - DNA, 1614 bp, Murine orthopneumovirus
  source 1..1614: mol_type = genomic DNA
atgattcctggcaggatctttctagtccttctggtgatcttcaacaccaagccaattcacccaaatacattaacagaaaaattctatgagtccacatgtagtgttgagactgcaggttataagagtgcccttagaacaggttggcatatgacagttatgtcaattaagttgtctcaaataaatattgagtcatgcaagagcagcaactcgttattggctcatgagcttgcaatctatagtagtgcagtggatgaattgagaacgttatcatccaatgccttgaagtccaaaaggaagaagaggttcctcggtttgattcttggtctcggagctgcagtcactgccggggtggctttagccaagacagtgcaacttgaaagtgagattgcattgattagagatgcagtgagaaatacaaatgaggctgttgttagcctaaccaacggcatgtcagtgttggctaaagtggtggatgatttgaaaaacttcatatctaaagaattactcccaaaaataaaccgagtctcttgtgatgtgcacgacatcactgccgtcattagattccaacagctcaacaaaagacttttggaagtatctcgtgaattttcatctaatgcaggattaacacacactgtttcatcttttatgttaacagaccgggaactcacctccattgtaggcggcatggctgtttcagcaggccaaaaagagataatgctatctagcaaagctataatgagaagaaatgggttagcaatattaagttcagtcaacgctgacacactggtttatgtaatacaactcccattatttggtgttatggacacagattgttgggtaataagaagttctatagactgtcataacatagcagacaagtatgcttgtttggctagagctgataatggctggtattgtcacaatgctggctcattatcatacttcccgtcgccaacggattgtgagatccacaatgggtatgctttctgtgacactctaaaaagtctaactgtacctgtaacatcacgagaatgcaactcaaacatgtataccactaactacgattgtaagatttccacaagtaaaacttatgtgagtacagcggtactgactacaatgggttgcttggtatcttgttatggtcataacagttgcacagtcatcaataatgacaaaggtataataaggactctgccagatggttgccactacatctccaacaaaggtgtggacagggttcaagtaggtaacactgtttactatcttagcaaagaagttggcaagtcaattgttgtcagaggggaaccattggtcttgaaatatgaccctttgagtttccctgacgataaatttgatgttgctataagagatgtggagcatagcatcaatcagacacgcacattcttgaaggcctctgatcagttattggacttaagtgaaaacagagagaataaaaatttaaacaagtcatatatactaacaactctgctcttcgttgtaatgcttattataataatggctgtcatagggttcattctgtataaggtattgaaaatgatcagagacaacaagttgaaatccaaaagtacacctggcctcacagttttatcatga

SEQ ID NO: 47 - protein, 537 aa, Murine orthopneumovirus
MIPGRIFLVLLVIFNTKPIHPNTLTEKFYESTCSVETAGYKSALRTGWHMTVMSIKLSQINIESCKSSNSLLAHELAIYSSAVDELRTLSSNALKSKRKKRFLGLILGLGAAVTAGVALAKTVQLESEIALIRDAVRNTNEAVVSLTNGMSVLAKVVDDLKNFISKELLPKINRVSCDVHDITAVIRFQQLNKRLLEVSREFSSNAGLTHTVSSFMLTDRELTSIVGGMAVSAGQKEIMLSSKAIMRRNGLAILSSVNADTLVYVIQLPLFGVMDTDCWVIRSSIDCHNIADKYACLARADNGWYCHNAGSLSYFPSPTDCEIHNGYAFCDTLKSLTVPVTSRECNSNMYTTNYDCKISTSKTYVSTAVLTTMGCLVSCYGHNSCTVINNDKGIIRTLPDGCHYISNKGVDRVQVGNTVYYLSKEVGKSIVVRGEPLVLKYDPLSFPDDKFDVAIRDVEHSINQTRTFLKASDQLLDLSENRENKNLNKSYILTTLLFVVMLIIIMAVIGFILYKVLKMIRDNKLKSKSTPGLTVLS

SEQ ID NO: 48 - DNA, 531 bp, Murine orthopneumovirus
  source 1..531: mol_type = genomic DNA
atgagtgtgagaccttgcaaatttgaggttcaagggttttgttccagagggaggaattgcaagtatagtcataaatattgggaatggcctttgaaaactcttatgctcaggcagaactacatgcttaataggatttataggttcctcgacaccaacacagatgcaatgtcagatgtcagcggatttgatgcaccacaaaggactgctgagtatgccttgggaaccataggtgtgctgaaaagttacctggaaaaaactaacaacatcactaaatcaatagcttgtggcagtttgatcactgtattgcagaacttggatgttggtctagtaatacaagcaagagatagcaacactgaggacaccaattacttgagaagttgcaacactatactgtcttatatagacaagatacacaagaagagacaaattattcacattctcaaaagactgccagtaggagtactatgcaatctgatccaatctgtcatctccatcgaggagaagataaattcttctatgaaaacagaatga

SEQ ID NO: 49 - protein, 176 aa, Murine orthopneumovirus
MSVRPCKFEVQGFCSRGRNCKYSHKYWEWPLKTLMLRQNYMLNRIYRFLDTNTDAMSDVSGFDAPQRTAEYALGTIGVLKSYLEKTNNITKSIACGSLITVLQNLDVGLVIQARDSNTEDTNYLRSCNTILSYIDKIHKKRQIIHILKRLPVGVLCNLIQSVISIEEKINSSMKTE

SEQ ID NO: 50 - DNA, 297 bp, Murine orthopneumovirus
  source 1..297: mol_type = genomic DNA
atgcaatctgatccaatctgtcatctccatcgaggagaagataaattcttctatgaaaacagaatgataaggctgcctaaatactatccagccatactgcataagatgtatattattagagtaaatagaaacctcacttacgatgggtctggaccatccacaataatagatgcaggaaagtctgtggtgtggaatcgtgttgatgtgatagcttgtgtgaaagaggccttgtgctgcatagaattcagctggaataaccaagtgatcatagactttgattatagccaggccagatga

SEQ ID NO: 51 - protein, 98 aa, Murine orthopneumovirus
MQSDPICHLHRGEDKFFYENRMIRLPKYYPAILHKMYIIRVNRNLTYDGSGPSTIIDAGKSVVWNRVDVIACVKEALCCIEFSWNNQVIIDFDYSQAR

SEQ ID NO: 52 - DNA, 6123 bp, Murine orthopneumovirus
  source 1..6123: mol_type = genomic DNA
atggatcctattgatgaacaagaagttaatgtgtacttgccggatagctacttaaagggtgttatatcttttagtgaaactaatgctcttggcagctgtatcattggtagacctttcttgaaggatgactttactgccactacttcaatccgtaaccccctaattgaacataaaagaataagggacactaaattagtaaaaaatattgtttcaaaccctcaatataggttagtggagcctctccaaatgcagcatgagctcttgagtgtactatcgcccaatttcatattgcacactgccaacttaaggaaaattatacaaagaagtgttgacataacagataaaaagttgaaccccattttgcacattttgaatcttaattctcctaaccaagagggtaaggtgtcggaacggctaactaggctaattaagaaacatctctctcacatacctaattgggtaagcagctggtacaatatatgggtcaatcttaacaacttactgcaggagtaccgttcaaaggaagttatagaccataactgtgttctgactaggcaattgtctggaagtttcatacatgtggtcatgagtcaatatggagttgtgataattagcaaaaaaagtaaaagatatacaatgtgtacttataaccaattcctaacctggaaggaccttgccttgagcagatttaatgccaattatgtggtctggctaagtaatgtgttaaacacactcaacgaggggttgggattaaggtgtagattaaaaggtcatctgctcagtaagttgtacatttccactgacatctttttatcttcaacatctaatgaattttataatgtggtcaaggaatttgagggcttcatcatgtcactgatattgaaacaaactgaggaagccttatttagcacaaggttttataataacatgttgaacaacttaattgatgccattgatagggctcgactagagtatctggcccgctgtgccaattcagctgccaggattaatttacctagtacagatgttatgatagcatcattgggtgatatcttatctttgataaacgttttaggtgaatccaaccttaacaacttaagtgagttatattttatcttcaggatatttggtcaccctatggttgatgagaggaaggccatggatgcagtcagagataactgttgtgaaacaaagtttctgacggctaagaaccttgcatcgttaagaggagcatatgtttatagaattatcaaaggattcgtagcaaattataacaggtggccttacataaaaactagagtttgccttacaccaacatggattaactatcttgacaccaattcatgtccctcattattagagatgacagaagatgattttattgtgttagctggagtgcactttataagagaattccacatcccaaagctaactgatctggagattatattaaatgacaaggccatatctcctccaaaatcactcatttggtcatgctttccaaaaaactacatacctcaggttatacaagatgagtatgcccggaggtattgtagagctaaagcacctttgaagacaagacgtgtcttggagttctacttacaggacaaggatttcaagttggatcagctccatagagtagtagtgaaccaggactaccttaatgataaagaacatataatttctttaacaggaaaagaaagagagttgggtgttggtaggatgtttgccatgcaacctgggaagcagaggcaagtccaaattttagcagagaagctgttggctgataacatcctgcaattctttccggagacactgactagatacggtgatttggagctgcaaaagatactagagttaaaagctggactttcaaataaaaatgacagatctaaagactcctacaataattatataagtaggtgctcattaattactgatttaagtaaatttaaccaagcttttaggtacgagtcatcctgtgtgtgtagtgatcttttagatgagctacatgggactcaaagcttattctcttggctgcatttaacagtaccactgactactataatgtgtacatataggcatgcgccgccggacactggaaacaactataatgtagatgatattgctgagcagagtggactctaccgctaccacatgggcgggattgagggctggtgccagaagctctggacaacagaggccattgctttgctagatactgtagctgtgaagggccgtttccagctaacttcattaataaatggcgacaaccaaagtattgatatttcaaaaccaacaaggctggggaccaggactcaaagtgaagcagattatgatttggcaataaattctttaagattaatatcagcagcttataaaggcattggacataaattaaaagaaggtgagacctacttgtcacgtgacatgcagttcatgagtaaaacaatacaacatgaaggggtctactacccggcctccatcaagaaaatattaagagttggtccctggatcaacacaatattagatgatataaaaacttcaacagaaagtattggttctctaactcaagaactagaatataaaggtgaaagtttaatgagcagcctgctgctgaggaacttctggctctacagattatattcagtggatttaaaagatcattctttgtgtggaaagcagctctacagatccttaataaaagtgttaaaacatttgaagaggtgcttcaacctggagaaccttggggaatgtttggaattatttttaaatgtgcccatgcagtttggaggtgctgacccaaatgtcatctacaggagcttctacagaagaactccagattttctaacagaaagtataactcatctcatcctcattttaaaacattttagaagagatttggaattcaacaaagataatgtctccaaagctgttctttctttgctagagttcaccaagaatgattctgcagaatttgtaactttgatgagagatcctcaagcaattggtagtgagaggcaggccaagatcacttcagacatcaacagaacagctgtaacttcagtgttatcaaatgctccaaatgaaatatttagaacttcagctcttcattacagcagcacagaaaatgaattaaatggaatagcaagtggagtttctcctgtttatcctcatggtcttcgagttttatatgaaagtttacctttttataaagcagagaagattgtcaacatggtttctgggaccaagtccatcaccaacatactggagaagacatcagccatctcctacacagatataattcgagccaccaacatgatggtggagaacctcactttgctaacaagaataatgaaaccaggtgctgacacatctttggatcctgacacaatagtaataacaatattatcaaaaataataagagataaatcctgggatgttggtgatataattggtgtcacttccccatctcctgtctcctgcttcaaggtggtctacacatcaactctacaaaataattcagtagtaatagaaagatacacaacagacacctacacaagaggtaagagaggccccaccaagccctgggtgggcagcagcacacaggagaagaagtccatgcctgtctacaacagacaagttttaacaagaggacaaagagatcaaatagaaaatatagcaaagctggagtgggtgttttcttcagtagcaaatattgattctttgctaaatgagctcagcaccatgactttgggtctttctctaaggaaatgtagacaactttttccaacatatttaagtttaaacttcctgcacaggctttctgtcagcagcaggcccagagaatatccttcttctcttcctgcctacaggacaacaaattttcattttgatactggaccaataaataaagtgttaacagaaagatttggagatgaagatataaatttggtatttcaaaatgcaatatcatatggtctttccaccatgtctttggtggagcagtttactggtgtctgtccaaataaagttttgctggtgcccaagctacaagaaatacaactaatgaaagttccaatatttcaaggtggcttcaacctacaaagtataattccaataataaggcagcagcacatgttcctgcccaaccacatcactccagcccagtatattgaattatttctttcttcaaaacaatttcattcaagaataaatttaaaacacaacaacagatttaaacttgttttacaaaaagattattttaatggggagaacatgatagaaactttgtccacctgtttggcaggccactggatcatcattttgatgctaatgaaggagagtcaggggatatttgacaaggagtggtatgatggttttgtaacagaccacatgttcctggacctgcagctcttcctctcctccttcaagacatttctaactgtcttcaactttgcttatttaaaagttggttcaaatatagaagaaataacaggaaatcaagccaacctgctggagctgctggacctgggctactggaagaacatgtataaagtattttcagagaccaaggtgcggctggctttgctaaaacaagatttatcatttaattctgtgaagaacagcagcagcttccggcactggtttataaattctctacaagaagtacaatgtacttctgtgccttgggtggtaaatgtaacaagaaatccaactcatttaaaaggtgttctacagtacatgaagatgatagaaagtggcatgattcaaggttattcagcaaatatttcttcagttttaagtatcccatataattatccagacatggcgcacatgatgacaaaaataataagaaatcgaggccacatgtcctatgattatccaaagatgaagaaaagtttaactttctccatgacagacatgagtgacagctacatgctcaacctcttccccaaagtagaatgttcttacatgagtggttatttggataaactagatgatactctacaacttctaaagaaacctcctgttggaagaaaagttccttctgtggctttgccctggcaccactgcaacagatacaactttgtcttcagcagcacaggctgcaaagtttctgtcattgacatgcttccaaaacatttccgaagaagtaatttaaaagtaatatgttttattggagaaggggcgggcaacctcatgctaagagctgttttggaagttggtggaaatataaaattaatatatagatccttaaaagatcctgatgatcatcatgttcctgtagaatttctaagattaaaaccttgttatccttatattgatactggtggcagtttatctttggcttcaacagatgccaccaacaaggcccactgggattatttacatcttcactggacagatcctttaaatttaatagtatgtgatgcagaaataagtggtgtgaagcactggctaaaaattcttcacaggtggtatgagcacatgacttcctgcaagcactgtttaaaatcagaacatgataaatatttaataataaaatatcatgctcaagatgatttaatagatcttcctcatggtgtcaggctactaaaatgtaacatctgcctgggctccaaactaagtggcagtgagagctacctgctcattggtcttggtctttcaaataaacttcctgtctacagtgaagttcttcattcaaaacttcttttggcagaatgtcatcagtttcatcatccaaaatatttggatgtttctggaataaatacaaatataaaatccttaataccaatgttggattatccaataacttacaacaagatcaccactttgctagaaagtgtgcgggagctttcttcaaataaaaacaagaacaccatgtggattggaagaaatcctgtctaccataataaatggctaaagaggaaatatttcaacattttaaaatggctaaaatactgtatagaacttcctgccttcaggatggattataattcatttgagagaatagaaatgctttatccaaatttaagagatttggtagattctgtttctacttcagagctgaagaaagtaataaaagtaacaggcatcctcttcaggagcaacaccatgtga

SEQ ID NO: 53 - protein, 2040 aa, Murine orthopneumovirus
MDPIDEQEVNVYLPDSYLKGVISFSETNALGSCIIGRPFLKDDFTATTSIRNPLIEHKRIRDTKLVKNIVSNPQYRLVEPLQMQHELLSVLSPNFILHTANLRKIIQRSVDITDKKLNPILHILNLNSPNQEGKVSERLTRLIKKHLSHIPNWVSSWYNIWVNLNNLLQEYRSKEVIDHNCVLTRQLSGSFIHVVMSQYGVVIISKKSKRYTMCTYNQFLTWKDLALSRFNANYVVWLSNVLNTLNEGLGLRCRLKGHLLSKLYISTDIFLSSTSNEFYNVVKEFEGFIMSLILKQTEEALFSTRFYNNMLNNLIDAIDRARLEYLARCANSAARINLPSTDVMIASLGDILSLINVLGESNLNNLSELYFIFRIFGHPMVDERKAMDAVRDNCCETKFLTAKNLASLRGAYVYRIIKGFVANYNRWPYIKTRVCLTPTWINYLDTNSCPSLLEMTEDDFIVLAGVHFIREFHIPKLTDLEIILNDKAISPPKSLIWSCFPKNYIPQVIQDEYARRYCRAKAPLKTRRVLEFYLQDKDFKLDQLHRVVVNQDYLNDKEHIISLTGKERELGVGRMFAMQPGKQRQVQILAEKLLADNILQFFPETLTRYGDLELQKILELKAGLSNKNDRSKDSYNNYISRCSLITDLSKFNQAFRYESSCVCSDLLDELHGTQSLFSWLHLTVPLTTIMCTYRHAPPDTGNNYNVDDIAEQSGLYRYHMGGIEGWCQKLWTTEAIALLDTVAVKGRFQLTSLINGDNQSIDISKPTRLGTRTQSEADYDLAINSLRLISAAYKGIGHKLKEGETYLSRDMQFMSKTIQHEGVYYPASIKKILRVGPWINTILDDIKTSTESIGSLTQELEYKGESLMSSLLLRNFWLYRLYSVDLKDHSLCGKQLYRSLIKVLKHLKRCFNLENLGECLELFLNVPMQFGGADPNVIYRSFYRRTPDFLTESITHLILILKHFRRDLEFNKDNVSKAVLSLLEFTKNDSAEFVTLMRDPQAIGSERQAKITSDINRTAVTSVLSNAPNEIFRTSALHYSSTENELNGIASGVSPVYPHGLRVLYESLPFYKAEKIVNMVSGTKSITNILEKTSAISYTDIIRATNMMVENLTLLTRIMKPGADTSLDPDTIVITILSKIIRDKSWDVGDIIGVTSPSPVSCFKVVYTSTLQNNSVVIERYTTDTYTRGKRGPTKPWVGSSTQEKKSMPVYNRQVLTRGQRDQIENIAKLEWVFSSVANIDSLLNELSTMTLGLSLRKCRQLFPTYLSLNFLHRLSVSSRPREYPSSLPAYRTTNFHFDTGPINKVLTERFGDEDINLVFQNAISYGLSTMSLVEQFTGVCPNKVLLVPKLQEIQLMKVPIFQGGFNLQSIIPIIRQQHMFLPNHITPAQYIELFLSSKQFHSRINLKHNNRFKLVLQKDYFNGENMIETLSTCLAGHWIIILMLMKESQGIFDKEWYDGFVTDHMFLDLQLFLSSFKTFLTVFNFAYLKVGSNIEEITGNQANLLELLDLGYWKNMYKVFSETKVRLALLKQDLSFNSVKNSSSFRHWFINSLQEVQCTSVPWVVNVTRNPTHLKGVLQYMKMIESGMIQGYSANISSVLSIPYNYPDMAHMMTKIIRNRGHMSYDYPKMKKSLTFSMTDMSDSYMLNLFPKVECSYMSGYLDKLDDTLQLLKKPPVGRKVPSVALPWHHCNRYNFVFSSTGCKVSVIDMLPKHFRRSNLKVICFIGEGAGNLMLRAVLEVGGNIKLIYRSLKDPDDHHVPVEFLRLKPCYPYIDTGGSLSLASTDATNKAHWDYLHLHWTDPLNLIVCDAEISGVKHWLKILHRWYEHMTSCKHCLKSEHDKYLIIKYHAQDDLIDLPHGVRLLKCNICLGSKLSGSESYLLIGLGLSNKLPVYSEVLHSKLLLAECHQFHHPKYLDVSGINTNIKSLIPMLDYPITYNKITTLLESVRELSSNKNKNTMWIGRNPVYHNKWLKRKYFNILKWLKYCIELPAFRMDYNSFERIEMLYPNLRDLVDSVSTSELKKVIKVTGILFRSNTM

SEQ ID NO: 54 - DNA, 1728 bp, Human orthopneumovirus
  source 1..1728: mol_type = genomic DNA
atggaactgctgatcctgaaagccaacgctattactactatcctgaccgccgtgacattttgcttcgcatctggacagaacattactgaggaattctaccagtcaacatgcagcgccgtgtccaaaggatacctgagcgccctgcggaccggctggtatacatcagtgattactatcgagctgtccaacatcaaggaaaacaaatgtaatgggaccgacgcaaaggtgaaactgatcaagcaggagctggataagtacaaaaatgccgtgacagaactgcagctgctgatgcagtccacaccagcaactaacaatcgcgcccggagagagctgccccggttcatgaactataccctgaacaatgctaagaaaaccaatgtgacactgtccaagaaacgcaagaggcgcttcctgggatttctgctgggcgtggggtctgccatcgctagtggagtggccgtctctaaagtcctgcacctggagggcgaagtgaacaagatcaaaagcgccctgctgtccactaacaaggcagtggtcagtctgtcaaatggcgtgtccgtcctgacctctaaggtgctggacctgaaaaattatattgataagcagctgctgcctatcgtcaacaaacagagctgctccatttctaatatcgagacagtgatcgaattccagcagaagaacaatagactgctggagattaccagagagttcagcgtgaacgccggcgtcaccacacccgtgtccacctacatgctgacaaatagtgagctgctgtcactgattaacgacatgcctatcaccaatgatcagaagaaactgatgtccaacaatgtgcagatcgtcagacagcagagttactcaatcatgtctatcattaaggaggaagtcctggcctacgtggtccagctgccactgtatggcgtgatcgacaccccctgctggaaactgcatacatctcctctgtgcactaccaacacaaaggaaggaagtaatatctgcctgactcgaaccgaccggggatggtactgtgataacgcaggcagcgtgtccttctttccacaggccgagacctgcaaggtccagagcaacagggtgttctgtgacactatgaatagcctgaccctgccttccgaagtcaacctgtgcaatgtggacatctttaatccaaagtacgattgtaagatcatgactagcaagaccgatgtcagctcctctgtgattacttctctgggggccatcgtgagttgctacggaaagacaaaatgtactgccagcaacaaaaatcgcggcatcattaagaccttctccaacgggtgcgactatgtctctaacaagggcgtggatacagtgagtgtcgggaacactctgtactatgtcaataagcaggagggaaaaagcctgtacgtgaagggcgaacccatcattaacttctatgaccccctggtgttcccttccgacgagtttgatgcatctattagtcaggtgaacgaaaaaatcaatcagagtctggcctttattcggaagtcagatgagctgctgcacaacgtgaatgctggcaaatctacaactaacatcatgatcaccacaatcatcatcgtgattatcgtcattctgctgtcactgatcgctgtggggctgctgctgtactgtaaggcaagaagcaccccagtcactctgtcaaaagaccagctgtcagggattaacaacattgccttcagtaactgatag

SEQ ID NO: 55 - protein, 574 aa, Human orthopneumovirus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 56 - DNA, 1725 bp, Human orthopneumovirus
  source 1..1725: mol_type = genomic DNA
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 57 - protein, 574 aa, Human orthopneumovirus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 58 - DNA, 1725 bp, Human orthopneumovirus
  source 1..1725: mol_type = genomic DNA
atggagctgctgatccacaggttaagtgcaatcttcctaactcttgctattaatgcattgtacctcacctcaagtcagaacataactgaggagttttaccaatcgacatgtagtgcagttagcagaggttattttagtgctttaagaacaggttggtataccagtgtcataacaatagaattaagtaatataaaagaaaccaaatgcaatggaactgacactaaagtaaaacttataaaacaagaattagataagtataagaatgcagtgacagaattacagctacttatgcaaaacacaccagctgccaacaaccgggccagaagagaagcaccacagtatatgaactatacaatcaataccactaaaaacctaaatgtatcaataagcaagaagaggaaacgaagatttctgggcttcttgttaggtgtaggatctgcaatagcaagtggtatagctgtatccaaagttctacaccttgaaggagaagtgaacaagatcaaaaatgctttgttatctacaaacaaagctgtagtcagtctatcaaatggggtcagtgttttaaccagcaaagtgttagatctcaagaattacataaataaccaattattacccatagtaaatcaacagagctgtcgcatctccaacattgaaacagttatagaattccagcagaagaacagcagattgttggaaatcaacagagaattcagtgtcaatgcaggtgtaacaacacctttaagcacttacatgttaacaaacagtgagttactatcattgatcaatgatatgcctataacaaatgatcagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttctttccacaggctgacacttgtaaagtacagtccaatcgagtattttgtgacactatgaacagtttgacattaccaagtgaagtcagcctttgtaacactgacatattcaattccaagtatgactgcaaaattatgacatcaaaaacagacataagcagctcagtaattacttctcttggagctatagtgtcatgctatggtaaaactaaatgcactgcatccaacaaaaatcgtgggattataaagacattttctaatggttgtgactatgtgtcaaacaaaggagtagatactgtgtcagtgggcaacactttatactatgtaaacaagctggaaggcaagaacctttatgtaaaaggggaacctataataaattactatgaccctctagtgtttccttctgatgagtttgatgcatcaatatctcaagtcaatgaaaaaatcaatcaaagtttagcttttattcgtagatctgatgaattactacataatgtaaatactggcaaatctactacaaatattatgataactacaattattatagtaatcattgtagtattgttatcattaatagctattggtttgctgttgtattgcaaagccaaaaacacaccagttacactaagcaaagaccaactaagtggaatcaataatattgcattcagcaaatag

SEQ ID NO: 59 - protein, 574 aa, Human orthopneumovirus
MELLIHRLSAIFLTLAINALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEINREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 60 - DNA, 16658 bp, synthetic construct
  misc_feature 1..16658: note = Chimeric Vector
  source 1..16658: mol_type = other DNA
acgcgaaaaaatgcataacaaaactatcaacctgaaaaaagttaggacaagtgaagattgctagtccgccgccaccatggaactgctgatcctgaaagccaacgctattactactatcctgaccgccgtgacattttgcttcgcatctggacagaacattactgaggaattctaccagtcaacatgcagcgccgtgtccaaaggatacctgagcgccctgcggaccggctggtatacatcagtgattactatcgagctgtccaacatcaaggaaaacaaatgtaatgggaccgacgcaaaggtgaaactgatcaagcaggagctggataagtacaaaaatgccgtgacagaactgcagctgctgatgcagtccacaccagcaactaacaatcgcgcccggagagagctgccccggttcatgaactataccctgaacaatgctaagaaaaccaatgtgacactgtccaagaaacgcaagaggcgcttcctgggatttctgctgggcgtggggtctgccatcgctagtggagtggccgtctctaaagtcctgcacctggagggcgaagtgaacaagatcaaaagcgccctgctgtccactaacaaggcagtggtcagtctgtcaaatggcgtgtccgtcctgacctctaaggtgctggacctgaaaaattatattgataagcagctgctgcctatcgtcaacaaacagagctgctccatttctaatatcgagacagtgatcgaattccagcagaagaacaatagactgctggagattaccagagagttcagcgtgaacgccggcgtcaccacacccgtgtccacctacatgctgacaaatagtgagctgctgtcactgattaacgacatgcctatcaccaatgatcagaagaaactgatgtccaacaatgtgcagatcgtcagacagcagagttactcaatcatgtctatcattaaggaggaagtcctggcctacgtggtccagctgccactgtatggcgtgatcgacaccccctgctggaaactgcatacatctcctctgtgcactaccaacacaaaggaaggaagtaatatctgcctgactcgaaccgaccggggatggtactgtgataacgcaggcagcgtgtccttctttccacaggccgagacctgcaaggtccagagcaacagggtgttctgtgacactatgaatagcctgaccctgccttccgaagtcaacctgtgcaatgtggacatctttaatccaaagtacgattgtaagatcatgactagcaagaccgatgtcagctcctctgtgattacttctctgggggccatcgtgagttgctacggaaagacaaaatgtactgccagcaacaaaaatcgcggcatcattaagaccttctccaacgggtgcgactatgtctctaacaagggcgtggatacagtgagtgtcgggaacactctgtactatgtcaataagcaggagggaaaaagcctgtacgtgaagggcgaacccatcattaacttctatgaccccctggtgttcccttccgacgagtttgatgcatctattagtcaggtgaacgaaaaaatcaatcagagtctggcctttattcggaagtcagatgagctgctgcacaacgtgaatgctggcaaatctacaactaacatcatgatcaccacaatcatcatcgtgattatcgtcattctgctgtcactgatcgctgtggggctgctgctgtactgtaaggcaagaagcaccccagtcactctgtcaaaagaccagctgtcagggattaacaacattgccttcagtaactgatagtagttaattaaaacaaagggtaggacaagtccaaagaccagaatcatgggctgtaatgtgatgatggagcttgattatggtggacgagctgcatggctggcattccacataaccaactttgataggtcagatttggaaactatcctaagaggtgctagggtgtgcaatacatggcaagatcagagactctccgtgtaccttgtgggaagggattgcaatttgttaagaccatttgtgcaagctgccaaatttatccataatactaggagaggccaaacattaacacattggttcacaaaaaatatcgtgtttagttctacagggcaagagacagagcctcccattgaccccacatgtgagctgttggtagagctgatcagtggttaagatgacagcacattatgtagttaattaaaacaaagggtaggacaagtcctaatgtccacagctatgaacaagttcactcagaccatctctaaacctgccactatcttgaatatttcagacagtgaagagtcgggcgatgaggcaggggtgggcaaggtgtcccgtaccacacagagctcagagaggtggcttgatttgctcattgaaaagttccagccgagcctacaaaacatcactagatacatcaactggaacttcatcaggatctgcaatgataggcttaaaaaagaaaaaatggggtacattgaggccaagcaatatgtggaagacatggcttggatggtgatagcatctgaggcagacagcattgagtggaagtgcataaggaggcaggagaaagtgactggggtgaaatacccaaagttcttctttgtacaacacaaagaggactggattgagtgcacaggatgcattccgtacccaggccatgacttgatctatgatgaggatgatgatgactgagctgactcagatcactccaaccagcagagagatccaccacctatagcaagtgatgtagtatttagtctatgattagttatagaaaaacattaggataaatacacatcctaggccgggccaaaatgtctctagacagattgaagctcaatgatgtctcaaacaaggatagcctgctgtccaactgcaaatacagtgttaccagatccacaggcgatgtaaccagtgtgtctggtcatgctatgcagaaagcccttgcaaggacactcggcatgttcttacttactgccttcaaccgttgcgaagaagtggcagaaatagggctccaatatgccatgtccttgctaggcagagatgatagcatcaagatattaagagaagccggctacaatgtaaaatgtgtggacacacagctcaaggactttacaatcaaattacaaggaaaggaatacaaaatacaagtcctagatatagtgggaatagatgcagccaatttagctgatctagagatacaagccagaggagtggtagcaaaagaactcaaaacaggagccaggctacctgacaatcggaggcatgatgcaccagattgtggtgtgatagttctctgtattgcagcattagttgtttccaaattagctgcaggggacaggggaggacttgatgctgtggaaagaagggctttaaatgtgctgaaagccgagaaagccaggtaccccaacatggaggtcaagcagatagctgaaagtttttatgatctgtttgaaaggaagccttattacattgatgtcttcatcacttttggcctggcccagtctagtgtcaagggaggcagcaaagttgaggggctgttttcaggtctcttcatgaatgcatacggggcaggacaagttatgctgaggtggggtttactggcaaaatctgtcaagaacatcatgctaggccatgctagtgtacaagctgagatggaacaggtggttgaggtttacgaatatgctcagaagcaaggaggggaggcaggattctatcacatcagaaataatccaaaagcttcacttctctctttgaccaattgtcctaatttcaccagtgttgtgcttggcaatgctgcaggtttaggcatcatagggtcatataagggtgctcctaggaatagagaactctttgatgctgccaaagattatgcagaaagattaaaggacaacaatgtaattaactacagtgcattaaacttgactgcagaagaaagagagctgatcagccagcagctgaacattgttgatgacactcctgatgatgatatttaattaaaactggaaaatgtaggataaatatggagaaattcgcccccgaatttgttggcgaggatgctaacaagaaggcagaggagtttctcaaacatagatccttcccttcggaaaaaccactagctggtataccgaacactgccactcatgtcaccaaatataacatgccccctatattgcgtagctcattcaaactcccttccccgagagttgctgcaaatcttactgaaccctctgctccccctaccactccaccacccacacctccccagaacaaggaagagcagcccaaagagtctgatgttgacattgagactatgcatgtctgtaaggttcctgacaatccggaacacagcaagaagccatgctgctcagatgataccgatactaagaaaactaggaagccgatggtcacctttgtggaacccgaggagaaatttgtcggattgggagctagcttgtacagggagaccatgcagacctttgctgctgatggttatgatgaagaaagcaacctatcgtttgaggagactaaccaagagccgggttcttcatctgtagaacaaagactagatagaatagaggagaaattgtcctacataataggccttttaaacaccataatggtagcgactgctggacctaccactgctagagatgagattagagatgcccttataggcactagagaagaacttattgagatgatcaagtctgacatcttgactgtcaatgacagaatagtggccatggagaagctcagagatgaggaatgctccagagctgacactgatgatggatcagcctgttatttaacagacagagcaaggatactagataagatagtgtccagcaatgctgaagaggctaaggaagatttggatgttgatgacatcatgggcattaatttttagttaattaaaataacaacaggacaaataatggaggcctacttggtagagatgtaccatggtgtcccatatacagctgcagtacagctaaacttggttgaaaaacattcagccaacatatcactaactgtgtggataccgatgtttcaaacatctctaccaaagaactccgttatggacctgctacatgatgttacagtcatttgtacacagatatcaacagtgcatggtcccatgatcaaggtagatctgagctcttccaatgcaggtttagctaccatgccaaggcaattcttgataaatgctatcatagctttggatgactggggcaacatggattacgaagtgcctgttgcttttgataaaaagagcttctgtgtgacaattcttaagcctaaaaacatgctttacactgtacccagcattactcccactaatcgacctactcatgagctgatagctgtctgctctttccataacagggtaacattaaagtcattcaatatacctgtcttcatcagagcactgtctatcagacagcaggaccttgatagtgtggagcaggctataagctccgatgtggaccatgctataacaacagctagggtggctccctatgcagggcttacacttgtgatcaacatcacatccaccaaaggagcattcaaactgctaaaggcaggtagtcagattcttgcagaactgggtccctatctgacgcaggtgagcctacatgatgtgattatgaactggaaacatacaggcacttcctacatactcaagagctcctcaacaagtggatgaaaaagagaaagtcaccattgatcagctcaatccacaactacaaccccacgatttcacacagcacaacaaccaccgccaccaacatccacatgacaaccacacatacacacccacatacatatatacacatatcttagttaaataaaatcaggataaataatggatcctaacatgacctcacaccagatcaccctcgagatcaacatgaccagcagccgtattggcacatacactacaccagccccaacagctcttctccttgcatgtgccgtcatcaacacagtgtgtgcgctgataatggcctgcagcagtagaagcactgccacatcaggcattgtcagcagccaatgcacagttcatcccaatcaccctccaccaagttatggcgtcaatgtaactggtctgccgggtaacctatactcaaggaacactacataacattataaataacagaaattatccttcaataaaccccaggccagacagctttaccctgctagacgattcaatcagcccttgcagtatgtcgtctagttaacaaaaaaccggtaggataagtactatcctattggaaccaaacgagacctgtagagcagctcacacaagagaaccacaagctgacttcacctagtatgggaaggaacttagaagtgagtggcagcattaccaatttgaactttgagagaactcagcatcctgacacatttaggactgttgtaaaagtgaaccaaatgtgtaagcttattgcaggtgtgctcacaagtgctgctgtggcagtttgtgtgggggtcataatgtattctgttttcacatcaaaccacaaggccaactccacgcagaatgccacgacccggaacagcacatccacccctccccaaccaaccgccggtctgcccaccacagagcaagggaccatccccagattcaccaaaccccccaccaaaaccgccacccaccatgagatcacagagcccgtcaaaatggcaacaccttcagaggatccctaccaatgctccagcaatggttatttggaccgacctgatttacctgaaaatttcaaactcgtattggatgttatatgcaagcctccaggtcctgaacatcacaacaccagctgttatgagaaacgtgaaatcaacccaggaagtgtttgccctgatcttgtaacaatgaaggcaaacatgggcttaaacaatggtggtggggaggatgctgcaccttatatagaggttaccaccctttctacgtactccaacaaaagggcaatgtgtgtccacaatgggtgtgatcaaggcttctgtttcttcctttctggtttaagcactgatcaggagagagctgtgctagagcttggaggtcaacaggctatcatggagttgcattatgattcctactggaaacactattggagtaactctaattgtgttgttcccagaacaaactgcaacctgacagaccaaactgagattttgtttcctaggtttaacaacaagaatcagtctcagtgtaccacctgtgcagattcagctggcctagataacaaattttatctcacatgtgatgggcttttaagaaccctccctctagttggactacccagcctaagtcctcaggcttacaaagctgtacccacacaaactacaggcaccaccacggcaccaacatcagagacgaggcacccaacccctgcacccaggaggtccaaacctctcagtcggaagaagagagctttatgtggtgtagactcaagcagagaacccaaaccaacaatgccttactggtgtcctatgctccaattatttccaaggaggtctaattcttaagtgacctattcctgaattaacttcagaataagtaccaaccttatcagtagttaatgaaaactaagctttgatataataggacaaatatgattcctggcaggatctttctagtccttctggtgatcttcaacaccaagccaattcacccaaatacattaacagaaaaattctatgagtccacatgtagtgttgagactgcaggttataagagtgcccttagaacaggttggcatatgacagttatgtcaattaagttgtctcaaataaatattgagtcatgcaagagcagcaactcgttattggctcatgagcttgcaatctatagtagtgcagtggatgaattgagaacgttatcatccaatgccttgaagtccaaaaggaagaagaggttcctcggtttgattcttggtctcggagctgcagtcactgccggggtggctttagccaagacagtgcaacttgaaagtgagattgcattgattagagatgcagtgagaaatacaaatgaggctgttgttagcctaaccaacggcatgtcagtgttggctaaagtggtggatgatttgaaaaacttcatatctaaagaattactcccaaaaataaaccgagtctcttgtgatgtgcacgacatcactgccgtcattagattccaacagctcaacaaaagacttttggaagtatctcgtgaattttcatctaatgcaggattaacacacactgtttcatcttttatgttaacagaccgggaactcacctccattgtaggcggcatggctgtttcagcaggccaaaaagagataatgctatctagcaaagctataatgagaagaaatgggttagcaatattaagttcagtcaacgctgacacactggtttatgtaatacaactcccattatttggtgttatggacacagattgttgggtaataagaagttctatagactgtcataacatagcagacaagtatgcttgtttggctagagctgataatggctggtattgtcacaatgctggctcattatcatacttcccgtcgccaacggattgtgagatccacaatgggtatgctttctgtgacactctaaaaagtctaactgtacctgtaacatcacgagaatgcaactcaaacatgtataccactaactacgattgtaagatttccacaagtaaaacttatgtgagtacagcggtactgactacaatgggttgcttggtatcttgttatggtcataacagttgcacagtcatcaataatgacaaaggtataataaggactctgccagatggttgccactacatctccaacaaaggtgtggacagggttcaagtaggtaacactgtttactatcttagcaaagaagttggcaagtcaattgttgtcagaggggaaccattggtcttgaaatatgaccctttgagtttccctgacgataaatttgatgttgctataagagatgtggagcatagcatcaatcagacacgcacattcttgaaggcctctgatcagttattggacttaagtgaaaacagagagaataaaaatttaaacaagtcatatatactaacaactctgctcttcgttgtaatgcttattataataatggctgtcatagggttcattctgtataaggtattgaaaatgatcagagacaacaagttgaaatccaaaagtacacctggcctcacagttttatcatgacaattgtaccaaaccataattgagttagttaattaaaaacttaggataagtgacaatccagacccaacacctctttcaactctcaaggataaggtaggatgagtgtgagaccttgcaaatttgaggttcaagggttttgttccagagggaggaattgcaagtatagtcataaatattgggaatggcctttgaaaactcttatgctcaggcagaactacatgcttaataggatttataggttcctcgacaccaacacagatgcaatgtcagatgtcagcggatttgatgcaccacaaaggactgctgagtatgccttgggaaccataggtgtgctgaaaagttacctggaaaaaactaacaacatcactaaatcaatagcttgtggcagtttgatcactgtattgcagaacttggatgttggtctagtaatacaagcaagagatagcaacactgaggacaccaattacttgagaagttgcaacactatactgtcttatatagacaagatacacaagaagagacaaattattcacattctcaaaagactgccagtaggagtactatgcaatctgatccaatctgtcatctccatcgaggagaagataaattcttctatgaaaacagaatgataaggctgcctaaatactatccagccatactgcataagatgtatattattagagtaaatagaaacctcacttacgatgggtctggaccatccacaataatagatgcaggaaagtctgtggtgtggaatcgtgttgatgtgatagcttgtgtgaaagaggccttgtgctgcatagaattcagctggaataaccaagtgatcatagactttgattatagccaggccagatgatgtggactgtattcctttttttgtcagtaatcagttattaacccaaaattgttaattatgtagactttaagttaactaacttcatgttaattcaatagttatataaaaaaatattcgaattaggatcaatatggatcctattgatgaacaagaagttaatgtgtacttgccggatagctacttaaagggtgttatatcttttagtgaaactaatgctcttggcagctgtatcattggtagacctttcttgaaggatgactttactgccactacttcaatccgtaaccccctaattgaacataaaagaataagggacactaaattagtaaaaaatattgtttcaaaccctcaatataggttagtggagcctctccaaatgcagcatgagctcttgagtgtactatcgcccaatttcatattgcacactgccaacttaaggaaaattatacaaagaagtgttgacataacagataaaaagttgaaccccattttgcacattttgaatcttaattctcctaaccaagagggtaaggtgtcggaacggctaactaggctaattaagaaacatctctctcacatacctaattgggtaagcagctggtacaatatatgggtcaatcttaacaacttactgcaggagtaccgttcaaaggaagttatagaccataactgtgttctgactaggcaattgtctggaagtttcatacatgtggtcatgagtcaatatggagttgtgataattagcaaaaaaagtaaaagatatacaatgtgtacttataaccaattcctaacctggaaggaccttgccttgagcagatttaatgccaattatgtggtctggctaagtaatgtgttaaacacactcaacgaggggttgggattaaggtgtagattaaaaggtcatctgctcagtaagttgtacatttccactgacatctttttatcttcaacatctaatgaattttataatgtggtcaaggaatttgagggcttcatcatgtcactgatattgaaacaaactgaggaagccttatttagcacaaggttttataataacatgttgaacaacttaattgatgccattgatagggctcgactagagtatctggcccgctgtgccaattcagctgccaggattaatttacctagtacagatgttatgatagcatcattgggtgatatcttatctttgataaacgttttaggtgaatccaaccttaacaacttaagtgagttatattttatcttcaggatatttggtcaccctatggttgatgagaggaaggccatggatgcagtcagagataactgttgtgaaacaaagtttctgacggctaagaaccttgcatcgttaagaggagcatatgtttatagaattatcaaaggattcgtagcaaattataacaggtggccttacataaaaactagagtttgccttacaccaacatggattaactatcttgacaccaattcatgtccctcattattagagatgacagaagatgattttattgtgttagctggagtgcactttataagagaattccacatcccaaagctaactgatctggagattatattaaatgacaaggccatatctcctccaaaatcactcatttggtcatgctttccaaaaaactacatacctcaggttatacaagatgagtatgcccggaggtattgtagagctaaagcacctttgaagacaagacgtgtcttggagttctacttacaggacaaggatttcaagttggatcagctccatagagtagtagtgaaccaggactaccttaatgataaagaacatataatttctttaacaggaaaagaaagagagttgggtgttggtaggatgtttgccatgcaacctgggaagcagaggcaagtccaaattttagcagagaagctgttggctgataacatcctgcaattctttccggagacactgactagatacggtgatttggagctgcaaaagatactagagttaaaagctggactttcaaataaaaatgacagatctaaagactcctacaataattatataagtaggtgctcattaattactgatttaagtaaatttaaccaagcttttaggtacgagtcatcctgtgtgtgtagtgatcttttagatgagctacatgggactcaaagcttattctcttggctgcatttaacagtaccactgactactataatgtgtacatataggcatgcgccgccggacactggaaacaactataatgtagatgatattgctgagcagagtggactctaccgctaccacatgggcgggattgagggctggtgccagaagctctggacaacagaggccattgctttgctagatactgtagctgtgaagggccgtttccagctaacttcattaataaatggcgacaaccaaagtattgatatttcaaaaccaacaaggctggggaccaggactcaaagtgaagcagattatgatttggcaataaattctttaagattaatatcagcagcttataaaggcattggacataaattaaaagaaggtgagacctacttgtcacgtgacatgcagttcatgagtaaaacaatacaacatgaaggggtctactacccggcctccatcaagaaaatattaagagttggtccctggatcaacacaatattagatgatataaaaacttcaacagaaagtattggttctctaactcaagaactagaatataaaggtgaaagtttaatgagcagcctgctgctgaggaacttctggctctacagattatattcagtggatttaaaagatcattctttgtgtggaaagcagctctacagatccttaataaaagtgttaaaacatttgaagaggtgcttcaacctggagaaccttggggaatgtttggaattatttttaaatgtgcccatgcagtttggaggtgctgacccaaatgtcatctacaggagcttctacagaagaactccagattttctaacagaaagtataactcatctcatcctcattttaaaacattttagaagagatttggaattcaacaaagataatgtctccaaagctgttctttctttgctagagttcaccaagaatgattctgcagaatttgtaactttgatgagagatcctcaagcaattggtagtgagaggcaggccaagatcacttcagacatcaacagaacagctgtaacttcagtgttatcaaatgctccaaatgaaatatttagaacttcagctcttcattacagcagcacagaaaatgaattaaatggaatagcaagtggagtttctcctgtttatcctcatggtcttcgagttttatatgaaagtttacctttttataaagcagagaagattgtcaacatggtttctgggaccaagtccatcaccaacatactggagaagacatcagccatctcctacacagatataattcgagccaccaacatgatggtggagaacctcactttgctaacaagaataatgaaaccaggtgctgacacatctttggatcctgacacaatagtaataacaatattatcaaaaataataagagataaatcctgggatgttggtgatataattggtgtcacttccccatctcctgtctcctgcttcaaggtggtctacacatcaactctacaaaataattcagtagtaatagaaagatacacaacagacacctacacaagaggtaagagaggccccaccaagccctgggtgggcagcagcacacaggagaagaagtccatgcctgtctacaacagacaagttttaacaagaggacaaagagatcaaatagaaaatatagcaaagctggagtgggtgttttcttcagtagcaaatattgattctttgctaaatgagctcagcaccatgactttgggtctttctctaaggaaatgtagacaactttttccaacatatttaagtttaaacttcctgcacaggctttctgtcagcagcaggcccagagaatatccttcttctcttcctgcctacaggacaacaaattttcattttgatactggaccaataaataaagtgttaacagaaagatttggagatgaagatataaatttggtatttcaaaatgcaatatcatatggtctttccaccatgtctttggtggagcagtttactggtgtctgtccaaataaagttttgctggtgcccaagctacaagaaatacaactaatgaaagttccaatatttcaaggtggcttcaacctacaaagtataattccaataataaggcagcagcacatgttcctgcccaaccacatcactccagcccagtatattgaattatttctttcttcaaaacaatttcattcaagaataaatttaaaacacaacaacagatttaaacttgttttacaaaaagattattttaatggggagaacatgatagaaactttgtccacctgtttggcaggccactggatcatcattttgatgctaatgaaggagagtcaggggatatttgacaaggagtggtatgatggttttgtaacagaccacatgttcctggacctgcagctcttcctctcctccttcaagacatttctaactgtcttcaactttgcttatttaaaagttggttcaaatatagaagaaataacaggaaatcaagccaacctgctggagctgctggacctgggctactggaagaacatgtataaagtattttcagagaccaaggtgcggctggctttgctaaaacaagatttatcatttaattctgtgaagaacagcagcagcttccggcactggtttataaattctctacaagaagtacaatgtacttctgtgccttgggtggtaaatgtaacaagaaatccaactcatttaaaaggtgttctacagtacatgaagatgatagaaagtggcatgattcaaggttattcagcaaatatttcttcagttttaagtatcccatataattatccagacatggcgcacatgatgacaaaaataataagaaatcgaggccacatgtcctatgattatccaaagatgaagaaaagtttaactttctccatgacagacatgagtgacagctacatgctcaacctcttccccaaagtagaatgttcttacatgagtggttatttggataaactagatgatactctacaacttctaaagaaacctcctgttggaagaaaagttccttctgtggctttgccctggcaccactgcaacagatacaactttgtcttcagcagcacaggctgcaaagtttctgtcattgacatgcttccaaaacatttccgaagaagtaatttaaaagtaatatgttttattggagaaggggcgggcaacctcatgctaagagctgttttggaagttggtggaaatataaaattaatatatagatccttaaaagatcctgatgatcatcatgttcctgtagaatttctaagattaaaaccttgttatccttatattgatactggtggcagtttatctttggcttcaacagatgccaccaacaaggcccactgggattatttacatcttcactggacagatcctttaaatttaatagtatgtgatgcagaaataagtggtgtgaagcactggctaaaaattcttcacaggtggtatgagcacatgacttcctgcaagcactgtttaaaatcagaacatgataaatatttaataataaaatatcatgctcaagatgatttaatagatcttcctcatggtgtcaggctactaaaatgtaacatctgcctgggctccaaactaagtggcagtgagagctacctgctcattggtcttggtctttcaaataaacttcctgtctacagtgaagttcttcattcaaaacttcttttggcagaatgtcatcagtttcatcatccaaaatatttggatgtttctggaataaatacaaatataaaatccttaataccaatgttggattatccaataacttacaacaagatcaccactttgctagaaagtgtgcgggagctttcttcaaataaaaacaagaacaccatgtggattggaagaaatcctgtctaccataataaatggctaaagaggaaatatttcaacattttaaaatggctaaaatactgtatagaacttcctgccttcaggatggattataattcatttgagagaatagaaatgctttatccaaatttaagagatttggtagattctgtttctacttcagagctgaagaaagtaataaaagtaacaggcatcctcttcaggagcaacaccatgtgaattaatatgccaatgctaacatgtcatccccactactttttttgttcaatcactctcagtctagacatgtgagaagatcccaaacccaactgcacccctcccactcgaaatcagttgtagcctcagcagctccatctgccatgatctctgaccagcagcaaatgtattattattttaatttgtctatagttaacaaaaaattgatatctcacaggttgtaaacatagttcttttataattattgttagttaaactattgtgtttgacttcctttgggtatttttttcccgt

SEQ ID NO: 61 - DNA, 16655 bp, synthetic construct
  misc_feature 1..16655: note = Chimeric Vector
  source 1..16655: mol_type = other DNA
acgcgaaaaaatgcataacaaaactatcaacctgaaaaaagttaggacaagtgaagattgctagtcccaaagaccagaatcatgggctgtaatgtgatgatggagcttgattatggtggacgagctgcatggctggcattccacataaccaactttgataggtcagatttggaaactatcctaagaggtgctagggtgtgcaatacatggcaagatcagagactctccgtgtaccttgtgggaagggattgcaatttgttaagaccatttgtgcaagctgccaaatttatccataatactaggagaggccaaacattaacacattggttcacaaaaaatatcgtgtttagttctacagggcaagagacagagcctcccattgaccccacatgtgagctgttggtagagctgatcagtggttaagatgacagcacattatgtagttaattaaaacaaagggtaggacaagtcctaatgtccacagctatgaacaagttcactcagaccatctctaaacctgccactatcttgaatatttcagacagtgaagagtcgggcgatgaggcaggggtgggcaaggtgtcccgtaccacacagagctcagagaggtggcttgatttgctcattgaaaagttccagccgagcctacaaaacatcactagatacatcaactggaacttcatcaggatctgcaatgataggcttaaaaaagaaaaaatggggtacattgaggccaagcaatatgtggaagacatggcttggatggtgatagcatctgaggcagacagcattgagtggaagtgcataaggaggcaggagaaagtgactggggtgaaatacccaaagttcttctttgtacaacacaaagaggactggattgagtgcacaggatgcattccgtacccaggccatgacttgatctatgatgaggatgatgatgactgagctgactcagatcactccaaccagcagagagatccatagttatagaaaaacattaggataaatacacatcctaggccgccaccatggaactgctgatcctgaaagccaacgctattactactatcctgaccgccgtgacattttgcttcgcatctggacagaacattactgaggaattctaccagtcaacatgcagcgccgtgtccaaaggatacctgagcgccctgcggaccggctggtatacatcagtgattactatcgagctgtccaacatcaaggaaaacaaatgtaatgggaccgacgcaaaggtgaaactgatcaagcaggagctggataagtacaaaaatgccgtgacagaactgcagctgctgatgcagtccacaccagcaactaacaatcgcgcccggagagagctgccccggttcatgaactataccctgaacaatgctaagaaaaccaatgtgacactgtccaagaaacgcaagaggcgcttcctgggatttctgctgggcgtggggtctgccatcgctagtggagtggccgtctctaaagtcctgcacctggagggcgaagtgaacaagatcaaaagcgccctgctgtccactaacaaggcagtggtcagtctgtcaaatggcgtgtccgtcctgacctctaaggtgctggacctgaaaaattatattgataagcagctgctgcctatcgtcaacaaacagagctgctccatttctaatatcgagacagtgatcgaattccagcagaagaacaatagactgctggagattaccagagagttcagcgtgaacgccggcgtcaccacacccgtgtccacctacatgctgacaaatagtgagctgctgtcactgattaacgacatgcctatcaccaatgatcagaagaaactgatgtccaacaatgtgcagatcgtcagacagcagagttactcaatcatgtctatcattaaggaggaagtcctggcctacgtggtccagctgccactgtatggcgtgatcgacaccccctgctggaaactgcatacatctcctctgtgcactaccaacacaaaggaaggaagtaatatctgcctgactcgaaccgaccggggatggtactgtgataacgcaggcagcgtgtccttctttccacaggccgagacctgcaaggtccagagcaacagggtgttctgtgacactatgaatagcctgaccctgccttccgaagtcaacctgtgcaatgtggacatctttaatccaaagtacgattgtaagatcatgactagcaagaccgatgtcagctcctctgtgattacttctctgggggccatcgtgagttgctacggaaagacaaaatgtactgccagcaacaaaaatcgcggcatcattaagaccttctccaacgggtgcgactatgtctctaacaagggcgtggatacagtgagtgtcgggaacactctgtactatgtcaataagcaggagggaaaaagcctgtacgtgaagggcgaacccatcattaacttctatgaccccctggtgttcccttccgacgagtttgatgcatctattagtcaggtgaacgaaaaaatcaatcagagtctggcctttattcggaagtcagatgagctgctgcacaacgtgaatgctggcaaatctacaactaacatcatgatcaccacaatcatcatcgtgattatcgtcattctgctgtcactgatcgctgtggggctgctgctgtactgtaaggcaagaagcaccccagtcactctgtcaaaagaccagctgtcagggattaacaacattgccttcagtaactgatagccacctatagcaagtgatgtagtatttagtctatgattagttatagaaaaacattaggataaatagccgggccaaaatgtctctagacagattgaagctcaatgatgtctcaaacaaggatagcctgctgtccaactgcaaatacagtgttaccagatccacaggcgatgtaaccagtgtgtctggtcatgctatgcagaaagcccttgcaaggacactcggcatgttcttacttactgccttcaaccgttgcgaagaagtggcagaaatagggctccaatatgccatgtccttgctaggcagagatgatagcatcaagatattaagagaagccggctacaatgtaaaatgtgtggacacacagctcaaggactttacaatcaaattacaaggaaaggaatacaaaatacaagtcctagatatagtgggaatagatgcagccaatttagctgatctagagatacaagccagaggagtggtagcaaaagaactcaaaacaggagccaggctacctgacaatcggaggcatgatgcaccagattgtggtgtgatagttctctgtattgcagcattagttgtttccaaattagctgcaggggacaggggaggacttgatgctgtggaaagaagggctttaaatgtgctgaaagccgagaaagccaggtaccccaacatggaggtcaagcagatagctgaaagtttttatgatctgtttgaaaggaagccttattacattgatgtcttcatcacttttggcctggcccagtctagtgtcaagggaggcagcaaagttgaggggctgttttcaggtctcttcatgaatgcatacggggcaggacaagttatgctgaggtggggtttactggcaaaatctgtcaagaacatcatgctaggccatgctagtgtacaagctgagatggaacaggtggttgaggtttacgaatatgctcagaagcaaggaggggaggcaggattctatcacatcagaaataatccaaaagcttcacttctctctttgaccaattgtcctaatttcaccagtgttgtgcttggcaatgctgcaggtttaggcatcatagggtcatataagggtgctcctaggaatagagaactctttgatgctgccaaagattatgcagaaagattaaaggacaacaatgtaattaactacagtgcattaaacttgactgcagaagaaagagagctgatcagccagcagctgaacattgttgatgacactcctgatgatgatatttaattaaaactggaaaatgtaggataaatatggagaaattcgcccccgaatttgttggcgaggatgctaacaagaaggcagaggagtttctcaaacatagatccttcccttcggaaaaaccactagctggtataccgaacactgccactcatgtcaccaaatataacatgccccctatattgcgtagctcattcaaactcccttccccgagagttgctgcaaatcttactgaaccctctgctccccctaccactccaccacccacacctccccagaacaaggaagagcagcccaaagagtctgatgttgacattgagactatgcatgtctgtaaggttcctgacaatccggaacacagcaagaagccatgctgctcagatgataccgatactaagaaaactaggaagccgatggtcacctttgtggaacccgaggagaaatttgtcggattgggagctagcttgtacagggagaccatgcagacctttgctgctgatggttatgatgaagaaagcaacctatcgtttgaggagactaaccaagagccgggttcttcatctgtagaacaaagactagatagaatagaggagaaattgtcctacataataggccttttaaacaccataatggtagcgactgctggacctaccactgctagagatgagattagagatgcccttataggcactagagaagaacttattgagatgatcaagtctgacatcttgactgtcaatgacagaatagtggccatggagaagctcagagatgaggaatgctccagagctgacactgatgatggatcagcctgttatttaacagacagagcaaggatactagataagatagtgtccagcaatgctgaagaggctaaggaagatttggatgttgatgacatcatgggcattaatttttagttaattaaaataacaacaggacaaataatggaggcctacttggtagagatgtaccatggtgtcccatatacagctgcagtacagctaaacttggttgaaaaacattcagccaacatatcactaactgtgtggataccgatgtttcaaacatctctaccaaagaactccgttatggacctgctacatgatgttacagtcatttgtacacagatatcaacagtgcatggtcccatgatcaaggtagatctgagctcttccaatgcaggtttagctaccatgccaaggcaattcttgataaatgctatcatagctttggatgactggggcaacatggattacgaagtgcctgttgcttttgataaaaagagcttctgtgtgacaattcttaagcctaaaaacatgctttacactgtacccagcattactcccactaatcgacctactcatgagctgatagctgtctgctctttccataacagggtaacattaaagtcattcaatatacctgtcttcatcagagcactgtctatcagacagcaggaccttgatagtgtggagcaggctataagctccgatgtggaccatgctataacaacagctagggtggctccctatgcagggcttacacttgtgatcaacatcacatccaccaaaggagcattcaaactgctaaaggcaggtagtcagattcttgcagaactgggtccctatctgacgcaggtgagcctacatgatgtgattatgaactggaaacatacaggcacttcctacatactcaagagctcctcaacaagtggatgaaaaagagaaagtcaccattgatcagctcaatccacaactacaaccccacgatttcacacagcacaacaaccaccgccaccaacatccacatgacaaccacacatacacacccacatacatatatacacatatcttagttaaataaaatcaggataaataatggatcctaacatgacctcacaccagatcaccctcgagatcaacatgaccagcagccgtattggcacatacactacaccagccccaacagctcttctccttgcatgtgccgtcatcaacacagtgtgtgcgctgataatggcctgcagcagtagaagcactgccacatcaggcattgtcagcagccaatgcacagttcatcccaatcaccctccaccaagttatggcgtcaatgtaactggtctgccgggtaacctatactcaaggaacactacataacattataaataacagaaattatccttcaataaaccccaggccagacagctttaccctgctagacgattcaatcagcccttgcagtatgtcgtctagttaacaaaaaaccggtaggataagtactatcctattggaaccaaacgagacctgtagagcagctcacacaagagaaccacaagctgacttcacctagtatgggaaggaacttagaagtgagtggcagcattaccaatttgaactttgagagaactcagcatcctgacacatttaggactgttgtaaaagtgaaccaaatgtgtaagcttattgcaggtgtgctcacaagtgctgctgtggcagtttgtgtgggggtcataatgtattctgttttcacatcaaaccacaaggccaactccacgcagaatgccacgacccggaacagcacatccacccctccccaaccaaccgccggtctgcccaccacagagcaagggaccatccccagattcaccaaaccccccaccaaaaccgccacccaccatgagatcacagagcccgtcaaaatggcaacaccttcagaggatccctaccaatgctccagcaatggttatttggaccgacctgatttacctgaaaatttcaaactcgtattggatgttatatgcaagcctccaggtcctgaacatcacaacaccagctgttatgagaaacgtgaaatcaacccaggaagtgtttgccctgatcttgtaacaatgaaggcaaacatgggcttaaacaatggtggtggggaggatgctgcaccttatatagaggttaccaccctttctacgtactccaacaaaagggcaatgtgtgtccacaatgggtgtgatcaaggcttctgtttcttcctttctggtttaagcactgatcaggagagagctgtgctagagcttggaggtcaacaggctatcatggagttgcattatgattcctactggaaacactattggagtaactctaattgtgttgttcccagaacaaactgcaacctgacagaccaaactgagattttgtttcctaggtttaacaacaagaatcagtctcagtgtaccacctgtgcagattcagctggcctagataacaaattttatctcacatgtgatgggcttttaagaaccctccctctagttggactacccagcctaagtcctcaggcttacaaagctgtacccacacaaactacaggcaccaccacggcaccaacatcagagacgaggcacccaacccctgcacccaggaggtccaaacctctcagtcggaagaagagagctttatgtggtgtagactcaagcagagaacccaaaccaacaatgccttactggtgtcctatgctccaattatttccaaggaggtctaattcttaagtgacctattcctgaattaacttcagaataagtaccaaccttatcagtagttaatgaaaactaagctttgatataataggacaaatatgattcctggcaggatctttctagtccttctggtgatcttcaacaccaagccaattcacccaaatacattaacagaaaaattctatgagtccacatgtagtgttgagactgcaggttataagagtgcccttagaacaggttggcatatgacagttatgtcaattaagttgtctcaaataaatattgagtcatgcaagagcagcaactcgttattggctcatgagcttgcaatctatagtagtgcagtggatgaattgagaacgttatcatccaatgccttgaagtccaaaaggaagaagaggttcctcggtttgattcttggtctcggagctgcagtcactgccggggtggctttagccaagacagtgcaacttgaaagtgagattgcattgattagagatgcagtgagaaatacaaatgaggctgttgttagcctaaccaacggcatgtcagtgttggctaaagtggtggatgatttgaaaaacttcatatctaaagaattactcccaaaaataaaccgagtctcttgtgatgtgcacgacatcactgccgtcattagattccaacagctcaacaaaagacttttggaagtatctcgtgaattttcatctaatgcaggattaacacacactgtttcatcttttatgttaacagaccgggaactcacctccattgtaggcggcatggctgtttcagcaggccaaaaagagataatgctatctagcaaagctataatgagaagaaatgggttagcaatattaagttcagtcaacgctgacacactggtttatgtaatacaactcccattatttggtgttatggacacagattgttgggtaataagaagttctatagactgtcataacatagcagacaagtatgcttgtttggctagagctgataatggctggtattgtcacaatgctggctcattatcatacttcccgtcgccaacggattgtgagatccacaatgggtatgctttctgtgacactctaaaaagtctaactgtacctgtaacatcacgagaatgcaactcaaacatgtataccactaactacgattgtaagatttccacaagtaaaacttatgtgagtacagcggtactgactacaatgggttgcttggtatcttgttatggtcataacagttgcacagtcatcaataatgacaaaggtataataaggactctgccagatggttgccactacatctccaacaaaggtgtggacagggttcaagtaggtaacactgtttactatcttagcaaagaagttggcaagtcaattgttgtcagaggggaaccattggtcttgaaatatgaccctttgagtttccctgacgataaatttgatgttgctataagagatgtggagcatagcatcaatcagacacgcacattcttgaaggcctctgatcagttattggacttaagtgaaaacagagagaataaaaatttaaacaagtcatatatactaacaactctgctcttcgttgtaatgcttattataataatggctgtcatagggttcattctgtataaggtattgaaaatgatcagagacaacaagttgaaatccaaaagtacacctggcctcacagttttatcatgacaattgtaccaaaccataattgagttagttaattaaaaacttaggataagtgacaatccagacccaacacctctttcaactctcaaggataaggtaggatgagtgtgagaccttgcaaatttgaggttcaagggttttgttccagagggaggaattgcaagtatagtcataaatattgggaatggcctttgaaaactcttatgctcaggcagaactacatgcttaataggatttataggttcctcgacaccaacacagatgcaatgtcagatgtcagcggatttgatgcaccacaaaggactgctgagtatgccttgggaaccataggtgtgctgaaaagttacctggaaaaaactaacaacatcactaaatcaatagcttgtggcagtttgatcactgtattgcagaacttggatgttggtctagtaatacaagcaagagatagcaacactgaggacaccaattacttgagaagttgcaacactatactgtcttatatagacaagatacacaagaagagacaaattattcacattctcaaaagactgccagtaggagtactatgcaatctgatccaatctgtcatctccatcgaggagaagataaattcttctatgaaaacagaatgataaggctgcctaaatactatccagccatactgcataagatgtatattattagagtaaatagaaacctcacttacgatgggtctggaccatccacaataatagatgcaggaaagtctgtggtgtggaatcgtgttgatgtgatagcttgtgtgaaagaggccttgtgctgcatagaattcagctggaataaccaagtgatcatagactttgattatagccaggccagatgatgtggactgtattcctttttttgtcagtaatcagttattaacccaaaattgttaattatgtagactttaagttaactaacttcatgttaattcaatagttatataaaaaaatattcgaattaggatcaatatggatcctattgatgaacaagaagttaatgtgtacttgccggatagctacttaaagggtgttatatcttttagtgaaactaatgctcttggcagctgtatcattggtagacctttcttgaaggatgactttactgccactacttcaatccgtaaccccctaattgaacataaaagaataagggacactaaattagtaaaaaatattgtttcaaaccctcaatataggttagtggagcctctccaaatgcagcatgagctcttgagtgtactatcgcccaatttcatattgcacactgccaacttaaggaaaattatacaaagaagtgttgacataacagataaaaagttgaaccccattttgcacattttgaatcttaattctcctaaccaagagggtaaggtgtcggaacggctaactaggctaattaagaaacatctctctcacatacctaattgggtaagcagctggtacaatatatgggtcaatcttaacaacttactgcaggagtaccgttcaaaggaagttatagaccataactgtgttctgactaggcaattgtctggaagtttcatacatgtggtcatgagtcaatatggagttgtgataattagcaaaaaaagtaaaagatatacaatgtgtacttataaccaattcctaacctggaaggaccttgccttgagcagatttaatgccaattatgtggtctggctaagtaatgtgttaaacacactcaacgaggggttgggattaaggtgtagattaaaaggtcatctgctcagtaagttgtacatttccactgacatctttttatcttcaacatctaatgaattttataatgtggtcaaggaatttgagggcttcatcatgtcactgatattgaaacaaactgaggaagccttatttagcacaaggttttataataacatgttgaacaacttaattgatgccattgatagggctcgactagagtatctggcccgctgtgccaattcagctgccaggattaatttacctagtacagatgttatgatagcatcattgggtgatatcttatctttgataaacgttttaggtgaatccaaccttaacaacttaagtgagttatattttatcttcaggatatttggtcaccctatggttgatgagaggaaggccatggatgcagtcagagataactgttgtgaaacaaagtttctgacggctaagaaccttgcatcgttaagaggagcatatgtttatagaattatcaaaggattcgtagcaaattataacaggtggccttacataaaaactagagtttgccttacaccaacatggattaactatcttgacaccaattcatgtccctcattattagagatgacagaagatgattttattgtgttagctggagtgcactttataagagaattccacatcccaaagctaactgatctggagattatattaaatgacaaggccatatctcctccaaaatcactcatttggtcatgctttccaaaaaactacatacctcaggttatacaagatgagtatgcccggaggtattgtagagctaaagcacctttgaagacaagacgtgtcttggagttctacttacaggacaaggatttcaagttggatcagctccatagagtagtagtgaaccaggactaccttaatgataaagaacatataatttctttaacaggaaaagaaagagagttgggtgttggtaggatgtttgccatgcaacctgggaagcagaggcaagtccaaattttagcagagaagctgttggctgataacatcctgcaattctttccggagacactgactagatacggtgatttggagctgcaaaagatactagagttaaaagctggactttcaaataaaaatgacagatctaaagactcctacaataattatataagtaggtgctcattaattactgatttaagtaaatttaaccaagcttttaggtacgagtcatcctgtgtgtgtagtgatcttttagatgagctacatgggactcaaagcttattctcttggctgcatttaacagtaccactgactactataatgtgtacatataggcatgcgccgccggacactggaaacaactataatgtagatgatattgctgagcagagtggactctaccgctaccacatgggcgggattgagggctggtgccagaagctctggacaacagaggccattgctttgctagatactgtagctgtgaagggccgtttccagctaacttcattaataaatggcgacaaccaaagtattgatatttcaaaaccaacaaggctggggaccaggactcaaagtgaagcagattatgatttggcaataaattctttaagattaatatcagcagcttataaaggcattggacataaattaaaagaaggtgagacctacttgtcacgtgacatgcagttcatgagtaaaacaatacaacatgaaggggtctactacccggcctccatcaagaaaatattaagagttggtccctggatcaacacaatattagatgatataaaaacttcaacagaaagtattggttctctaactcaagaactagaatataaaggtgaaagtttaatgagcagcctgctgctgaggaacttctggctctacagattatattcagtggatttaaaagatcattctttgtgtggaaagcagctctacagatccttaataaaagtgttaaaacatttgaagaggtgcttcaacctggagaaccttggggaatgtttggaattatttttaaatgtgcccatgcagtttggaggtgctgacccaaatgtcatctacaggagcttctacagaagaactccagattttctaacagaaagtataactcatctcatcctcattttaaaacattttagaagagatttggaattcaacaaagataatgtctccaaagctgttctttctttgctagagttcaccaagaatgattctgcagaatttgtaactttgatgagagatcctcaagcaattggtagtgagaggcaggccaagatcacttcagacatcaacagaacagctgtaacttcagtgttatcaaatgctccaaatgaaatatttagaacttcagctcttcattacagcagcacagaaaatgaattaaatggaatagcaagtggagtttctcctgtttatcctcatggtcttcgagttttatatgaaagtttacctttttataaagcagagaagattgtcaacatggtttctgggaccaagtccatcaccaacatactggagaagacatcagccatctcctacacagatataattcgagccaccaacatgatggtggagaacctcactttgctaacaagaataatgaaaccaggtgctgacacatctttggatcctgacacaatagtaataacaatattatcaaaaataataagagataaatcctgggatgttggtgatataattggtgtcacttccccatctcctgtctcctgcttcaaggtggtctacacatcaactctacaaaataattcagtagtaatagaaagatacacaacagacacctacacaagaggtaagagaggccccaccaagccctgggtgggcagcagcacacaggagaagaagtccatgcctgtctacaacagacaagttttaacaagaggacaaagagatcaaatagaaaatatagcaaagctggagtgggtgttttcttcagtagcaaatattgattctttgctaaatgagctcagcaccatgactttgggtctttctctaaggaaatgtagacaactttttccaacatatttaagtttaaacttcctgcacaggctttctgtcagcagcaggcccagagaatatccttcttctcttcctgcctacaggacaacaaattttcattttgatactggaccaataaataaagtgttaacagaaagatttggagatgaagatataaatttggtatttcaaaatgcaatatcatatggtctttccaccatgtctttggtggagcagtttactggtgtctgtccaaataaagttttgctggtgcccaagctacaagaaatacaactaatgaaagttccaatatttcaaggtggcttcaacctacaaagtataattccaataataaggcagcagcacatgttcctgcccaaccacatcactccagcccagtatattgaattatttctttcttcaaaacaatttcattcaagaataaatttaaaacacaacaacagatttaaacttgttttacaaaaagattattttaatggggagaacatgatagaaactttgtccacctgtttggcaggccactggatcatcattttgatgctaatgaaggagagtcaggggatatttgacaaggagtggtatgatggttttgtaacagaccacatgttcctggacctgcagctcttcctctcctccttcaagacatttctaactgtcttcaactttgcttatttaaaagttggttcaaatatagaagaaataacaggaaatcaagccaacctgctggagctgctggacctgggctactggaagaacatgtataaagtattttcagagaccaaggtgcggctggctttgctaaaacaagatttatcatttaattctgtgaagaacagcagcagcttccggcactggtttataaattctctacaagaagtacaatgtacttctgtgccttgggtggtaaatgtaacaagaaatccaactcatttaaaaggtgttctacagtacatgaagatgatagaaagtggcatgattcaaggttattcagcaaatatttcttcagttttaagtatcccatataattatccagacatggcgcacatgatgacaaaaataataagaaatcgaggccacatgtcctatgattatccaaagatgaagaaaagtttaactttctccatgacagacatgagtgacagctacatgctcaacctcttccccaaagtagaatgttcttacatgagtggttatttggataaactagatgatactctacaacttctaaagaaacctcctgttggaagaaaagttccttctgtggctttgccctggcaccactgcaacagatacaactttgtcttcagcagcacaggctgcaaagtttctgtcattgacatgcttccaaaacatttccgaagaagtaatttaaaagtaatatgttttattggagaaggggcgggcaacctcatgctaagagctgttttggaagttggtggaaatataaaattaatatatagatccttaaaagatcctgatgatcatcatgttcctgtagaatttctaagattaaaaccttgttatccttatattgatactggtggcagtttatctttggcttcaacagatgccaccaacaaggcccactgggattatttacatcttcactggacagatcctttaaatttaatagtatgtgatgcagaaataagtggtgtgaagcactggctaaaaattcttcacaggtggtatgagcacatgacttcctgcaagcactgtttaaaatcagaacatgataaatatttaataataaaatatcatgctcaagatgatttaatagatcttcctcatggtgtcaggctactaaaatgtaacatctgcctgggctccaaactaagtggcagtgagagctacctgctcattggtcttggtctttcaaataaacttcctgtctacagtgaagttcttcattcaaaacttcttttggcagaatgtcatcagtttcatcatccaaaatatttggatgtttctggaataaatacaaatataaaatccttaataccaatgttggattatccaataacttacaacaagatcaccactttgctagaaagtgtgcgggagctttcttcaaataaaaacaagaacaccatgtggattggaagaaatcctgtctaccataataaatggctaaagaggaaatatttcaacattttaaaatggctaaaatactgtatagaacttcctgccttcaggatggattataattcatttgagagaatagaaatgctttatccaaatttaagagatttggtagattctgtttctacttcagagctgaagaaagtaataaaagtaacaggcatcctcttcaggagcaacaccatgtgaattaatatgccaatgctaacatgtcatccccactactttttttgttcaatcactctcagtctagacatgtgagaagatcccaaacccaactgcacccctcccactcgaaatcagttgtagcctcagcagctccatctgccatgatctctgaccagcagcaaatgtattattattttaatttgtctatagttaacaaaaaattgatatctcacaggttgtaaacatagttcttttataattattgttagttaaactattgtgtttgacttcctttgggtatttttttcccgt

SEQ ID NO: 62 - DNA, 16661 bp, synthetic construct
  misc_feature 1..16661: note = Chimeric Vector
  source 1..16661: mol_type = other DNA
acgcgaaaaaatgcataacaaaactatcaacctgaaaaaagttaggacaagtgaagattgctagtcccaaagaccagaatcatgggctgtaatgtgatgatggagcttgattatggtggacgagctgcatggctggcattccacataaccaactttgataggtcagatttggaaactatcctaagaggtgctagggtgtgcaatacatggcaagatcagagactctccgtgtaccttgtgggaagggattgcaatttgttaagaccatttgtgcaagctgccaaatttatccataatactaggagaggccaaacattaacacattggttcacaaaaaatatcgtgtttagttctacagggcaagagacagagcctcccattgaccccacatgtgagctgttggtagagctgatcagtggttaagatgacagcacattatgtagttaattaaaacaaagggtaggacaagtcctaatgtccacagctatgaacaagttcactcagaccatctctaaacctgccactatcttgaatatttcagacagtgaagagtcgggcgatgaggcaggggtgggcaaggtgtcccgtaccacacagagctcagagaggtggcttgatttgctcattgaaaagttccagccgagcctacaaaacatcactagatacatcaactggaacttcatcaggatctgcaatgataggcttaaaaaagaaaaaatggggtacattgaggccaagcaatatgtggaagacatggcttggatggtgatagcatctgaggcagacagcattgagtggaagtgcataaggaggcaggagaaagtgactggggtgaaatacccaaagttcttctttgtacaacacaaagaggactggattgagtgcacaggatgcattccgtacccaggccatgacttgatctatgatgaggatgatgatgactgagctgactcagatcactccaaccagcagagagatccaccacctatagcaagtgatgtagtatttagtctatgattagttatagaaaaacattaggataaatacacatcctaggccgggccaaaatgtctctagacagattgaagctcaatgatgtctcaaacaaggatagcctgctgtccaactgcaaatacagtgttaccagatccacaggcgatgtaaccagtgtgtctggtcatgctatgcagaaagcccttgcaaggacactcggcatgttcttacttactgccttcaaccgttgcgaagaagtggcagaaatagggctccaatatgccatgtccttgctaggcagagatgatagcatcaagatattaagagaagccggctacaatgtaaaatgtgtggacacacagctcaaggactttacaatcaaattacaaggaaaggaatacaaaatacaagtcctagatatagtgggaatagatgcagccaatttagctgatctagagatacaagccagaggagtggtagcaaaagaactcaaaacaggagccaggctacctgacaatcggaggcatgatgcaccagattgtggtgtgatagttctctgtattgcagcattagttgtttccaaattagctgcaggggacaggggaggacttgatgctgtggaaagaagggctttaaatgtgctgaaagccgagaaagccaggtaccccaacatggaggtcaagcagatagctgaaagtttttatgatctgtttgaaaggaagccttattacattgatgtcttcatcacttttggcctggcccagtctagtgtcaagggaggcagcaaagttgaggggctgttttcaggtctcttcatgaatgcatacggggcaggacaagttatgctgaggtggggtttactggcaaaatctgtcaagaacatcatgctaggccatgctagtgtacaagctgagatggaacaggtggttgaggtttacgaatatgctcagaagcaaggaggggaggcaggattctatcacatcagaaataatccaaaagcttcacttctctctttgaccaattgtcctaatttcaccagtgttgtgcttggcaatgctgcaggtttaggcatcatagggtcatataagggtgctcctaggaatagagaactctttgatgctgccaaagattatgcagaaagattaaaggacaacaatgtaattaactacagtgcattaaacttgactgcagaagaaagagagctgatcagccagcagctgaacattgttgatgacactcctgatgatgatatttaattaaaactggaaaatgtaggataaatagccgccaccatggaactgctgatcctgaaagccaacgctattactactatcctgaccgccgtgacattttgcttcgcatctggacagaacattactgaggaattctaccagtcaacatgcagcgccgtgtccaaaggatacctgagcgccctgcggaccggctggtatacatcagtgattactatcgagctgtccaacatcaaggaaaacaaatgtaatgggaccgacgcaaaggtgaaactgatcaagcaggagctggataagtacaaaaatgccgtgacagaactgcagctgctgatgcagtccacaccagcaactaacaatcgcgcccggagagagctgccccggttcatgaactataccctgaacaatgctaagaaaaccaatgtgacactgtccaagaaacgcaagaggcgcttcctgggatttctgctgggcgtggggtctgccatcgctagtggagtggccgtctctaaagtcctgcacctggagggcgaagtgaacaagatcaaaagcgccctgctgtccactaacaaggcagtggtcagtctgtcaaatggcgtgtccgtcctgacctctaaggtgctggacctgaaaaattatattgataagcagctgctgcctatcgtcaacaaacagagctgctccatttctaatatcgagacagtgatcgaattccagcagaagaacaatagactgctggagattaccagagagttcagcgtgaacgccggcgtcaccacacccgtgtccacctacatgctgacaaatagtgagctgctgtcactgattaacgacatgcctatcaccaatgatcagaagaaactgatgtccaacaatgtgcagatcgtcagacagcagagttactcaatcatgtctatcattaaggaggaagtcctggcctacgtggtccagctgccactgtatggcgtgatcgacaccccctgctggaaactgcatacatctcctctgtgcactaccaacacaaaggaaggaagtaatatctgcctgactcgaaccgaccggggatggtactgtgataacgcaggcagcgtgtccttctttccacaggccgagacctgcaaggtccagagcaacagggtgttctgtgacactatgaatagcctgaccctgccttccgaagtcaacctgtgcaatgtggacatctttaatccaaagtacgattgtaagatcatgactagcaagaccgatgtcagctcctctgtgattacttctctgggggccatcgtgagttgctacggaaagacaaaatgtactgccagcaacaaaaatcgcggcatcattaagaccttctccaacgggtgcgactatgtctctaacaagggcgtggatacagtgagtgtcgggaacactctgtactatgtcaataagcaggagggaaaaagcctgtacgtgaagggcgaacccatcattaacttctatgaccccctggtgttcccttccgacgagtttgatgcatctattagtcaggtgaacgaaaaaatcaatcagagtctggcctttattcggaagtcagatgagctgctgcacaacgtgaatgctggcaaatctacaactaacatcatgatcaccacaatcatcatcgtgattatcgtcattctgctgtcactgatcgctgtggggctgctgctgtactgtaaggcaagaagcaccccagtcactctgtcaaaagaccagctgtcagggattaacaacattgccttcagtaactgatagtatttaattaaaactggaaaatgtaggataaatatggagaaattcgcccccgaatttgttggcgaggatgctaacaagaaggcagaggagtttctcaaacatagatccttcccttcggaaaaaccactagctggtataccgaacactgccactcatgtcaccaaatataacatgccccctatattgcgtagctcattcaaactcccttccccgagagttgctgcaaatcttactgaaccctctgctccccctaccactccaccacccacacctccccagaacaaggaagagcagcccaaagagtctgatgttgacattgagactatgcatgtctgtaaggttcctgacaatccggaacacagcaagaagccatgctgctcagatgataccgatactaagaaaactaggaagccgatggtcacctttgtggaacccgaggagaaatttgtcggattgggagctagcttgtacagggagaccatgcagacctttgctgctgatggttatgatgaagaaagcaacctatcgtttgaggagactaaccaagagccgggttcttcatctgtagaacaaagactagatagaatagaggagaaattgtcctacataataggccttttaaacaccataatggtagcgactgctggacctaccactgctagagatgagattagagatgcccttataggcactagagaagaacttattgagatgatcaagtctgacatcttgactgtcaatgacagaatagtggccatggagaagctcagagatgaggaatgctccagagctgacactgatgatggatcagcctgttatttaacagacagagcaaggatactagataagatagtgtccagcaatgctgaagaggctaaggaagatttggatgttgatgacatcatgggcattaatttttagttaattaaaataacaacaggacaaataatggaggcctacttggtagagatgtaccatggtgtcccatatacagctgcagtacagctaaacttggttgaaaaacattcagccaacatatcactaactgtgtggataccgatgtttcaaacatctctaccaaagaactccgttatggacctgctacatgatgttacagtcatttgtacacagatatcaacagtgcatggtcccatgatcaaggtagatctgagctcttccaatgcaggtttagctaccatgccaaggcaattcttgataaatgctatcatagctttggatgactggggcaacatggattacgaagtgcctgttgcttttgataaaaagagcttctgtgtgacaattcttaagcctaaaaacatgctttacactgtacccagcattactcccactaatcgacctactcatgagctgatagctgtctgctctttccataacagggtaacattaaagtcattcaatatacctgtcttcatcagagcactgtctatcagacagcaggaccttgatagtgtggagcaggctataagctccgatgtggaccatgctataacaacagctagggtggctccctatgcagggcttacacttgtgatcaacatcacatccaccaaaggagcattcaaactgctaaaggcaggtagtcagattcttgcagaactgggtccctatctgacgcaggtgagcctacatgatgtgattatgaactggaaacatacaggcacttcctacatactcaagagctcctcaacaagtggatgaaaaagagaaagtcaccattgatcagctcaatccacaactacaaccccacgatttcacacagcacaacaaccaccgccaccaacatccacatgacaaccacacatacacacccacatacatatatacacatatcttagttaaataaaatcaggataaataatggatcctaacatgacctcacaccagatcaccctcgagatcaacatgaccagcagccgtattggcacatacactacaccagccccaacagctcttctccttgcatgtgccgtcatcaacacagtgtgtgcgctgataatggcctgcagcagtagaagcactgccacatcaggcattgtcagcagccaatgcacagttcatcccaatcaccctccaccaagttatggcgtcaatgtaactggtctgccgggtaacctatactcaaggaacactacataacattataaataacagaaattatccttcaataaaccccaggccagacagctttaccctgctagacgattcaatcagcccttgcagtatgtcgtctagttaacaaaaaaccggtaggataagtactatcctattggaaccaaacgagacctgtagagcagctcacacaagagaaccacaagctgacttcacctagtatgggaaggaacttagaagtgagtggcagcattaccaatttgaactttgagagaactcagcatcctgacacatttaggactgttgtaaaagtgaaccaaatgtgtaagcttattgcaggtgtgctcacaagtgctgctgtggcagtttgtgtgggggtcataatgtattctgttttcacatcaaaccacaaggccaactccacgcagaatgccacgacccggaacagcacatccacccctccccaaccaaccgccggtctgcccaccacagagcaagggaccatccccagattcaccaaaccccccaccaaaaccgccacccaccatgagatcacagagcccgtcaaaatggcaacaccttcagaggatccctaccaatgctccagcaatggttatttggaccgacctgatttacctgaaaatttcaaactcgtattggatgttatatgcaagcctccaggtcctgaacatcacaacaccagctgttatgagaaacgtgaaatcaacccaggaagtgtttgccctgatcttgtaacaatgaaggcaaacatgggcttaaacaatggtggtggggaggatgctgcaccttatatagaggttaccaccctttctacgtactccaacaaaagggcaatgtgtgtccacaatgggtgtgatcaaggcttctgtttcttcctttctggtttaagcactgatcaggagagagctgtgctagagcttggaggtcaacaggctatcatggagttgcattatgattcctactggaaacactattggagtaactctaattgtgttgttcccagaacaaactgcaacctgacagaccaaactgagattttgtttcctaggtttaacaacaagaatcagtctcagtgtaccacctgtgcagattcagctggcctagataacaaattttatctcacatgtgatgggcttttaagaaccctccctctagttggactacccagcctaagtcctcaggcttacaaagctgtacccacacaaactacaggcaccaccacggcaccaacatcagagacgaggcacccaacccctgcacccaggaggtccaaacctctcagtcggaagaagagagctttatgtggtgtagactcaagcagagaacccaaaccaacaatgccttactggtgtcctatgctccaattatttccaaggaggtctaattcttaagtgacctattcctgaattaacttcagaataagtaccaaccttatcagtagttaatgaaaactaagctttgatataataggacaaatatgattcctggcaggatctttctagtccttctggtgatcttcaacaccaagccaattcacccaaatacattaacagaaaaattctatgagtccacatgtagtgttgagactgcaggttataagagtgcccttagaacaggttggcatatgacagttatgtcaattaagttgtctcaaataaatattgagtcatgcaagagcagcaactcgttattggctcatgagcttgcaatctatagtagtgcagtggatgaattgagaacgttatcatccaatgccttgaagtccaaaaggaagaagaggttcctcggtttgattcttggtctcggagctgcagtcactgccggggtggctttagccaagacagtgcaacttgaaagtgagattgcattgattagagatgcagtgagaaatacaaatgaggctgttgttagcctaaccaacggcatgtcagtgttggctaaagtggtggatgatttgaaaaacttcatatctaaagaattactcccaaaaataaaccgagtctcttgtgatgtgcacgacatcactgccgtcattagattccaacagctcaacaaaagacttttggaagtatctcgtgaattttcatctaatgcaggattaacacacactgtttcatcttttatgttaacagaccgggaactcacctccattgtaggcggcatggctgtttcagcaggccaaaaagagataatgctatctagcaaagctataatgagaagaaatgggttagcaatattaagttcagtcaacgctgacacactggtttatgtaatacaactcccattatttggtgttatggacacagattgttgggtaataagaagttctatagactgtcataacatagcagacaagtatgcttgtttggctagagctgataatggctggtattgtcacaatgctggctcattatcatacttcccgtcgccaacggattgtgagatccacaatgggtatgctttctgtgacactctaaaaagtctaactgtacctgtaacatcacgagaatgcaactcaaacatgtataccactaactacgattgtaagatttccacaagtaaaacttatgtgagtacagcggtactgactacaatgggttgcttggtatcttgttatggtcataacagttgcacagtcatcaataatgacaaaggtataataaggactctgccagatggttgccactacatctccaacaaaggtgtggacagggttcaagtaggtaacactgtttactatcttagcaaagaagttggcaagtcaattgttgtcagaggggaaccattggtcttgaaatatgaccctttgagtttccctgacgataaatttgatgttgctataagagatgtggagcatagcatcaatcagacacgcacattcttgaaggcctctgatcagttattggacttaagtgaaaacagagagaataaaaatttaaacaagtcatatatactaacaactctgctcttcgttgtaatgcttattataataatggctgtcatagggttcattctgtataaggtattgaaaatgatcagagacaacaagttgaaatccaaaagtacacctggcctcacagttttatcatgacaattgtaccaaaccataattgagttagttaattaaaaacttaggataagtgacaatccagacccaacacctctttcaactctcaaggataaggtaggatgagtgtgagaccttgcaaatttgaggttcaagggttttgttccagagggaggaattgcaagtatagtcataaatattgggaatggcctttgaaaactcttatgctcaggcagaactacatgcttaataggatttataggttcctcgacaccaacacagatgcaatgtcagatgtcagcggatttgatgcaccacaaaggactgctgagtatgccttgggaaccataggtgtgctgaaaagttacctggaaaaaactaacaacatcactaaatcaatagcttgtggcagtttgatcactgtattgcagaacttggatgttggtctagtaatacaagcaagagatagcaacactgaggacaccaattacttgagaagttgcaacactatactgtcttatatagacaagatacacaagaagagacaaattattcacattctcaaaagactgccagtaggagtactatgcaatctgatccaatctgtcatctccatcgaggagaagataaattcttctatgaaaacagaatgataaggctgcctaaatactatccagccatactgcataagatgtatattattagagtaaatagaaacctcacttacgatgggtctggaccatccacaataatagatgcaggaaagtctgtggtgtggaatcgtgttgatgtgatagcttgtgtgaaagaggccttgtgctgcatagaattcagctggaataaccaagtgatcatagactttgattatagccaggccagatgatgtggactgtattcctttttttgtcagtaatcagttattaacccaaaattgttaattatgtagactttaagttaactaacttcatgttaattcaatagttatataaaaaaatattcgaattaggatcaatatggatcctattgatgaacaagaagttaatgtgtacttgccggatagctacttaaagggtgttatatcttttagtgaaactaatgctcttggcagctgtatcattggtagacctttcttgaaggatgactttactgccactacttcaatccgtaaccccctaattgaacataaaagaataagggacactaaattagtaaaaaatattgtttcaaaccctcaatataggttagtggagcctctccaaatgcagcatgagctcttgagtgtactatcgcccaatttcatattgcacactgccaacttaaggaaaattatacaaagaagtgttgacataacagataaaaagttgaaccccattttgcacattttgaatcttaattctcctaaccaagagggtaaggtgtcggaacggctaactaggctaattaagaaacatctctctcacatacctaattgggtaagcagctggtacaatatatgggtcaatcttaacaacttactgcaggagtaccgttcaaaggaagttatagaccataactgtgttctgactaggcaattgtctggaagtttcatacatgtggtcatgagtcaatatggagttgtgataattagcaaaaaaagtaaaagatatacaatgtgtacttataaccaattcctaacctggaaggaccttgccttgagcagatttaatgccaattatgtggtctggctaagtaatgtgttaaacacactcaacgaggggttgggattaaggtgtagattaaaaggtcatctgctcagtaagttgtacatttccactgacatctttttatcttcaacatctaatgaattttataatgtggtcaaggaatttgagggcttcatcatgtcactgatattgaaacaaactgaggaagccttatttagcacaaggttttataataacatgttgaacaacttaattgatgccattgatagggctcgactagagtatctggcccgctgtgccaattcagctgccaggattaatttacctagtacagatgttatgatagcatcattgggtgatatcttatctttgataaacgttttaggtgaatccaaccttaacaacttaagtgagttatattttatcttcaggatatttggtcaccctatggttgatgagaggaaggccatggatgcagtcagagataactgttgtgaaacaaagtttctgacggctaagaaccttgcatcgttaagaggagcatatgtttatagaattatcaaaggattcgtagcaaattataacaggtggccttacataaaaactagagtttgccttacaccaacatggattaactatcttgacaccaattcatgtccctcattattagagatgacagaagatgattttattgtgttagctggagtgcactttataagagaattccacatcccaaagctaactgatctggagattatattaaatgacaaggccatatctcctccaaaatcactcatttggtcatgctttccaaaaaactacatacctcaggttatacaagatgagtatgcccggaggtattgtagagctaaagcacctttgaagacaagacgtgtcttggagttctacttacaggacaaggatttcaagttggatcagctccatagagtagtagtgaaccaggactaccttaatgataaagaacatataatttctttaacaggaaaagaaagagagttgggtgttggtaggatgtttgccatgcaacctgggaagcagaggcaagtccaaattttagcagagaagctgttggctgataacatcctgcaattctttccggagacactgactagatacggtgatttggagctgcaaaagatactagagttaaaagctggactttcaaataaaaatgacagatctaaagactcctacaataattatataagtaggtgctcattaattactgatttaagtaaatttaaccaagcttttaggtacgagtcatcctgtgtgtgtagtgatcttttagatgagctacatgggactcaaagcttattctcttggctgcatttaacagtaccactgactactataatgtgtacatataggcatgcgccgccggacactggaaacaactataatgtagatgatattgctgagcagagtggactctaccgctaccacatgggcgggattgagggctggtgccagaagctctggacaacagaggccattgctttgctagatactgtagctgtgaagggccgtttccagctaacttcattaataaatggcgacaaccaaagtattgatatttcaaaaccaacaaggctggggaccaggactcaaagtgaagcagattatgatttggcaataaattctttaagattaatatcagcagcttataaaggcattggacataaattaaaagaaggtgagacctacttgtcacgtgacatgcagttcatgagtaaaacaatacaacatgaaggggtctactacccggcctccatcaagaaaatattaagagttggtccctggatcaacacaatattagatgatataaaaacttcaacagaaagtattggttctctaactcaagaactagaatataaaggtgaaagtttaatgagcagcctgctgctgaggaacttctggctctacagattatattcagtggatttaaaagatcattctttgtgtggaaagcagctctacagatccttaataaaagtgttaaaacatttgaagaggtgcttcaacctggagaaccttggggaatgtttggaattatttttaaatgtgcccatgcagtttggaggtgctgacccaaatgtcatctacaggagcttctacagaagaactccagattttctaacagaaagtataactcatctcatcctcattttaaaacattttagaagagatttggaattcaacaaagataatgtctccaaagctgttctttctttgctagagttcaccaagaatgattctgcagaatttgtaactttgatgagagatcctcaagcaattggtagtgagaggcaggccaagatcacttcagacatcaacagaacagctgtaacttcagtgttatcaaatgctccaaatgaaatatttagaacttcagctcttcattacagcagcacagaaaatgaattaaatggaatagcaagtggagtttctcctgtttatcctcatggtcttcgagttttatatgaaagtttacctttttataaagcagagaagattgtcaacatggtttctgggaccaagtccatcaccaacatactggagaagacatcagccatctcctacacagatataattcgagccaccaacatgatggtggagaacctcactttgctaacaagaataatgaaaccaggtgctgacacatctttggatcctgacacaatagtaataacaatattatcaaaaataataagagataaatcctgggatgttggtgatataattggtgtcacttccccatctcctgtctcctgcttcaaggtggtctacacatcaactctacaaaataattcagtagtaatagaaagatacacaacagacacctacacaagaggtaagagaggccccaccaagccctgggtgggcagcagcacacaggagaagaagtccatgcctgtctacaacagacaagttttaacaagaggacaaagagatcaaatagaaaatatagcaaagctggagtgggtgttttcttcagtagcaaatattgattctttgctaaatgagctcagcaccatgactttgggtctttctctaaggaaatgtagacaactttttccaacatatttaagtttaaacttcctgcacaggctttctgtcagcagcaggcccagagaatatccttcttctcttcctgcctacaggacaacaaattttcattttgatactggaccaataaataaagtgttaacagaaagatttggagatgaagatataaatttggtatttcaaaatgcaatatcatatggtctttccaccatgtctttggtggagcagtttactggtgtctgtccaaataaagttttgctggtgcccaagctacaagaaatacaactaatgaaagttccaatatttcaaggtggcttcaacctacaaagtataattccaataataaggcagcagcacatgttcctgcccaaccacatcactccagcccagtatattgaattatttctttcttcaaaacaatttcattcaagaataaatttaaaacacaacaacagatttaaacttgttttacaaaaagattattttaatggggagaacatgatagaaactttgtccacctgtttggcaggccactggatcatcattttgatgctaatgaaggagagtcaggggatatttgacaaggagtggtatgatggttttgtaacagaccacatgttcctggacctgcagctcttcctctcctccttcaagacatttctaactgtcttcaactttgcttatttaaaagttggttcaaatatagaagaaataacaggaaatcaagccaacctgctggagctgctggacctgggctactggaagaacatgtataaagtattttcagagaccaaggtgcggctggctttgctaaaacaagatttatcatttaattctgtgaagaacagcagcagcttccggcactggtttataaattctctacaagaagtacaatgtacttctgtgccttgggtggtaaatgtaacaagaaatccaactcatttaaaaggtgttctacagtacatgaagatgatagaaagtggcatgattcaaggttattcagcaaatatttcttcagttttaagtatcccatataattatccagacatggcgcacatgatgacaaaaataataagaaatcgaggccacatgtcctatgattatccaaagatgaagaaaagtttaactttctccatgacagacatgagtgacagctacatgctcaacctcttccccaaagtagaatgttcttacatgagtggttatttggataaactagatgatactctacaacttctaaagaaacctcctgttggaagaaaagttccttctgtggctttgccctggcaccactgcaacagatacaactttgtcttcagcagcacaggctgcaaagtttctgtcattgacatgcttccaaaacatttccgaagaagtaatttaaaagtaatatgttttattggagaaggggcgggcaacctcatgctaagagctgttttggaagttggtggaaatataaaattaatatatagatccttaaaagatcctgatgatcatcatgttcctgtagaatttctaagattaaaaccttgttatccttatattgatactggtggcagtttatctttggcttcaacagatgccaccaacaaggcccactgggattatttacatcttcactggacagatcctttaaatttaatagtatgtgatgcagaaataagtggtgtgaagcactggctaaaaattcttcacaggtggtatgagcacatgacttcctgcaagcactgtttaaaatcagaacatgataaatatttaataataaaatatcatgctcaagatgatttaatagatcttcctcatggtgtcaggctactaaaatgtaacatctgcctgggctccaaactaagtggcagtgagagctacctgctcattggtcttggtctttcaaataaacttcctgtctacagtgaagttcttcattcaaaacttcttttggcagaatgtcatcagtttcatcatccaaaatatttggatgtttctggaataaatacaaatataaaatccttaataccaatgttggattatccaataacttacaacaagatcaccactttgctagaaagtgtgcgggagctttcttcaaataaaaacaagaacaccatgtggattggaagaaatcctgtctaccataataaatggctaaagaggaaatatttcaacattttaaaatggctaaaatactgtatagaacttcctgccttcaggatggattataattcatttgagagaatagaaatgctttatccaaatttaagagatttggtagattctgtttctacttcagagctgaagaaagtaataaaagtaacaggcatcctcttcaggagcaacaccatgtgaattaatatgccaatgctaacatgtcatccccactactttttttgttcaatcactctcagtctagacatgtgagaagatcccaaacccaactgcacccctcccactcgaaatcagttgtagcctcagcagctccatctgccatgatctctgaccagcagcaaatgtattattattttaatttgtctatagttaacaaaaaattgatatctcacaggttgtaaacatagttcttttataattattgttagttaaactattgtgtttgacttcctttgggtatttttttcccgt

SEQ ID NO: 63 - skipped

SEQ ID NO: 64 - protein, 10 aa, Murine orthopneumovirus
TNFDRSDLET

SEQ ID NO: 65 - protein, 10 aa, Murine orthopneumovirus
SDSEESGDEA

SEQ ID NO: 66 - DNA, 14 bp, Murine orthopneumovirus
  source 1..14: mol_type = genomic DNA
aagattgctagtcc

SEQ ID NO: 67 - DNA, 17 bp, Murine orthopneumovirus
  source 1..17: mol_type = genomic DNA
caaagaccagaatcatg

SEQ ID NO: 68-69 - skipped (2 entries)

SEQ ID NO: 70 - DNA, 10 bp, Murine orthopneumovirus
  source 1..10: mol_type = genomic DNA
cacatcctag

SEQ ID NO: 71-73 - skipped (3 entries)